SEQUENCE LISTING (258 sequences)

SEQ ID NO: 1 - protein, 223 aa, Homo sapiens
KCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 223 aa, Homo sapiens
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 3 - protein, 223 aa, Homo sapiens
TCPRCPAPELLGGPSVFLFPPKPKDTLMISRTPEVTCVVVDVSHEDPEVQFKWYVDGVEVHNAKTKPREEQFNSTFRVVSVLTVLHQDWLNGKEYKCKVSNKALPAPIEKTISKTKGQPREPQVYTLPPSREEMTKNQVSLTCLVKGFYPSDIAVEWESSGQPENNYKTTPPMLDSDGSFFLYSKLTVDKSRWQQGNIFSCSVMHEALHNRFTQKSLSLSPGK

SEQ ID NO: 4 - protein, 225 aa, Homo sapiens
GPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 5 - protein, 214 aa, Mus sp.
MEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKE

SEQ ID NO: 6 - protein, 208 aa, Mus sp.
GPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRS

SEQ ID NO: 7 - protein, 223 aa, Homo sapiens
KSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWVSNGQPENNYKTTPPVLDSDGSFFLYSKLTVDKSRWQQGNVFSCSVIHEALHNHYTQKSLSLSPGK

SEQ ID NO: 8 - protein, 223 aa, Homo sapiens
KSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWVSNGQPENNYKTTPPVLDSDGSFFLYSKLTVDKSRWQQGNVFSCSVIHEALHNHYTQKSLSLSPGK

SEQ ID NO: 9 - protein, 227 aa, Homo sapiens
DKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWVSNGQPENNYKTTPPVLDSDGSFFLYSKLTVDKSRWQQGNVFSCSVIHEALHNHYTQKSLSLSPGK

SEQ ID NO: 10-30 - skipped (21 entries)

SEQ ID NO: 31 - protein, 306 aa, Methanococcus sp.
MDEFEMIKRNTSEIISEEELREVLKKDEKSAYIGFEPSGKIHLGHYLQIKKMIDLQNAGFDIIILLADLHAYLNQKGELDEIRKIGDYNKKVFEAMGLKAKYVYGSEFQLDKDYTLNVYRLALKTTLKRARRSMELIAREDENPKVAEVIYPIMQVNDIHYLGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMDLKNAVAEELIKILEPIRKRL

SEQ ID NO: 32 - protein, 454 aa, Methanosarcina mazei
MDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLAPNLYNYLRKLDRALPDPIKIFEIGPCYRKESDGKEHLEEFTMLNFCQMGSGCTRENLESIITDFLNHLGIDFKIVGDSCMVYGDTLDVMHGDLELSSAVVGPIPLDREWGIDKPWIGAGFGLERLLKVKHDFKNIKRAARSGSYYNGISTNL

SEQ ID NO: 33 - protein, 419 aa, Methanosarcina barkeri
MDKKPLDVLISATGLGMSRTGTLHKIKHHEVSRSKIYIEMACGDHLVVNNSRSCRTARAFRHHKYRKTCKRCRVSDEDINNFLTRSTESKNSVKVRVVSAPKVKKAMPKSVSRAPKPLENSVSAKASTNTSRSVPSPAKSTPNSSVPASAPAPSLTRSQLDRVEALLSPEDKISLNMAKPFRELEPELVTRRKNDFQRLYTNDREDYLGKLERDITKFFVDRGFLEIKSPILIPAEYVERMGINNDTELSKQIFRVDKNLCLRPMLAPTLYNYLRKLDRILPGPIKVFEVGPCYRKESDGKEHLEEFTMVNFCQMGSGCTRENLEALIKEFLDYLEIDFEIVGDSCMVYGDTLDIMHGDLELSSAVVGPVSLDREWGIDKPWIGAGFGLERLLKVMHGFKNIKRASRSESYYNGISTNL

SEQ ID NO: 34 - protein, 424 aa, Escherichia coli
MASSNLIKQLQERGLVAQVTDEEALAERLAQGPIALYCGFDPTADSLHLGHLVPLLCLKRFQQAGHKPVALVGGATGLIGDPSFKAAERKLNTEETVQEWVDKIRKQVAPFLDFDCGENSAIAANNYDWFGNMNVLTFLRDIGKHFSVNQMINKEAVKQRLNREDQGISFTEFSYNLLQGYDFACLNKQYGVVLQIGGSDQWGNITSGIDLTRRLHQNQVFGLTVPLITKADGTKFGKTEGGAVWLDPKKTSPYKFYQFWINTADADVYRFLKFFTFMSIEEINALEEEDKNSGKAPRAQYVLAEQVTRLVHGEEGLQAAKRITECLFSGSLSALSEADFEQLAQDGVPMVEMEKGADLMQALVDSELQPSRGQARKTIASNAITINGEKQSDPEYFFKEEDRLFGRFTLLRRGKKNYCLICWK

SEQ ID NO: 35-40 - skipped (6 entries)

SEQ ID NO: 41 - protein, 306 aa, synthetic construct
  source 1..306: note = Methanococcus jannaschii tyrosyl-tRNA synthetase variant
MDEFEMIKRNTSEIISEEELREVLKKDEKSALIGFEPSGKIHLGHYLQIKKMIDLQNAGFDIIILLADLHAYLNQKGELDEIRKIGDYNKKVFEAMGLKAKYVYGSEFQLDKDYTLNVYRLALKTTLKRARRSMELIAREDENPKVAEVIYPIMQVNGCHYRGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMRLKNAVAEELIKILEPIRKRL

SEQ ID NO: 42 - protein, 306 aa, synthetic construct
  source 1..306: note = Methanococcus jannaschii tyrosyl-tRNA synthetase variant
MDEFEMIKRNTSEIISEEELREVLKKDEKSAQIGFEPSGKIHLGHYLQIKKMIDLQNAGFDIIIELADLHAYLNQKGELDEIRKIGDYNKKVFEAMGLKAKYVYGSEGLLDKDYTLNVYRLALKTTLKRARRSMELIAREDENPKVAEVIYPIMQVNSIHYYGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMRLKNAVAEELIKILEPIRKRL

SEQ ID NO: 43 - protein, 306 aa, synthetic construct
  source 1..306: note = Methanococcus jannaschii tyrosyl-tRNA synthetase variant
MDEFEMIKRNTSEIISEEELREVLKKDEKSALIGFEPSGKIHLGHYLQIKKMIDLQNAGFDIIILLADLHAYLNQKGELDEIRKIGDYNKKVFEAMGLKAKYVYGSEFQLDKDYTLNVYRLALKTTLKRARRSMELIAREDENPKVAEVIYPIMQVNPAHYQGVDVVVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMRLKNAVAEELIKILEPIRKRL

SEQ ID NO: 44 - protein, 306 aa, synthetic construct
  source 1..306: note = Methanococcus jannaschii tyrosyl-tRNA synthetase variant
MDEFEMIKRNTSEIISEEELREVLKKDEKSAAIGFEPSGKIHLGHYLQIKKMIDLQNAGFDIIILLADLHAYLNQKGELDEIRKIGDYNKKVFEAMGLKAKYVYGSPFQLDKDYTLNVYRLALKTTLKRARRSMELIAREDENPKVAEVIYPIMQVNAIHYAGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMRLKNAVAEELIKILEPIRKRL

SEQ ID NO: 45 - protein, 306 aa, synthetic construct
  source 1..306: note = Methanococcus jannaschii tyrosyl-tRNA synthetase variant
MDEFEMIKRNTSEIISEEELREVLKKDEKSAGIGFEPSGKIHLGHYLQIKKMIDLQNAGFDIIILLADLHAYLNQKGELDEIRKIGDYNKKVFEAMGLKAKYVYGSPFQLDKDYTLNVYRLALKTTLKRARRSMELIAREDENPKVAEVIYPIMQVNAIHYAGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMRLKNAVAEELIKILEPIRKRL

SEQ ID NO: 46 - protein, 306 aa, synthetic construct
MDEFEMIKRNTSEIISEEELREVLKKDEKSATIGFEPSGKIHLGHYLQIKKMIDLQNAGFDIIILLADLHAYLNQKGELDEIRKIGDYNKKVFEAMGLKAKYVYGSNFQLDKDYTLNVYRLALKTTLKRARRSMELIAREDENPKVAEVIYPIMQVNPLHYQGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMRLKNAVAEELIKILEPIRKRL

SEQ ID NO: 47 - protein, 306 aa, synthetic construct
  source 1..306: note = Methanococcus jannaschii tyrosyl-tRNA synthetase variant
MDEFEMIKRNTSEIISEEELREVLKKDEKSATIGFEPSGKIHLGHYLQIKKMIDLQNAGFDIIILLADLHAYLNQKGELDEIRKIGDYNKKVFEAMGLKAKYVYGSTFQLDKDYTLNVYRLALKTTLKRARRSMELIAREDENPKVAEVIYPIMQVNPLHYAGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMRLKNAVAEELIKILEPIRKRL

SEQ ID NO: 48 - protein, 306 aa, synthetic construct
  source 1..306: note = Methanococcus jannaschii tyrosyl-tRNA synthetase variant
MDEFEMIKRNTSEIISEEELREVLKKDEKSAGIGFEPSGKIHLGHYLQIKKMIDLQNAGFDIIILLADLHAYLNQKGELDEIRKIGDYNKKVFEAMGLKAKYVYGSPFQLDKDYTLNVYRLALKTTLKRARRSMELIAREDENPKVAEVIYPIMQVNTSHYLGADVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMRLKNAVAEELIKILEPIRKRL

SEQ ID NO: 49 - protein, 306 aa, synthetic construct
  source 1..306: note = Methanococcus jannaschii tyrosyl-tRNA synthetase variant
MDEFEMIKRNTSEIISEEELREVLKKDEKSALIGFEPSGKIHLGHYLQIKKMIDLQNAGFDIIIVLADLHAYLNQKGELDEIRKIGDYNKKVFEAMGLKAKYVYGSEWMLDKDYTLNVYRLALKTTLKRARRSMELIAREDENPKVAEVIYPIMQVNGAHYLGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMDLKNAVAEELIKILEPIRKRL

SEQ ID NO: 50 - protein, 306 aa, synthetic construct
MDEFEMIKRNTSEIISEEELREVLKKDEKSAEIGFEPSGKIHLGHYLQIKKMIDLQNAGFDIIIHLGDLGAYLNQKGELDEIRKIGDYNKKVFEAMGLKAKYVYGSEYHLDKDYTLNVYRLALKTTLKRARRSMELIAREDENPKVAEVIYPIMQVNGIHYGGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMDLKNAVAEELIKILEPIRKRL

SEQ ID NO: 51 - protein, 306 aa, synthetic construct
MDEFEMIKRNTSEIISEEELREVLKKDEKSALIGFEPSGKIHLGHYLQIKKMIDLQNAGFDIIILLADLHAYLNQKGELDEIRKIGDYNKKVFEAMGLKAKYVYGSEFQLDKDYTLNVYRLALKTTLKRARRSMELIAREDENPKVAEVIYPIMQVNPAHYQGVDVVVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMDLKNAVAEELIKILEPIRKRL

SEQ ID NO: 52 - protein, 306 aa, synthetic construct
  source 1..306: note = Methanococcus jannaschii tyrosyl-tRNA synthetase variant
MDEFEMIKRNTSEIISEEELREVLKKDEKSAHIGFEPSGKIHLGHYLQIKKMIDLQNAGFDIIIHLADLHAYLNQKGELDEIRKIGDYNKKVFEAMGLKAKYVYGSEWMLDKDYTLNVYRLALKTTLKRARRSMELIAREDENPKVAEVIYPIMQVNGIHYKGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMDLKNAVAEELIKILEPIRKRL

SEQ ID NO: 53 - protein, 306 aa, synthetic construct
  source 1..306: note = Methanococcus jannaschii tyrosyl-tRNA synthetase variant
MDEFEMIKRNTSEIISEEELREVLKKDEKSAGIGFEPSGKIHLGHYLQIKKMIDLQNAGFDIIIVLADLHAYLNQKGELDEIRKIGDYNKKVFEAMGLKAKYVYGSEADLDKDYTLNVYRLALKTTLKRARRSMELIAREDENPKVAEVIYPIMQVNSIHYRGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMDLKNAVAEELIKILEPIRKRL

SEQ ID NO: 54 - protein, 306 aa, synthetic construct
  source 1..306: note = Methanococcus jannaschii tyrosyl-tRNA synthetase variant
MDEFEMIKRNTSEIISEEELREVLKKDEKSAGIGFEPSGKIHLGHYLQIKKMIDLQNAGFDIIIYLADLAAYLNQKGELDEIRKIGDYNKKVFEAMGLKAKYVYGSEFQLDKDYTLNVYRLALKTTLKRARRSMELIAREDENPKVAEVIYPIMEVNGWHYSGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMDLKNAVAEELIKILEPIRKRL

SEQ ID NO: 55 - protein, 306 aa, synthetic construct
  source 1..306: note = Methanococcus jannaschii tyrosyl-tRNA synthetase variant
MDEFEMIKRNTSEIISEEELREVLKKDEKSAGIGFEPSGKIHLGHYLQIKKMIDLQNAGFDIIIGLADLHAYLNQKGELDEIRKIGDYNKKVFEAMGLKAKYVYGSEEQLDKDYTLNVYRLALKTTLKRARRSMELIAREDENPKVAEVIYPIMQVNSIHYEGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMDLKNAVAEELIKILEPIRKRL

SEQ ID NO: 56 - protein, 306 aa, synthetic construct
  source 1..306: note = Methanococcus jannaschii tyrosyl-tRNA synthetase variant
MDEFEMIKRNTSEIISEEELREVLKKDEKSAGIGFEPSGKIHLGHYLQIKKMIDLQNAGFDIIILLADLHAYLNQKGELDEIRKIGDYNKKVFEAMGLKAKYVYGSPFQLDKDYTLNVYRLALKTTLKRARRSMELIAREDENPKVAEVIYPIMQVNTSHYLGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMDLKNAVAEELIKILEPIRKRL

SEQ ID NO: 57 - protein, 306 aa, synthetic construct
  source 1..306: note = Methanococcus jannaschii tyrosyl-tRNA synthetase variant
MDEFEMIKRNTSEIISEEELREVLKKDEKSAHIGFEPSGKIHLGHYLQIKKMIDLQNAGFDIIILLADLCAYLNQKGELDEIRKIGDYNKKVFEAMGLKAKYVYGSEFQLDKDYTLNVYRLALKTTLKRARRSMELIAREDENPKVAEVIYPIMQVNSAHYRGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMDLKNAVAEELIKILEPIRKRL

SEQ ID NO: 58 - protein, 306 aa, synthetic construct
  source 1..306: note = Methanococcus jannaschii tyrosyl-tRNA synthetase variant
MDEFEMIKRNTSEIISEEELREVLKKDEKSAGIGFEPSGKIHLGHYLQIKKMIDLQNAGFDIIIELADLHAYLNQKGELDEIRKIGDYNKKVFEAMGLKAKYVYGSEWMLDKDYTLNVYRLALKTTLKRARRSMELIAREDENPKVAEVIYPIMQVNSIHYKGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMDLKNAVAEELIKILEPIRKRL

SEQ ID NO: 59 - protein, 306 aa, synthetic construct
  source 1..306: note = Methanococcus jannaschii tyrosyl-tRNA synthetase variant
MDEFEMIKRNTSEIISEEELREVLKKDEKSALIGFEPSGKIHLGHYLQIKKMIDLQNAGFDIIIALADLHAYLNQKGELDEIRKIGDYNKKVFEAMGLKAKYVYGSESHLDKDYTLNVYRLALKTTLKRARRSMELIAREDENPKVAEVIYPIMQVNAIHYMGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMDLKNAVAEELIKILEPIRKRL

SEQ ID NO: 60 - protein, 306 aa, synthetic construct
  source 1..306: note = Methanococcus jannaschii tyrosyl-tRNA synthetase variant
MDEFEMIKRNTSEIISEEELREVLKKDEKSAVIGFEPSGKIHLGHYLQIKKMIDLQNAGFDIIIALADLHAYLNQKGELDEIRKIGDYNKKVFEAMGLKAKYVYGSEQWLDKDYTLNVYRLALKTTLKRARRSMELIAREDENPKVAEVIYPIMQVNAIHYKGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMDLKNAVAEELIKILEPIRKRL

SEQ ID NO: 61 - protein, 306 aa, synthetic construct
MDEFEMIKRNTSEIISEEELREVLKKDEKSAGIGFEPSGKIHLGHYLQIKKMIDLQNAGFDIIIYLADLHAYLNQKGELDEIRKIGDYNKKVFEAMGLKAKYVYGSEHGLDKDYTLNVYRLALKTTLKRARRSMELIAREDENPKVAEVIYPIMQVNGLHYQGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMRLKNAVAEELIKILEPIRKRL

SEQ ID NO: 62 - protein, 306 aa, synthetic construct
  source 1..306: note = Methanococcus jannaschii tyrosyl-tRNA synthetase variant
MDEFEMIKRNTSEIISEEELREVLKKDEKSAVIGFEPSGKIHLGHYLQIKKMIDLQNAGFDIIIYLADLHAYLNQKGELDEIRKIGDYNKKVFEAMGLKAKYVYGSEHGLDKDYTLNVYRLALKTTLKRARRSMELIAREDENPKVAEVIYPIMQVNGIHYEGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMRLKNAVAEELIKILEPIRKRL

SEQ ID NO: 63 - protein, 306 aa, synthetic construct
  source 1..306: note = Methanococcus jannaschii tyrosyl-tRNA synthetase variant
MDEFEMIKRNTSEIISEEELREVLKKDEKSAGIGFEPSGKIHLGHYLQIKKMIDLQNAGFDIIIYLADLHAYLNQKGELDEIRKIGDYNKKVFEAMGLKAKYVYGSEHGLDKDYTLNVYRLALKTTLKRARRSMELIAREDENPKVAEVIYPIMQVNGIHYEGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMRLKNAVAEELIKILEPIRKRL

SEQ ID NO: 64 - protein, 306 aa, synthetic construct
  source 1..306: note = Methanococcus jannaschii tyrosyl-tRNA synthetase variant
MDEFEMIKRNTSEIISEEELREVLKKDEKSAAIGFEPSGKIHLGHYLQIKKMIDLQNAGFDIIIYLADLHAYLNQKGELDEIRKIGDYNKKVFEAMGLKAKYVYGSEHGLDKDYTLNVYRLALKTTLKRARRSMELIAREDENPKVAEVIYPIMQVNGIHYEGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMRLKNAVAEELIKILEPIRKRL

SEQ ID NO: 65 - protein, 454 aa, synthetic construct
  source 1..454: note = Methanococcus jannaschii tyrosyl-tRNA synthetase variant
MDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMMAPNLLNYARKLDRALPDPIKIFEIGPCYRKESDGKEHLEEFTMLNFFQMGSGCTRENLESIITDFLNHLGIDFKIVGDSCMVYGDTLDVMHGDLELSSAVVGPIPLDREWGIDKPWIGAGFGLERLLKVKHDFKNIKRAARSGSYYNGISTNL

SEQ ID NO: 66 - protein, 454 aa, synthetic construct
  source 1..454: note = Methanococcus jannaschii tyrosyl-tRNA synthetase variant
MDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMMAPNLLNYARKLDRALPDPIKIFEIGPCYRKESDGKEHLEEFTMLNFFQMGSGCTRENLESIITDFLNHLGIDFKIVGDSCMVWGDTLDVMHGDLELSSAVVGPIPLDREWGIDKPWIGAGFGLERLLKVKHDFKNIKRAARSGSYYNGISTNL

SEQ ID NO: 67 - protein, 454 aa, synthetic construct
MDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLAPNLANYLRKLDRALPDPIKIFEIGPCYRKESDGKEHLEEFTMLNFCQMGSGCTRENLESIITDFLNHLGIDFKIVGDSCMVFGDTLDVMHGDLELSSAVVGPIPLDREWGIDKPWIGAGFGLERLLKVKHDFKNIKRAARSGSYYNGISTNL

SEQ ID NO: 68 - protein, 454 aa, synthetic construct
  source 1..454: note = Methanococcus jannaschii tyrosyl-tRNA synthetase variant
MDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLAPNLYNYLRKLDRALPDPIKIFEIGPCYRKESDGKEHLEEFTMLNFAQMGSGCTRENLESIITDFLNHLGIDFKIVGDSCMVYGDTLDVMHGDLELSSAVVGPIPLDREWGIDKPWIGAGFGLERLLKVKHDFKNIKRAARSGSYYNGISTNL

SEQ ID NO: 69 - protein, 454 aa, synthetic construct
MDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLTPNLYNYLRKLDRALPDPIKIFEIGPCYRKESDGKEHLEEFTMLNFAQMGSGCTRENLESIITDFLNHLGIDFKIVGDSCMVFGDTLDVMHGDLELSSAVVGPIPLDREWGIDKPTIGAGFGLERLLKVKHDFKNIKRAARSGSYYNGISTNL

SEQ ID NO: 70 - protein, 454 aa, synthetic construct
  source 1..454: note = Methanococcus jannaschii tyrosyl-tRNA synthetase variant
MDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLAPNLLNYLRKLDRALPDPIKIFEIGPCYRKESDGKEHLEEFTMLNFIQMGSGCTRENLESIITDFLNHLGIDFKIVGDSCMVFGDTLDVMHGDLELSSAVVGPIPLDREWGIDKPWIGAGFGLERLLKVKHDFKNIKRAARSGSYYNGISTNL

SEQ ID NO: 71 - protein, 454 aa, synthetic construct
MDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLAPNLYNYLRKLDRALPDPIKIFEIGPCYRKESDGKEHLEEFTMLAFAQMGSGCTRENLESIITDFLNHLGIDFKIVGDSCMVYGDTLDVMHGDLELSSAVVGPIPLDREWGIDKPWIGAGFGLERLLKVKHDFKNIKRAARSGSYYNGISTNL

SEQ ID NO: 72 - protein, 454 aa, synthetic construct; identical to SEQ ID NO: 67

SEQ ID NO: 73 - protein, 419 aa, synthetic construct
  source 1..419: note = Methanococcus jannaschii tyrosyl-tRNA synthetase variant
MDKKPLDVLISATGLGMSRTGTLHKIKHHEVSRSKIYIEMACGDHLVVNNSRSCRTARAFRHHKYRKTCKRCRVSDEDINNFLTRSTESKNSVKVRVVSAPKVKKAMPKSVSRAPKPLENSVSAKASTNTSRSVPSPAKSTPNSSVPASAPAPSLTRSQLDRVEALLSPEDKISLNMAKPFRELEPELVTRRKNDFQRLYTNDREDYLGKLERDITKFFVDRGFLEIKSPILIPAEYVERMGINNDTELSKQIFRVDKNLCLRPMLAPTLYNYARKLDRILPGPIKVFEVGPCYRKESDGKEHLEEFTMVNFAQMGSGCTRENLEALIKEFLDYLEIDFEIVGDSCMVFGDTLDIMHGDLELSSAVVGPVSLDREWGIDKPWIGAGFGLERLLKVMHGFKNIKRASRSESYYNGISTNL

SEQ ID NO: 74 - protein, 419 aa, synthetic construct
  source 1..419: note = Methanococcus jannaschii tyrosyl-tRNA synthetase variant
MDKKPLDVLISATGLGMSRTGTLHKIKHHEVSRSKIYIEMACGDHLVVNNSRSCRTARAFRHHKYRKTCKRCRVSDEDINNFLTRSTESKNSVKVRVVSAPKVKKAMPKSVSRAPKPLENSVSAKASTNTSRSVPSPAKSTPNSSVPASAPAPSLTRSQLDRVEALLSPEDKISLNMAKPFRELEPELVTRRKNDFQRLYTNDREDYLGKLERDITKFFVDRGFLEIKSPILIPAEYVERMGINNDTELSKQIFRVDKNLCLRPMLAPTLMNYGRKLDRILPGPIKVFEVGPCYRKESDGKEHLEEFTMVNFAQMGSGCTRENLEALIKEFLDYLEIDFEIVGDSCMVYGDTLDIMHGDLELSSAVVGPVSLDREWGIDKPWIGAGFGLERLLKVMHGFKNIKRASRSESYYNGISTNL

SEQ ID NO: 75 - protein, 419 aa, synthetic construct
  source 1..419: note = Methanococcus jannaschii tyrosyl-tRNA synthetase variant
MDKKPLDVLISATGLGMSRTGTLHKIKHHEVSRSKIYIEMACGDHLVVNNSRSCRTARAFRHHKYRKTCKRCRVSDEDINNFLTRSTESKNSVKVRVVSAPKVKKAMPKSVSRAPKPLENSVSAKASTNTSRSVPSPAKSTPNSSVPASAPAPSLTRSQLDRVEALLSPEDKISLNMAKPFRELEPELVTRRKNDFQRLYTNDREDYLGKLERDITKFFVDRGFLEIKSPILIPAEYVERMGINNDTELSKQIFRVDKNLCLRPMLAPTLMNYGRKLDRILPGPIKVFEVGPCYRKESDGKEHLEEFTMVNFAQMGSGCTRENLEALIKEFLDYLEIDFEIVGDSCMVWGDTLDIMHGDLELSSAVVGPVSLDREWGIDKPWIGAGFGLERLLKVMHGFKNIKRASRSESYYNGISTNL

SEQ ID NO: 76 - protein, 419 aa, synthetic construct
  source 1..419: note = Methanococcus jannaschii tyrosyl-tRNA synthetase variant
MDKKPLDVLISATGLGMSRTGTLHKIKHHEVSRSKIYIEMACGDHLVVNNSRSCRTARAFRHHKYRKTCKRCRVSDEDINNFLTRSTESKNSVKVRVVSAPKVKKAMPKSVSRAPKPLENSVSAKASTNTSRSVPSPAKSTPNSSVPASAPAPSLTRSQLDRVEALLSPEDKISLNMAKPFRELEPELVTRRKNDFQRLYTNDREDYLGKLERDITKFFVDRGFLEIKSPILIPAEYVERMGINNDTELSKQIFRVDKNLCLRPMLAPTIFNYGRKLDRILPGPIKVFEVGPCYRKESDGKEHLEEFTMVNFFQMGSGCTRENLEALIKEFLDYLEIDFEIVGDSCMVFGDTLDIMHGDLELSSAVVGPVSLDREWGIDKPWIGAGFGLERLLKVMHGFKNIKRASRSESYYNGISTNL

SEQ ID NO: 77 - protein, 419 aa, synthetic construct
MDLLAELQWRGLVNQTTDEDGLRKLLNEERVTLVCGFDPTADSLHIGNLAAILTLRRFQQAGHRPIALVGGATGLIGDPSGKKSERTLNAKETVEAWSARIKEQLGRFLDFEADGNPAKIKNNYDWIGPLDVITFLRDVGKHFSVNYMMAKESVQSRIETGISFTEFSYMMLQAYSMLRAYETEGCRLQIGGSDQWGNITAGLELIRKTKGEARAFGLTIPLVTKADGTKFGKTESGTIWLDKEKTSPYEFYQFWINTDRRDVIRYLKYFTFLSKEEIEALEQELREAPEKRAAQKALAEEVTKLVHGEEALRQAIRISEALFSGDIANLTAAEIEQGFKDVPSFVHEGGDVPLVELLVSAGISPSKRQAREDIQNGAIYVNGERLQDVGAILTAEHRLEGRFTVIRRGKKKYYLIRYA

SEQ ID NO: 78 - protein, 424 aa, synthetic construct
  source 1..424: note = E. coli tyrosyl-tRNA synthetase variant
MASSNLIKQLQERGLVAQVTDEEALAERLAQGPIALVCGFDPTADSLHLGHLVPLLCLKRFQQAGHKPVALVGGATGLIGDPSFKAAERKLNTEETVQEWVDKIRKQVAPFLDFDCGENSAIAANNYDWFGNMNVLTFLRDIGKHFSVNQMINKEAVKQRLNREDQGISFTEFSYNLLQGYSMACLNKQYGVVLQIGGSDQWGNITSGIDLTRRLHQNQVFGLTVPLITKADGTKFGKTEGGAVWLDPKKTSPYKFYQFWINTARADVYRFLKFFTFMSIEEINALEEEDKNSGKAPRAQYVLAEQVTRLVHGEEGLQAAKRITECLFSGSLSALSEADFEQLAQDGVPMVEMEKGADLMQALVDSELQPSRGQARKTIASNAITINGEKQSDPEYFFKEEDRLFGRFTLLRRGKKNYCLICWK

SEQ ID NO: 79 - protein, 424 aa, synthetic construct
MASSNLIKQLQERGLVAQVTDEEALAERLAQGPIALVCGFDPTADSLHLGHLVPLLCLKRFQQAGHKPVALVGGATGLIGDPSFKAAERKLNTEETVQEWVDKIRKQVAPFLDFDCGENSAIAANNYDWFGNMNVLTFLRDIGKHFSVNQMINKEAVKQRLNREDQGISFTEFSYNLLQGYSMACANKQYGVVLQIGGSDQWGNITSGIDLTRRLHQNQVFGLTVPLITKADGTKFGKTEGGAVWLDPKKTSPYKFYQFWINTARADVYRFLKFFTFMSIEEINALEEEDKNSGKAPRAQYVLAEQVTRLVHGEEGLQAAKRITECLFSGSLSALSEADFEQLAQDGVPMVEMEKGADLMQALVDSELQPSRGQARKTIASNAITINGEKQSDPEYFFKEEDRLFGRFTLLRRGKKNYCLICWK

SEQ ID NO: 80 - protein, 424 aa, synthetic construct
  source 1..424: note = E. coli tyrosyl-tRNA synthetase variant
MASSNLIKQLQERGLVAQVTDEEALAERLAQGPIALSCGFDPTADSLHLGHLVPLLCLKRFQQAGHKPVALVGGATGLIGDPSFKAAERKLNTEETVQEWVDKIRKQVAPFLDFDCGENSAIAANNYDWFGNMNVLTFLRDIGKHFSVNQMINKEAVKQRLNREDQGISFTEFSYNLLQGYTMACVNKQYGVVLQIGGSDQWGNITSGIDLTRRLHQNQVFGLTVPLITKADGTKFGKTEGGAVWLDPKKTSPYKFYQFWINTARADVYRFLKFFTFMSIEEINALEEEDKNSGKAPRAQYVLAEQVTRLVHGEEGLQAAKRITECLFSGSLSALSEADFEQLAQDGVPMVEMEKGADLMQALVDSELQPSRGQARKTIASNAITINGEKQSDPEYFFKEEDRLFGRFTLLRRGKKNYCLICWK

SEQ ID NO: 81 - protein, 424 aa, synthetic construct
  source 1..424: note = E. coli tyrosyl-tRNA synthetase variant
MASSNLIKQLQERGLVAQVTDEEALAERLAQGPIALICGFDPTADSLHLGHLVPLLCLKRFQQAGHKPVALVGGATGLIGDPSFKAAERKLNTEETVQEWVDKIRKQVAPFLDFDCGENSAIAANNYDWFGNMNVLTFLRDIGKHFSVNQMINKEAVKQRLNREDQGISFTEFSYNLLQGYGMACANKQYGVVLQIGGSDQWGNITSGIDLTRRLHQNQVFGLTVPLITKADGTKFGKTEGGAVWLDPKKTSPYKFYQFWINTARADVYRFLKFFTFMSIEEINALEEEDKNSGKAPRAQYVLAEQVTRLVHGEEGLQAAKRITECLFSGSLSALSEADFEQLAQDGVPMVEMEKGADLMQALVDSELQPSRGQARKTIASNAITINGEKQSDPEYFFKEEDRLFGRFTLLRRGKKNYCLICWK

SEQ ID NO: 82-100 - skipped (19 entries)

SEQ ID NO: 101 - RNA, 77 nt, synthetic construct
  source 1..77: mol_type = other RNA
ccggcggtagttcagcagggcagaacggcggactctaaatccgcatggcaggggttcaaatcccctccgccggacca

SEQ ID NO: 102 - RNA, 77 nt, synthetic construct
  source 1..77: mol_type = other RNA; note = Nucleotide encoding Methanococcus jannaschii tRNA
ccggcggtagttcagcagggcagaacggcggactttaaatccgcatggcaggggttcaaatcccctccgccggacca

SEQ ID NO: 103 - RNA, 77 nt, synthetic construct
  source 1..77: mol_type = other RNA; note = Nucleotide encoding Methanococcus jannaschii tRNA
ccggcggtagttcagcagggcagaacggcggacttcaaatccgcatggcaggggttcaaatcccctccgccggacca

SEQ ID NO: 104 - RNA, 78 nt, synthetic construct
  source 1..78: mol_type = other RNA; note = Nucleotide encoding Methanococcus jannaschii tRNA
ccggcggtagttcagcagggcagaacggcggacttctaaatccgcatggcaggggttcaaatcccctccgccggacca

SEQ ID NO: 105 - RNA, 72 nt, synthetic construct
  source 1..72: mol_type = other RNA; note = Nucleotide encoding Methanosarcina species tRNAPyl
ggaaacctgatcatgtagatcgaatggactctaaatccgttcagccgggttagattcccggggtttccgcca

SEQ ID NO: 106 - RNA, 72 nt, synthetic construct
  source 1..72: mol_type = other RNA; note = Nucloetide Methanosarcina species tRNAPyl
ggaaacctgatcatgtagatcgaatggactttaaatccgttcagccgggttagattcccggggtttccgcca

SEQ ID NO: 107 - RNA, 72 nt, synthetic construct
  source 1..72: mol_type = other RNA; note = Nucleotide encoding Methanosarcina species tRNAPyl
ggaaacctgatcatgtagatcgaatggacttcaaatccgttcagccgggttagattcccggggtttccgcca

SEQ ID NO: 108 - RNA, 73 nt, synthetic construct
  source 1..73: mol_type = other RNA; note = Nucleotide encoding Methanosarcina species tRNAPyl
ggaaacctgatcatgtagatcgaatggacttctaaatccgttcagccgggttagattcccggggtttccgcca

SEQ ID NO: 109 - RNA, 72 nt, synthetic construct
  source 1..72: mol_type = other RNA
ggggggtggatcgaatagatcacacggactctaaattcgtgcaggcgggtgaaactcccgtactccccgcca

SEQ ID NO: 110 - RNA, 72 nt, synthetic construct
  source 1..72: mol_type = other RNA; note = Nucleotide encoding Desulfitobacterium hafniense tRNAPyl
ggggggtggatcgaatagatcacacggactttaaattcgtgcaggcgggtgaaactcccgtactccccgcca

SEQ ID NO: 111 - RNA, 72 nt, synthetic construct
  source 1..72: mol_type = other RNA; note = Nucleotide encoding Desulfitobacterium hafniense tRNAPyl
ggggggtggatcgaatagatcacacggacttcaaattcgtgcaggcgggtgaaactcccgtactccccgcca

SEQ ID NO: 112 - RNA, 73 nt, synthetic construct
  source 1..73: mol_type = other RNA; note = Nucleotide encoding Desulfitobacterium hafniense tRNAPyl
ggggggtggatcgaatagatcacacggacttctaaattcgtgcaggcgggtgaaactcccgtactccccgcca

SEQ ID NO: 113 - RNA, 85 nt, synthetic construct
  source 1..85: mol_type = other RNA; note = Nucleotide encoding Escherichia coli tRNATyr
ggtggggttcccgagcggccaaagggagcagactctaaatctgccgtcacagacttcgaaggttcgaatccttcccccaccacca

SEQ ID NO: 114 - RNA, 85 nt, synthetic construct
  source 1..85: mol_type = other RNA; note = Nucleotide encoding Escherichia coli tRNATyr
ggtggggttcccgagcggccaaagggagcagactttaaatctgccgtcacagacttcgaaggttcgaatccttcccccaccacca

SEQ ID NO: 115 - RNA, 85 nt, synthetic construct
  source 1..85: mol_type = other RNA; note = Nucleotide encoding Escherichia coli tRNATyr
ggtggggttcccgagcggccaaagggagcagacttcaaatctgccgtcacagacttcgaaggttcgaatccttcccccaccacca

SEQ ID NO: 116 - RNA, 86 nt, synthetic construct
  source 1..86: mol_type = other RNA; note = Nucleotide encoding Escherichia coli tRNATyr
ggtggggttcccgagcggccaaagggagcagacttctaaatctgccgtcacagacttcgaaggttcgaatccttcccccaccacca

SEQ ID NO: 117 - RNA, 85 nt, synthetic construct
  source 1..85: mol_type = other RNA; note = Nucleotide encoding Bacillus stearothermophilus tRNATyr
ggaggggtagcgaagtggctaaacgcggcggactctaaatccgctccctcagggttcggcagttcgaatctgcccccctccacca

SEQ ID NO: 118 - RNA, 85 nt, synthetic construct
  source 1..85: mol_type = other RNA; note = Nucleotide encoding Bacillus stearothermophilus tRNATyr
ggaggggtagcgaagtggctaaacgcggcggactttaaatccgctccctcagggttcggcagttcgaatctgcccccctccacca

SEQ ID NO: 119 - RNA, 85 nt, synthetic construct
  source 1..85: mol_type = other RNA; note = Nucleotide encoding Bacillus stearothermophilus tRNATyr
ggaggggtagcgaagtggctaaacgcggcggacttcaaatccgctccctcagggttcggcagttcgaatctgcccccctccacca

SEQ ID NO: 120 - RNA, 86 nt, synthetic construct
  source 1..86: mol_type = other RNA
ggaggggtagcgaagtggctaaacgcggcggacttctaaatccgctccctcagggttcggcagttcgaatctgcccccctccacca

SEQ ID NO: 121 - protein, 5 aa, synthetic construct
RRRRR

SEQ ID NO: 122 - protein, 6 aa, synthetic construct
HHHHHH

SEQ ID NO: 123 - protein, 19 aa, synthetic construct
SGLNDIFEAQKIEWHELEL

SEQ ID NO: 124 - protein, 8 aa, synthetic construct
DYKDDDDK

SEQ ID NO: 125 - protein, 8 aa, synthetic construct
WSHPQFEK

SEQ ID NO: 126 - protein, 10 aa, synthetic construct
EQKLISEEDL

SEQ ID NO: 127 - protein, 15 aa, synthetic construct
KETAAAKFERQHMDS

SEQ ID NO: 128 - protein, 26 aa, synthetic construct
KRRWKKNFIAVSAANRFKKISSSGAL

SEQ ID NO: 129 - protein, 38 aa, synthetic construct
MDEKTTGWRGGHVVEGLAGELEQLRARLEHHPQGQREP

SEQ ID NO: 130 - protein, 71 aa, synthetic construct
MKIEEGKLTNPGVSAWQVNTAYTAGQLVTYNGKTYKCLQPHTSLAGWEPSNVPALWQLQNNGNNGLELRHG

SEQ ID NO: 131 - protein, 220 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSD

SEQ ID NO: 132 - protein, 368 aa, synthetic construct
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTN

SEQ ID NO: 133 - protein, 128 aa, synthetic construct
MAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 134 - protein, 239 aa, synthetic construct
MVSKGEELFTGVVPILVELDGDVNGHKFSVSGEGEGDATYGKLTLKFICTTGKLPVPWPTLVTTFSYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYK

SEQ ID NO: 135 - protein, 550 aa, synthetic construct
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKL

SEQ ID NO: 136 - protein, 524 aa, synthetic construct
MISPFLLLAIGTCFASSLVPEKEKDPKYWRDQAQQTLKNALRLQTLNTNVAKNVIMFLGDGMGVSTVTAARILKGQLHHSPGEETKLEMDKFPYVALSKTYNTNAQVPDSAGTATAYLCGVKANEGTVGVSAATQRSQCNTTQGNEVTSILRWAKDAGKSVGIVTTTRVNHATPSASYAHSADRDWYSDNEMPPEALSQGCKDIAYQLMYNIKDIEVIMGGGRKYMFPKNRTDVEYELDEKARGTRLDGLNLIDIWKSFKPKHKHSHYVWNRTDLLALDPHSVDYLLGLFEPGDMQYELNRNNATDPSLSEMVEMAIRILNKNPKGFFLLVEGGRIDHGHHEGKAKQALHEAVEMDQAIGQAGAMTSVEDTLTVVTADHSHVFTFGGYTPRGNSIFGLAPMVSDTDKKPFTAILYGNGPGYKVVGGERENVSMVDYAHNNYQAQSAVPLRHETHGGEDVAVFAKGPMAHLLHGVQEQNYIPHVMAYAACIGANRDHCASASSSGSPSPGPLLLLLALLPLGSLF

SEQ ID NO: 137-151 - skipped (15 entries)

SEQ ID NO: 152 - protein, 207 aa, synthetic construct
MDKDCEMKRTTLDSPLGKLELSGCEQGLHEIKLLGKGTSAADAVEVPAPAAVLGGPEPLMQCTAWLNAYFHQPEAIEEFPVPALHHPVFQQESFTRQVLWKLLKVVKFGEVISYQQLAALAGNPKAARAVGGAMRGNPVPILIPCHRVVCSSGAVGNYSWGLAVKEWLLAHEGHRLGKPGLGGSSGLAGAWLKGAGATSGSPPAGRN

SEQ ID NO: 153 - protein, 207 aa, synthetic construct
MDKDCEMKRTTLDSPLGKLELSGCEQGLHEIKLLGKGTSAADAVEVPAPAAVLGGPEPLIQCTAWLNAYFHQPEAIEEFPVPALHHPVFQQESFTRQVLWKLLKVVKFGEVISEQQLAALVGNPKAARAVNGAMDGNPVPILIPCHRVVCSSSAVGPYLWGLAVKEWLLAHEGHRLGKPGLGGSSGLAGAWLKGAGATSGSPPAGRN

SEQ ID NO: 154-200 - skipped (47 entries)

SEQ ID NO: 201 - protein, 28 aa, synthetic construct
  SITE 22: note = Unnatural amino acid O-(2-bromoethyl)-tyrosine (O2beY)
DYKDDDDKGSGSSCWWHPQGDYQQQQQQ

SEQ ID NO: 202 - protein, 28 aa, synthetic construct
  SITE 22: note = Unnatural amino acid O-(2-bromoethyl)-tyrosine (O2beY)
DYKDDDDKGSGSSCWWHPQGDYQQQQQQ

SEQ ID NO: 203 - protein, 26 aa, synthetic construct
  SITE 13: note = Unnatural amino acid O-(2-bromoethyl)-tyrosine (O2beY)
DYKDDDDKGSGSYDSETGECQQQQQQ

SEQ ID NO: 204 - protein, 26 aa, synthetic construct
  SITE 13: note = Unnatural amino acid O-(2-bromoethyl)-tyrosine (O2beY)
DYKDDDDKGSGSYDSETGECQQQQQQ

SEQ ID NO: 205 - protein, 32 aa, synthetic construct
  SITE 17: note = Unnatural amino acid O-(2-bromoethyl)-tyrosine (O2beY)
DYKDDDDKGSGSTLSWYEAMDMCTSSQQQQQQ

SEQ ID NO: 206 - protein, 32 aa, synthetic construct; identical to SEQ ID NO: 205
  SITE 17: note = Unnatural amino acid O-(2-bromoethyl)-tyrosine (O2beY)

SEQ ID NO: 207 - protein, 32 aa, synthetic construct
  SITE 17: note = Unnatural amino acid O-(2-bromoethyl)-tyrosine (O2beY)
DYKDDDDKGSGSTLSWYEAMDMCTSSGSGSGS

SEQ ID NO: 208 - protein, 36 aa, synthetic construct
  SITE 17: note = Unnatural amino acid O-(2-bromoethyl)-tyrosine (O2beY)
DYKDDDDKGSGSTLSWYEAMDMCTSSQQQQQQIEGR

SEQ ID NO: 209 - protein, 36 aa, synthetic construct
  SITE 17: note = Unnatural amino acid p-(2-chloroacetamido)-phenylalanine ( pCaaF)
DYKDDDDKGSGSTLSWFEAMDMCTSSQQQQQQIEGR

SEQ ID NO: 210 - protein, 36 aa, synthetic construct
  SITE 17: note = Unnatural amino acid O-(2-bromoethyl)-tyrosine (O2beY)
DYKDDDDKGSGSTLSWYEAMDMCTSSGSGSGSIEGR

SEQ ID NO: 211 - protein, 31 aa, synthetic construct
  SITE 13: note = Unnatural amino acid O-(2-bromoethyl)-tyrosine (O2beY)
DYKDDDDKGSGSYVYNRMKCGQQQQQQIEGR

SEQ ID NO: 212 - protein, 16 aa, synthetic construct
  SITE 7: note = Unnatural amino acid O-(2-bromoethyl)-tyrosine (O2beY)
QGTLSWYEAMDMCTSS

SEQ ID NO: 213 - protein, 30 aa, synthetic construct
  SITE 15: note = Unnatural amino acid p-(2-chloroacetamido)-phenylalanine ( pCaaF)
DYKDDDDKGSGSWHFSFNWRWLPCQQQQQQ

SEQ ID NO: 214 - protein, 37 aa, synthetic construct
  SITE 23: note = Unnatural amino acid p-(2-chloroacetamido)-phenylalanine ( pCaaF)
DYKDDDDKGSGSTYLCGAISLAFKAQIKESLQQQQQQ

SEQ ID NO: 215 - protein, 30 aa, synthetic construct
  SITE 20: note = Unnatural amino acid p-(2-chloroacetamido)-phenylalanine ( pCaaF)
DYKDDDDKGSGSCRDRGDYFQQQQQQIEGR

SEQ ID NO: 216 - protein, 33 aa, synthetic construct
  SITE 23: note = Unnatural amino acid p-carboxymethyl-L-phenylalanine
DYKDDDDKGSGSCRDRGDYPCMFQQQQQQIEGR

SEQ ID NO: 217 - protein, 30 aa, synthetic construct
  SITE 13: note = Unnatural amino acid O-(2-bromoethyl)-tyrosine (O2beY)
DYKDDDDKGSGSYLGRGDYCQQQQQQIEGR

SEQ ID NO: 218 - protein, 30 aa, synthetic construct
  SITE 19: note = Unnatural amino acid O-(2-bromoethyl)-tyrosine (O2beY)
DYKDDDDKGSGSCLGRGDYYQQQQQQIEGR

SEQ ID NO: 219 - protein, 30 aa, synthetic construct
  SITE 13: note = Unnatural amino acid O-(2-bromoethyl)-tyrosine (O2beY)
DYKDDDDKGSGSYHVRGDNCQQQQQQIEGR

SEQ ID NO: 220 - protein, 30 aa, synthetic construct
  SITE 20: note = Unnatural amino acid O-(2-bromoethyl)-tyrosine (O2beY)
DYKDDDDKGSGSCHVRGDNYQQQQQQIEGR

SEQ ID NO: 221 - protein, 30 aa, synthetic construct
  SITE 13: note = Unnatural amino acid O-(2-bromoethyl)-tyrosine (O2beY)
DYKDDDDKGSGSYHRGDHLCQQQQQQIEGR

SEQ ID NO: 222 - protein, 30 aa, synthetic construct
  SITE 20: note = Unnatural amino acid O-(2-bromoethyl)-tyrosine (O2beY)
DYKDDDDKGSGSCHRGDHLYQQQQQQIEGR

SEQ ID NO: 223 - protein, 30 aa, synthetic construct
  SITE 13: note = Unnatural amino acid O-(2-bromoethyl)-tyrosine (O2beY)
DYKDDDDKGSGSYLGRGDNCQQQQQQIEGR

SEQ ID NO: 224 - protein, 30 aa, synthetic construct
  SITE 20: note = Unnatural amino acid O-(2-bromoethyl)-tyrosine (O2beY)
DYKDDDDKGSGSCLGRGDNYQQQQQQIEGR

SEQ ID NO: 225 - protein, 30 aa, synthetic construct
  SITE 13: note = Unnatural amino acid O-(2-bromoethyl)-tyrosine (O2beY)
DYKDDDDKGSGSYRDRGDYCQQQQQQIEGR

SEQ ID NO: 226 - protein, 30 aa, synthetic construct
  SITE 19: note = Unnatural amino acid O-(2-bromoethyl)-tyrosine (O2beY)
DYKDDDDKGSGSCRDRGDYYQQQQQQIEGR

SEQ ID NO: 227 - protein, 29 aa, synthetic construct
  SITE 13: note = Unnatural amino acid O-(2-bromoethyl)-tyrosine (O2beY)
DYKDDDDKGSGSYGRGDGCQQQQQQIEGR

SEQ ID NO: 228 - protein, 29 aa, synthetic construct
  SITE 19: note = Unnatural amino acid O-(2-bromoethyl)-tyrosine (O2beY)
DYKDDDDKGSGSCGRGDGYQQQQQQIEGR

SEQ ID NO: 229 - protein, 26 aa, synthetic construct
  SITE 12: note = Unnatural amino acid p-(2-chloroacetamido)-phenylalanine ( pCaaF)
MTYLCGAISLAFKAQIKESLQQQQQQ

SEQ ID NO: 230 - protein, 31 aa, synthetic construct
  SITE 17: note = Unnatural amino acid p-(2-chloroacetamido)-phenylalanine ( pCaaF)
HAGSGSTYLCGAISLAFKAQIKESLQQQQQQ

SEQ ID NO: 231 - protein, 37 aa, synthetic construct
  SITE 23: note = Unnatural amino acid p-(2-chloroacetamido)-phenylalanine ( pCaaF)
DYKDDDDKGSGSTYLCGAISLAFKAQIKESLGSGSGS

SEQ ID NO: 232 - protein, 36 aa, synthetic construct
  SITE 23: note = Unnatural amino acid p-(2-chloroacetamido)-phenylalanine ( pCaaF)
DYKDDDDKGSGSTYLCGAISLAFKAQIKESLPAPAP

SEQ ID NO: 233 - protein, 36 aa, synthetic construct
  SITE 23: note = Unnatural amino acid p-(2-chloroacetamido)-phenylalanine ( pCaaF)
DYKDDDDKGSGSTYLCGAISLAFKAQIKESLEAAAK

SEQ ID NO: 234 - protein, 43 aa, synthetic construct
  SITE 23: note = Unnatural amino acid p-(2-chloroacetamido)-phenylalanine ( pCaaF)
DYKDDDDKGSGSTYLCGAISLAFKAQIKESLQQQQQQQQQQQQ

SEQ ID NO: 235 - protein, 54 aa, synthetic construct
  SITE 30: note = Unnatural amino acid O-(2-bromoethyl)-tyrosine (O2beY)
  SITE 46: note = Unnatural amino acid O-(2-bromoethyl)-tyrosine (O2beY)
DYKDDDDKGSGSSCWWHPQGDYGSGSENLYFQGSGSSCWWHPQGDYIEGRGSGS

SEQ ID NO: 236 - protein, 50 aa, synthetic construct
  SITE 20: note = Unnatural amino acid O-(2-bromoethyl)-tyrosine (O2beY)
  SITE 27: note = Unnatural amino acid O-(2-bromoethyl)-tyrosine (O2beY)
DYKDDDDKGSGSYDSETGECGSGSENLYFQGSGSDSEYTGECIEGRGSGS

SEQ ID NO: 237 - protein, 58 aa, synthetic construct
  SITE 13: note = Unnatural amino acid O-(2-bromoethyl)-tyrosine (O2beY)
  SITE 32: note = Unnatural amino acid O-(2-bromoethyl)-tyrosine (O2beY)
DYKDDDDKGSGSYDSETGECQQQQQQFCFCDYKDDDDKGSGSSCWWHPQGDYQQQQQQ

SEQ ID NO: 238 - protein, 69 aa, synthetic construct
  SITE 13: note = Unnatural amino acid O-(2-bromoethyl)-tyrosine (O2beY)
  SITE 36: note = Unnatural amino acid O-(2-bromoethyl)-tyrosine (O2beY)
DYKDDDDKGSGSYDSETGECQQQIGERQQQFCFCDYKDDDDKGSGSSCWWHPQGDYQQQIGERQQQQQQ

SEQ ID NO: 239 - protein, 73 aa, synthetic construct
  SITE 27: note = Unnatural amino acid p-(2-chloroacetamido)-phenylalanine ( pCaaF)
  SITE 42: note = Unnatural amino acid p-(2-chloroacetamido)-phenylalanine ( pCaaF)
DYKDDDDKGSGSTYLCGAISLAPCAAFKAQIKESLQQQQQQFCFCDYKDDDDKGSGSSCWWHPQGDFQQQQQQ

SEQ ID NO: 240 - protein, 79 aa, synthetic construct
  SITE 23: note = Unnatural amino acid p-(2-chloroacetamido)-phenylalanine ( pCaaF)
  SITE 70: note = Unnatural amino acid p-(2-chloroacetamido)-phenylalanine ( pCaaF)
DYKDDDDKGSGSTYLCGAISLAFKAQIKESLQQQQQQFCFCDYKDDDDKGSGSSCWWHPQGDQQQQQQIFGERQQQQQQ

SEQ ID NO: 241 - protein, 100 aa, synthetic construct
  SITE 13: note = Unnatural amino acid O-(2-bromoethyl)-tyrosine (O2beY)
DYKDDDDKGSGSYHVRGDNCMKIEEGKLTNPGVSAWQVNTAYTAGQLVTYNGKTYKCLQPHTSLAGWEPSNVPALWQLQNNGNNGLELRHGHISHHHHHH

SEQ ID NO: 242 - protein, 97 aa, synthetic construct
  SITE 13: note = Unnatural amino acid p-(2-chloroacetamido)-phenylalanine ( pCaaF)
DYKDDDDKGSGSFHVRGDNCMKIEEGKLTNPGVSAWQVNTAYTAGQLVTYNGKTYKCLQPHTSLAGWEPSNVPALWQLQNNGNNGLELRHGHHHHHH

SEQ ID NO: 243 - protein, 97 aa, synthetic construct; identical to SEQ ID NO: 242
  SITE 13: note = Unnatural amino acid p-carboxymethyl-l-phenylalanine

SEQ ID NO: 244 - protein, 96 aa, synthetic construct
  SITE 41: note = Unnatural amino acid O-(2-bromoethyl)-tyrosine (O2beY)
DYKDDDDKGGHCHVRGDNYMKIEEGKLTNPGVSAWQVNTAYTAGQLVTYNGKTYKCLQPHTSLAGWEPSNVPALWQLQNNGNNGLELRHGHHHHHH

SEQ ID NO: 245 - protein, 97 aa, synthetic construct
  SITE 20: note = Unnatural amino acid p-(2-chloroacetamido)-phenylalanine ( pCaaF)
DYKDDDDKGSGSCHVRGDNFMKIEEGKLTNPGVSAWQVNTAYTAGQLVTYNGKTYKCLQPHTSLAGWEPSNVPALWQLQNNGNNGLELRHGHHHHHH

SEQ ID NO: 246 - protein, 94 aa, synthetic construct
  SITE 20: note = Unnatural amino acid p-(2-chloroacetamido)-phenylalanine ( pCaaF)
DYKDDDDKGSGSCHVRGDNFMKIEEGKLTNPGVSAWQVNTAYTAGQLVTYNGKTYKCLQPHTSLAGWEPSNVPALWQLQNNGNNGLELRHGHHH

SEQ ID NO: 247 - protein, 97 aa, synthetic construct
  SITE 50: note = Unnatural amino acid O-(2-bromoethyl)-tyrosine (O2beY)
DYKDDDDKGSGSYHRGDHLCMKIEEGKLTNPGVSAWQVNTAYTAGQLVTYNGKTYKCLQPHTSLAGWEPSNVPALWQLQNNGNNGLELRHGHHHHHH

SEQ ID NO: 248 - protein, 97 aa, synthetic construct
  SITE 13: note = Unnatural amino acid p-(2-chloroacetamido)-phenylalanine ( pCaaF)
DYKDDDDKGSGSFHRGDHLCMKIEEGKLTNPGVSAWQVNTAYTAGQLVTYNGKTYKCLQPHTSLAGWEPSNVPALWQLQNNGNNGLELRHGHHHHHH

SEQ ID NO: 249 - protein, 97 aa, synthetic construct; identical to SEQ ID NO: 248
  SITE 13: note = unnatural amino acid p-carboxymethyl-l-phenylalanine

SEQ ID NO: 250 - protein, 97 aa, synthetic construct
  SITE 20: note = Unnatural amino acid O-(2-bromoethyl)-tyrosine (O2beY)
DYKDDDDKGSGSCHRGDHLYMKIEEGKLTNPGVSAWQVNTAYTAGQLVTYNGKTYKCLQPHTSLAGWEPSNVPALWQLQNNGNNGLELRHGHHHHHH

SEQ ID NO: 251 - protein, 97 aa, synthetic construct
  SITE 20: note = Unnatural amino acid p-(2-chloroacetamido)-phenylalanine ( pCaaF)
DYKDDDDKGSGSCHRGDHLFMKIEEGKLTNPGVSAWQVNTAYTAGQLVTYNGKTYKCLQPHTSLAGWEPSNVPALWQLQNNGNNGLELRHGHHHHHH

SEQ ID NO: 252 - protein, 97 aa, synthetic construct; identical to SEQ ID NO: 251
  SITE 20: note = p-carboxymethyl-l-phenylalanine

SEQ ID NO: 253 - protein, 97 aa, synthetic construct
  SITE 13: note = Unnatural amino acid O-(2-bromoethyl)-tyrosine (O2beY)
DYKDDDDKGSGSYRDRGDYCMKIEEGKLTNPGVSAWQVNTAYTAGQLVTYNGKTYKCLQPHTSLAGWEPSNVPALWQLQNNGNNGLELRHGHHHHHH

SEQ ID NO: 254 - protein, 97 aa, synthetic construct
  SITE 13: note = Unnatural amino acid p-(2-chloroacetamido)-phenylalanine ( pCaaF)
DYKDDDDKGSGSFRDRGDYCMKIEEGKLTNPGVSAWQVNTAYTAGQLVTYNGKTYKCLQPHTSLAGWEPSNVPALWQLQNNGNNGLELRHGHHHHHH

SEQ ID NO: 255 - protein, 97 aa, synthetic construct; identical to SEQ ID NO: 254
  SITE 13: note = p-carboxymethyl-l-phenylalanine

SEQ ID NO: 256 - protein, 97 aa, synthetic construct
  SITE 20: note = Unnatural amino acid O-(2-bromoethyl)-tyrosine (O2beY)
DYKDDDDKGSGSCRDRGDYYMKIEEGKLTNPGVSAWQVNTAYTAGQLVTYNGKTYKCLQPHTSLAGWEPSNVPALWQLQNNGNNGLELRHGHHHHHH

SEQ ID NO: 257 - protein, 97 aa, synthetic construct
  SITE 20: note = Unnatural amino acid p-(2-chloroacetamido)-phenylalanine ( pCaaF)
DYKDDDDKGSGSCRDRGDYFMKIEEGKLTNPGVSAWQVNTAYTAGQLVTYNGKTYKCLQPHTSLAGWEPSNVPALWQLQNNGNNGLELRHGHHHHHH

SEQ ID NO: 258 - protein, 97 aa, synthetic construct; identical to SEQ ID NO: 257
  SITE 20: note = Unnatural amino acid p-carboxymethyl-l-phenylalanine

(19) United States
(12) Patent Application Publication
(10) Pub. No.: US 2025/0197448 A1
(43) Pub. Date: Jun. 19, 2025

(54) CYCLOPEPTIBODIES AND USES THEREOF

(71) Applicant: University of Rochester, Rochester, NY (US)

(72) Inventors: Rudi Fasan, Allen, TX (US); Yu Gu, Rockville, MD (US)

(73) Assignee: University of Rochester, Rochester, NY (US)

(21) Appl. No.: 18/845,567

(22) PCT Filed: Mar. 10, 2023
(86) PCT No.: PCT/US2023/064134

§ 371 Date: Sep. 10, 2024

Related U.S. Application Data
(60) Provisional application No. 63/318,962, filed on Mar. 11, 2022.

(51) Int. Cl. C07K 7/50; A61K 38/00

(52) U.S. Cl. CPC C07K 7/50; A61K 38/00; C07K 2319/30

(57) ABSTRACT

Methods and compositions for generating novel proteins comprise a genetically encoded macrocyclic peptide fused to an immunoglobulin Fc region, referred to as “cyclopeptibodies.” Methods and compositions are provided for making cyclopeptide-Fc region fusion proteins from genetically encoded, ribosomally produced artificial polypeptides. These methods are based on the genetic fusion of an immunoglobulin Fc region to an artificial precursor polypeptide comprising a non-canonical amino acid residue carrying a thiol-reactive functional group; and a cysteine residue that is positioned either upstream or downstream of the non-canonical amino acid in the polypeptide sequence. These methods are based on the ability of the functional group-bearing amino acid and cysteine residue to react after ribosomal synthesis of the polypeptide, so that a cyclic peptide carrying a side-chain-to-side-chain covalent (thioether) linkage is formed and that thioether-linked cyclic peptide is genetically fused to the Fc region of an immunoglobulin.

### CROSS-REFERENCE TO RELATED APPLICATIONS

This application claims the benefit of U.S. Provisional Patent Application No. 63/318,962, filed Mar. 11, 2022, which is incorporated by reference herein in its entirety.

### STATEMENT REGARDING FEDERALLY SPONSORED RESEARCH OR DEVELOPMENT

The disclosed invention was made with government support under Grant No. R01GM134076 from the National Institutes of Health. The government has rights in this invention.

### 1. TECHNICAL FIELD

The present invention relates to methods and compositions for generating proteins comprising a genetically encoded macrocyclic peptide fused to an immunoglobulin Fc region, referred herein as ‘cyclopeptibodies’. The invention relates to methods and compositions for generating cyclopeptibodies. The invention relates to nucleic acid molecules, polypeptides, methods and preparations for preparing cyclopeptibodies targeted against a protein of interest, and their use for detection, imaging, or modulation of the function of said target protein.

### 2. BACKGROUND

Immunoglobulins constitute an important and well-established class of therapeutic agents (e.g., therapeutic antibodies) as well as important protein-based reagents for imaging, detection, and sensing of an antigen molecule for a broad range of biomedical and biotechnological applications.

Immunoglobulins comprise four polypeptide chains, i.e., two heavy and two light chains associated via interchain disulfide bonds. Each light chain has two domains, namely the variable light domain (VL) and the constant light chain domain (CL), and each heavy chain has two regions, namely the variable heavy chain region (VH) and the constant heavy chain region (CH). The constant heavy chain region (CH) consists of the constant heavy chain region (e.g., CH1, CH2, CH3, etc.) designated by number (e.g., U.S. Pat. No. 6,086,875 (Blumberg R S, et al.), U.S. Pat. No. 5,624,821 (Winter G P et al.) and U.S. Pat. No. 5,116,964 (Capon D J and Lasky L A)). Immunoglobulins are classified into different isotypes (i.e., IgG, IgM, IgA, IgD and IgE) based on their biological properties, their location in the organism and their ability to process different antigens.

Depending on the immunoglobulin isotype, the constant heavy chain region (CH) may have three or four CH domains. In addition, in some isotypes (IgA, IgD and IgG), the heavy chain contains a hinge region that adds flexibility to the molecule (Janeway et al. 2001, Immunobiology, Garland Publishing, N.Y., N.Y.).

In humans there are four IgG subclasses (IgG1, 2, 3, 4), which are named according to the order in which they are abundant in serum, with IgG1 being the most abundant. The IgG isotype consists of two light chains and two heavy chains, each heavy chain comprising three constant heavy chain domains (CH1, CH2, CH3). The two heavy chains of IgG are linked by disulfide bonds (—S—S—) to each other and to the light chain, respectively. The antigen binding site of IgG is located in a fragment antigen binding region (Fab region) comprising the variable light (VL) and variable heavy (VH) domains as well as the constant light (CL) and constant heavy (CH1) domains. The fragment crystallizable region (Fc region) of IgG is part of a heavy chain that contains CH2 and CH3 domains that bind Fc receptors found on the surface of certain cells, including neonatal Fc receptors (FcRn). The heavy chain of IgG also has a hinge region (hinge) between CH1 and CH2, which separates the Fab region from the Fc region and joins the two heavy chains together via disulfide bonds.

The Fc region is primarily responsible for mediating many effector functions of immunoglobulins through its interactions with Fc receptors on the surface of various cells of the innate or adaptive immune system, including, among others, B lymphocytes, natural killer cells, dendritic cells, macrophages, neutrophils, eosinophils, basophils, human platelets, and mast cells. In addition, the Fc region of immunoglobulins (e.g., IgGs) can interact with the neonatal Fc receptor (FcRn), generally expressed in placental and epithelial cells, and this interaction contributes to the preservation of immunoglobulins via the endocytosis salvage pathway and to the extended serum half-life properties of antibodies in an organism.

The immunoglobulin Fc domain has found widespread use as a carrier protein for a variety of therapeutic and diagnostic molecules. When constructed together with a therapeutic protein or peptide, an Fc domain can provide longer in vivo half-life, or can incorporate such functions as Fc receptor binding, protein A binding, complement fixation and perhaps even placental transfer. As an example, the FDA-approved drug Etanercept (Enbrel®) used for the treatment of treat rheumatoid arthritis, consists of a TNF receptor fused to the constant domain of an antibody. Other examples of protein-Fc fusion constructs investigated for therapeutic applications that are known in the art include Fc fusions with CD30-L (U.S. Pat. No. 5,480,981), IL-10 (Zheng et al., (1995), J Immunol. 154:5590-600), CD4 receptor (Capon et al. (1989), Nature 337:525-31), IL-2 (Harvill et al. (1995), Immunotech. 1:95-105), and CTL-4 (Linsley (1991), J. Exp. Med. 174:561-9).

A more recent development in this field are Fc-peptide fusions known as “peptibodies”, which consists of a Fc region fused at its N-terminus or C-terminus to a linear or disulfide-bridged peptide. Examples of peptibodies known in the art are, for example, described in U.S. Pat. No. 6,660,843, U.S. Pat. App. No. 2003/0176352; U.S. Ser. No. 09/422,838; U.S. Pat. App. No. 2003/0229023, U.S. Pat. App. No. 2003/0236193; U.S. Ser. No. 10/666,480, U.S. Patent App. No. 2006/0140934.

Despite this progress, a major disadvantage of peptibodies is that, similar to linear peptides, bioactive linear peptides contained in these constructs are prone to proteolytic degradation and/or may fail to bind proteins that recognize discontinuous epitopes. While bioactive peptides containing disulfide bridges have also been investigated, disulfide bonds are easily cleaved under reducing conditions and may result in disulfide scrambling (e.g., with other disulfide bridges present in the immunoglobulin molecules), which can compromise the function of these molecules and/or complicate their production via recombinant expression.

Compared to linear peptides, cyclic peptides constrained by stable intramolecular linkages present several advantages, including enhanced enzymatic stability (Fairlie et al. 2000; Wang, Liao, and Arora 2005), membrane permeability (Walensky et al. 2004; Rezai, Bock, et al. 2006; Rezai, Yu, et al. 2006), and protein binding affinity (Tang et al. 1999; Dias et al. 2006) and selectivity (Henchey et al. 2010). Constraints that lock-in the active conformation of a peptide molecule can result in increased affinity due to the reduced conformational entropy loss upon binding to the receptor. Many bioactive and therapeutically relevant peptides isolated from natural sources occur indeed in cyclized form or contain intramolecular linkages that reduce the conformational flexibility of these molecules (e.g., immunosuppressant cyclosporin A, antitumor dolastatin 3 and diazonamide A, anti-HIV luzopeptin E2, and the antimicrobial vancomycin).

While cyclic peptide-antibody conjugates can be prepared via bioconjugation of a cyclic peptide to an antibody fragment or, alternatively, by chemical crosslinking of a linear peptide with cysteine-reactive cross-linking agent (e.g., Angelini et al., Bioconjug Chem, 2012 Sep. 19; 23(9):1856-63), these methods are laborious and require additional manipulation of the antibody molecule. Furthermore, these chemical treatments are known to result in varying degrees of heterogeneity of the bioconjugate, are typically low yielding, and can result in undesired reactions with loss in functional activity.

The art would benefit from a technology that enable the streamlined generation and production of Fc-cyclic peptide fusions for use as therapeutic agents and other biomedical and biotechnological applications.

Citation or identification of any reference in Section 2, or in any other section of this application, shall not be considered an admission that such reference is available as prior art to the present invention.

### 4. BRIEF DESCRIPTION OF THE DRAWINGS

Embodiments are described herein with reference to the accompanying drawings, in which similar reference characters denote similar elements throughout the several views. It is to be understood that in some instances, various aspects of the embodiments may be shown exaggerated or enlarged to facilitate an understanding of the invention.

FIG. 1 is a schematic representation of a general method for making cyclopeptibody molecule from precursor polypeptides of general formula (I) within the invention. An Fc domain is comprised within the N-terminal tail (AA)m, or within the C-terminal tail (AA)p, or both. W corresponds to the linker group resulting from the bond-forming reaction between the functional group FG₁ and the cysteine residue.

FIG. 2 is a schematic representation of a general method for making cyclopeptibody molecule from precursor polypeptides of general formula (II) within the invention. An Fc domain is comprised within the N-terminal tail (AA)m, or within the C-terminal tail (AA)p, or both. W corresponds to the linker group resulting from the bond-forming reaction between the functional group FG₁ and the cysteine residue.

FIG. 3 is a schematic representation of a cyclopeptibody comprising a cyclic peptide fused to the N-terminus of an Fc domain (FIG. 3A) or fused to the C-terminus of an Fc domain (FIG. 3B), as prepared according to the methods of the invention.

FIG. 4 is a schematic representation of a polyvalent cyclopeptibody molecule (FIG. 4A) and bispecific cyclopeptibody molecule (FIG. 4B), as prepared according to the methods of the invention.

FIG. 5 shows synthetic routes for the synthesis of the cysteine-reactive unnatural amino acids p-2beF (FIG. 5A), 2becK (FIG. 5B), and p-1beF (FIG. 5C).

FIG. 6 shows synthetic routes for the synthesis of the cysteine-reactive unnatural amino acids 2cecK (FIG. 6A), bdnK (FIG. 6B), and OdbpY (FIG. 6C).

FIG. 7 shows synthetic routes for the synthesis of the cysteine-reactive unnatural amino acids pVsaF (FIG. 7A) and pAaF (FIG. 7B).

FIG. 8 shows synthetic routes for the synthesis of the cysteine-reactive unnatural amino acids pCaaF (FIG. 8A), O4bbeY (FIG. 8B), and pCmF (FIG. 8C).

FIG. 9 illustrates the identification of a suitable orthogonal AARS/tRNA pairs for the incorporation of the non-canonical amino acid pCmF using a fluorescence-based assay. FIG. 9A depicts the relative efficiency of incorporation of the unnatural amino acid pCmF into the reporter protein YFP (TAG) via amber codon suppression using a panel of engineered Mj-TyrRS variants in combination with the cognate amber stop codon suppressor tRNA Mj-tRNA^{Tyr}_{CUA}. FIG. 9B depicts a series of target sequences used to assess the cyclization efficiency of the pCmF amino acid via proximity-induced cysteine alkylation. All these constructs were determined to have undergone efficient cyclization (>90-95% yield) upon expression in E. coli in a model CBD-fusion construct.

FIG. 10 shows purification and characterization of representative cyclopeptibody molecules. FIG. 10A is an SDS PAGE gel analysis of IgG1 Fc domain (Tras-Fc), Keap1-binding cyclopeptibody KKD1-Tras-Fc, and streptavidin-binding cyclopeptibody Strep-m3-Tras-Fc. FIG. 10B consists of MALDI-TOF spectra of PD-L1-binding cyclopeptibody RK10-Tras-Fc-His and Keap1-binding cyclopeptibody KKD1-Tras-Fc. FIG. 10C is a gel permeation chromatogram of Keap1-binding cyclopeptibody KKD1-Tras-Fc-His purified via protein G vs. Ni-affinity chromatography.

FIG. 11 shows a streptavidin-binding cyclopeptibody. FIG. 11A is a schematic representation of cyclopeptibody Strep-m3-Tras-Fc. FIG. 11B shows binding curves for cyclopeptibody Strep-m3-Tras-Fc, cyclic peptide Strep-m3 alone (as CDB fusion), and Fc domain alone in the streptavidin-binding assay.

FIG. 12 shows a Keap1-binding cyclopeptibody. FIG. 12A is a schematic representation of cyclopeptibody KKD-m1-Tras-Fc. FIG. 12B shows binding curves for cyclopeptibody KKD-m1-Tras-Fc, cyclic peptide KKD-m1 alone (as CDB fusion), and Fc domain alone in the Keap1-binding assay.

FIG. 13 shows a Hedgehog-binding cyclopeptibody. FIG. 13A shows a schematic representation of cyclopeptibody L2-m5-Tras-Fc. FIG. 13B shows binding curves for cyclopeptibody L2-m5-Tras-Fc, cyclic peptide L2-m5 alone (as CDB fusion), and Fc domain alone in the Shh-binding assay. FIG. 13C shows binding curves for cyclopeptibody L2-m5-Tras-Fc with immobilized Shh, Ihh, or Dhh.

FIG. 14 shows the sequence and structure of PD-L1-binding linear and cyclic peptides with corresponding affinity (K_{D}) values as determined via the in vitro PD-L1 binding assay.

FIG. 15 shows PD-L1-binding cyclopeptibodies. FIG. 15A shows binding curves for cyclopeptibody cRK10 (pCaaF)-Tras-Fc and the cyclic peptide cRK10 (pCaaF) as determined via the in vitro PD-L1 binding assay. FIG. 15B shows binding curves for cyclopeptibody cCLP003 (pCaaF)-Tras-Fc and the cyclic peptide cCLP003 (pCaaF) as determined via the in vitro PD-L1 binding assay.

FIG. 16 addresses a PD-L1/PD-1 inhibition assay. The graph shows the inhibition curve for cyclopeptibody cCLP003 (pCaaF)-Tras-Fc in the competition assay with plate-immobilized in the presence of a fixed concentration of PD-1.

FIG. 17 shows a flow cytometry analysis of MDA-MB-231 cells (breast cancer) after treatment with cyclopeptibody cRK10 (pCaaF)-Tras-Fc or cCLP003 (pCaaF)-Tras-Fc, followed by secondary labeling with FITC-conjugated anti-IgG1 Fc antibody. Treatment with either cyclopeptibodies results in selective labeling of PD-L1 receptors on the cells, whereas the Fc domain alone and secondary antibody show negligible non-specific interaction. FIG. 17A shows cell count distribution vs. fluorescence intensity signal, whereas FIG. 17B reports the median fluorescence intensity (MFI) values for these data. FIG. 17C shows confocal microscopy images of MDA cells treated with cRK10 (pCaaF)-Tras-Fc under 10× (upper panels) and 60× objective lens (bottom panels). Nuclei were stained with Hoechst 33342, whereas the bound cRK10 (pCaaF)-Tras-Fc cyclopeptide was detected and stained with fluorescein-conjugated anti-human Fc antibody.

FIG. 18 shows a flow cytometry analysis of MDA-MB-231 cells (breast cancer) after treatment with cyclopeptibody cRGD5R (pCmF)-Tras-Fc or cRGD5R (pCaaF)-Tras-Fc, followed by secondary labeling with FITC-conjugated anti-IgG1 Fc antibody. Treatment with either cyclopeptibody results in selective labeling of the integrin receptors on the cells, exhibiting superior receptor affinity and labeling efficiency compared to the corresponding cyclic peptides, whose binding was detected using anti-FLAG antibody. The Fc domain alone and secondary antibody show negligible non-specific interaction. FIG. 18A shows cell count distribution vs. fluorescence intensity signal, whereas FIG. 18B reports the median fluorescence intensity (MFI) values for these data.

FIG. 19 is a proteolytic stability assay with FIG. 19A (cCLP003 (pCaaF)-Tras-Fc) and FIG. 19B (L2-m5-Tras-Fc) in human blood serum. The graphs report the amount of active cyclopeptide at different time points as measured using the in vitro PD-L1 binding assay for cCLP003 (pCaaF)-Tras-Fc and the in vitro Shh binding assay for L2-m5-Tras-Fc.

FIG. 20 shows PD-L1-targeting cyclopeptibodies with variable spacer sequences. FIG. 20A is a schematic representation of PD-L1 targeting cyclopeptibodies with various spacer sequences between the cyclic peptide and Fc domain. FIG. 20B shows binding curves of FLAG-CRK10-Q12-Tras-Fc-His, FLAG-cRK10-EAAAK-Tras-Fc-His, FLAG-cRK10-PAPAP-Tras-Fc-His, and FLAG-cRK10-(GS) 3-Tras-Fc-His as determined via the in vitro PD-L1 binding assay.

FIG. 21 shows streptavidin and keap-1 targeting polyvalent cyclopeptibodies. FIG. 21A is a schematic representation of polyvalent cyclopeptibodies. FIG. 21B shows a binding curve of FLAG-Strep-m3-Strep-m3-Tras-Fc-His targeting streptavidin as determined via the in vitro streptavidin binding assay. FIG. 21C shows a binding curve of FLAG-KKD-m1-KKD-m1-Tras-Fc-His targeting Keap-1 as determined via the in vitro Keap1 binding assay.

FIG. 22 shows bispecific cyclopeptibodies. FIG. 22A shows a schematic representation of bispecific cyclopeptibodies. FIG. 22B shows a bispecific cyclopeptibody peptide, spacer, ncAA used for cyclization, and target for each construct. FIG. 22C is an SDS-PAGE gel of recombinantly expressed bispecific cyclopeptibodies after purification by Ni-NTA.

### 5. DETAILED DESCRIPTION

For clarity of disclosure, and not by way of limitation, the detailed description is divided into the subsections set forth below.

### 5.1 Definitions

Unless defined otherwise herein, all technical and scientific terms used herein have the same meaning as commonly understood by one of ordinary skill in the art to which the disclosure pertains.

“Antibody” or “antibody molecule” or “immunoglobulin” refer to an intact antibody, or a binding fragment thereof that competes with the intact antibody for specific binding and includes chimeric, humanized, fully human, and bispecific antibodies.

In the present invention, “immunoglobulin Fc region”, “Fc region”, “immunoglobulin Fc domain”, or “Fc domain” refer to a molecule or sequence comprising the sequence of a non-antigen-binding fragment resulting from digestion of whole antibody, whether in monomeric or multimeric form. The immunoglobulin source of the Fc domain is preferably of human origin and may be any of the immunoglobulins, including IgG, IgA, IgD, IgE, and IgM.

The terms “Fc domain” or “Fc region” encompass native Fc domains and Fc domain variant molecules and sequences as defined herein. In some embodiments, the Fc domain is a native Fc domain. In other embodiments, the Fc domain can be a Fc domain variant, an analog, a mutant, a truncation, or a derivative of human Fc domain or of an alternative Fc domain polypeptide.

The Fc domains are made up of monomeric polypeptides that may be linked into dimeric or multimeric forms by covalent (i.e., disulfide bonds) and non-covalent association. The number of intermolecular disulfide bonds between monomeric subunits of Fc domain molecules ranges from 1 to 4 depending on the immunoglobulin class (e.g., IgG, IgA, IgE) or subclass (e.g., IgG1, IgG2, IgG3, IgA1, IgGA2). One example of a Fc domain is a disulfide-bonded dimer resulting from papain digestion of an IgG (see Ellison et al. (1982), Nucleic Acids Res. 10:4071-9). The term “Fc domain” as used herein is generic to the monomeric, dimeric, and multimeric forms.

The term “hybrid Fc domain” refers to a non-native Fc domain that is made up as a composition of heavy chain constant region 2 (CH2), heavy chain constant region 3 (CH3), and hinge from immunoglobulins of different classes and/or subclasses.

The term “dimer” as applied to Fc domains or molecules comprising Fc domains refers to molecules having two polypeptide chains associated covalently or non-covalently. The term “multimer” as applied to Fc domains or molecules comprising Fc domains refers to molecules having two or more polypeptide chains associated covalently, noncovalently, or by both covalent and non-covalent interactions. IgG molecules typically form dimers; IgM, pentamers; IgD, dimers; and IgA, monomers, dimers, trimers, or tetramers. Multimers may be formed by exploiting the sequence and resulting activity of the native immunoglobulin source of the Fc domain or by derivatizing such Fc domain.

In the present invention, the term “cyclopeptibody” refers to a cyclopeptide-Fc domain fusion protein obtained from a precursor polypeptide of formula (I), (II), or (V).

The singular forms “a,” “an,” and “the” used herein include plural referents unless the content clearly dictates otherwise.

The term “plurality” refers to two or more referents unless the content clearly dictates otherwise. The term “at least one” refers to one or more referents.

The term “functional group” as used herein refers to a contiguous group of atoms that, together, may undergo a chemical reaction under certain reaction conditions. Examples of functional groups are, among many others, —OH, —NH₂, —SH, —(C═O)—, —N₃, —C≡CH.

The term “aliphatic” or “aliphatic group” as used herein means a straight or branched C₁₋₁₅ hydrocarbon chain that is completely saturated or that contains at least one unit of unsaturation, or a monocyclic C₃₋₈ hydrocarbon, or bicyclic C₈₋₁₂ hydrocarbon that is completely saturated or that contains at least one unit of unsaturation, but which is not aromatic (also referred to herein as “cycloalkyl”). For example, suitable aliphatic groups include, but are not limited to, linear or branched alkyl, alkenyl, alkynyl groups or hybrids thereof such as (cycloalkyl)alkyl, (cycloalkenyl)alkyl, or (cycloalkynyl)alkyl. The alkyl, alkenyl, or alkynyl group may be linear, branched, or cyclic and may contain up to 15, up to 8, or up to 5 carbon atoms. Alkyl groups include, but are not limited to, methyl, ethyl, propyl, cyclopropyl, butyl, cyclobutyl, pentyl, and cyclopentyl groups. Alkenyl groups include, but are not limited to, propenyl, butenyl, and pentenyl groups. Alkynyl groups include, but are not limited to, propynyl, butynyl, and pentynyl groups.

The term “aryl” and “aryl group” as used herein refers to an aromatic substituent containing a single aromatic or multiple aromatic rings that are fused together, directly linked, or indirectly linked (such as linked through a methylene or an ethylene moiety). An aryl group may contain from 5 to 24 carbon atoms, 5 to 18 carbon atoms, or 5 to 14 carbon atoms.

The terms “heteroatom” means nitrogen, oxygen, or sulfur, and includes, but is not limited to, any oxidized forms of nitrogen and sulfur, and the quaternized form of any basic nitrogen. Heteroatom further includes, but is not limited to, Se, Si, or P.

The term “heteroaryl” as used herein refer to an aryl group in which at least one carbon atom is replaced with a heteroatom. In various embodiments, a heteroaryl group is a 5- to 18-membered, a 5- to 14-membered, or a 5- to 10-membered aromatic ring system containing at least one heteroatom selected from the group consisting of oxygen, sulfur, and nitrogen atoms. Heteroaryl groups include, but are not limited to, pyridyl, pyrrolyl, furyl, thienyl, indolyl, isoindolyl, indolizinyl, imidazolyl, pyridonyl, pyrimidyl, pyrazinyl, oxazolyl, thiazolyl, purinyl, quinolinyl, isoquinolinyl, benzofuranyl, and benzoxazolyl groups.

A heterocyclic group may be any monocyclic or polycyclic ring system which contains at least one heteroatom and may be unsaturated or partially or fully saturated. The term “heterocyclic” thus includes, but is not limited to, heteroaryl groups as defined above as well as non-aromatic heterocyclic groups. In various embodiments, a heterocyclic group is a 3- to 18-membered, a 3- to 14-membered, or a 3- to 10-membered, ring system containing at least one heteroatom selected from the group consisting of oxygen, sulfur, and nitrogen atoms. Heterocyclic groups include, but are not limited to, the specific heteroaryl groups listed above as well as pyranyl, piperidinyl, pyrrolidinyl, dioaxanyl, piperazinyl, morpholinyl, thiomorpholinyl, morpholinosulfonyl, tetrahydroisoquinolinyl, and tetrahydrofuranyl groups.

A halogen atom may be a fluorine, chlorine, bromine, or iodine atom.

By “optionally substituted”, it is intended that in the any of the chemical groups listed above (e.g., alkyl, alkenyl, alkynyl, heteroalkyl, heteroalkenyl, heteroalkynyl, aryl, heteroaryl, heterocyclic, triazolyl groups), at least one of the hydrogen atoms is optionally replaced with an atom or chemical group other than hydrogen. Specific examples of such substituents include, but are not limited to, halogen atoms, hydroxyl (—OH), sulfhydryl (—SH), substituted sulfhydryl, carbonyl (—CO—), carboxy (—COOH), amino (—NH₂), nitro (—NO₂), sulfo (—SO₂—OH), cyano (—C≡N), thiocyanato (—S—C═N), phosphono (—P(O)OH₂), alkyl, alkenyl, alkynyl, heteroalkyl, heteroalkenyl, heteroalkynyl, aryl, heteroaryl, heterocyclic, alkylthiol, alkyloxy, alkylamino, arylthiol, aryloxy, or arylamino groups. Where “optionally substituted” modifies a series of groups separated by commas (e.g., “optionally substituted A, B, or C”; or “A, B, or C optionally substituted with”), it is intended that each of the groups (e.g., A, B, or C) is optionally substituted.

The term “heteroatom-containing aliphatic” as used herein refer to an aliphatic moiety where at least one carbon atom is replaced with a heteroatom, e.g., oxygen, nitrogen, sulfur, selenium, phosphorus, or silicon, and typically oxygen, nitrogen, or sulfur.

The terms “alkyl” and “alkyl group” as used herein refer to a linear, branched, or cyclic saturated hydrocarbon typically containing 1 to 24 carbon atoms, or 1 to 12 carbon atoms, such as methyl, ethyl, n-propyl, isopropyl, n-butyl, isobutyl, t-butyl, octyl, decyl and the like.

The term “heteroatom-containing alkyl” as used herein refers to an alkyl moiety where at least one carbon atom is replaced with a heteroatom, e.g., oxygen, nitrogen, sulfur, phosphorus, or silicon, and typically oxygen, nitrogen, or sulfur.

The terms “alkenyl” and “alkenyl group” as used herein refer to a linear, branched, or cyclic hydrocarbon group of 2 to 24 carbon atoms, or of 2 to 12 carbon atoms, containing at least one double bond, such as ethenyl, n-propenyl, isopropenyl, n-butenyl, isobutenyl, octenyl, decenyl, and the like.

The term “heteroatom-containing alkenyl” as used herein refer to an alkenyl moiety where at least one carbon atom is replaced with a heteroatom.

The terms “alkynyl” and “alkynyl group” as used herein refer to a linear, branched, or cyclic hydrocarbon group of 2 to 24 carbon atoms, or of 2 to 12 carbon atoms, containing at least one triple bond, such as ethynyl, n-propynyl, and the like.

The term “heteroatom-containing alkynyl” as used herein refer to an alkynyl moiety where at least one carbon atom is replaced with a heteroatom.

The term “heteroatom-containing aryl” as used herein refer to an aryl moiety where at least one carbon atom is replaced with a heteroatom.

The terms “alkoxy” and “alkoxy group” as used herein refer to an aliphatic group or a heteroatom-containing aliphatic group bound through a single, terminal ether linkage. In various embodiments, aryl alkoxy groups contain 1 to 24 carbon atoms, or contain 1 to 14 carbon atoms.

The terms “aryloxy” and “aryloxy group” as used herein refer to an aryl group or a heteroatom-containing aryl group bound through a single, terminal ether linkage. In various embodiments, aryloxy groups contain 5 to 24 carbon atoms, or contain 5 to 14 carbon atoms.

The term “substituent” refers to a contiguous group of atoms. Examples of “substituents” include, but are not limited to: alkoxy, aryloxy, alkyl, heteroatom-containing alkyl, alkenyl, heteroatom-containing alkenyl, alkynyl, heteroatom-containing alkynyl, aryl, heteroatom-containing aryl, alkoxy, heteroatom-containing alkoxy, aryloxy, heteroatom-containing aryloxy, halo, hydroxyl (—OH), sulfhydryl (—SH), substituted sulfhydryl, carbonyl (—CO—), thiocarbonyl, (—CS—), carboxy (—COOH), amino (—NH₂), substituted amino, nitro (—NO₂), nitroso (—NO), sulfo (—SO₂—OH), cyano (—C═N), cyanato (—O—C═N), thiocyanato (—S—C═N), formyl (—CO—H), thioformyl (—CS—H), phosphono (—P(O)OH₂), substituted phosphono, and phospho (—PO₂).

The term “contact” as used herein with reference to interactions of chemical units indicates that the chemical units are at a distance that allows short range non-covalent interactions (such as Van der Waals forces, hydrogen bonding, hydrophobic interactions, electrostatic interactions, dipole-dipole interactions) to dominate the interaction of the chemical units. For example, when a protein is ‘contacted’ with a chemical species, the protein is allowed to interact with the chemical species so that a reaction between the protein and the chemical species can occur.

The term “bioorthogonal” as used herein with reference to a reaction, reagent, or functional group, indicates that such reaction, reagent, or functional group does not exhibit significant or detectable reactivity towards biological molecules such as those present in a bacterial, yeast or mammalian cell. The biological molecules can be, e.g., proteins, nucleic acids, fatty acids, or cellular metabolites.

In general, the term “mutant” or “variant” as used herein with reference to a molecule such as polynucleotide or polypeptide, indicates that such molecule has been mutated from the molecule as it exists in nature. In particular, the term “mutate” and “mutation” as used herein indicates any modification of a nucleic acid and/or polypeptide which results in an altered nucleic acid or polypeptide. Mutations include, but are not limited to, any process or mechanism resulting in a mutant protein, enzyme, polynucleotide, or gene. A mutation can occur in a polynucleotide or gene sequence, by point mutations, deletions, or insertions of single or multiple nucleotide residues. A mutation in a polynucleotide includes, but is not limited to, mutations arising within a protein-encoding region of a gene as well as mutations in regions outside of a protein-encoding sequence, such as, but not limited to, regulatory or promoter sequences. A mutation in a coding polynucleotide such as a gene can be “silent”, i.e., not reflected in an amino acid alteration upon expression, leading to a “sequence-conservative” variant of the gene. A mutation in a polypeptide includes, but is not limited to, mutation in the polypeptide sequence and mutation resulting in a modified amino acid. Non-limiting examples of a modified amino acid include, but are not limited to, a glycosylated amino acid, a sulfated amino acid, a prenylated (e.g., farnesylated, geranylgeranylated) amino acid, an acetylated amino acid, an acylated amino acid, a PEGylated amino acid, a biotinylated amino acid, a carboxylated amino acid, a phosphorylated amino acid, and the like.

The term “engineer” refers to any manipulation of a molecule that result in a detectable change in the molecule, wherein the manipulation includes, but is not limited to, inserting a polynucleotide and/or polypeptide heterologous to the cell and mutating a polynucleotide and/or polypeptide native to the cell.

The term “artificial” refers to any object that is made or produced by human beings rather than occurring naturally.

The term “nucleic acid molecule” as used herein refers to any chain of at least two nucleotides bonded in sequence. For example, a nucleic acid molecule can be a DNA or an RNA.

The term “peptide”, “polypeptide”, and “protein” as used herein refers to any chain of at least two amino acids bonded in sequence, regardless of length or post-translational modification.

The. term “peptide-containing molecule” as used herein refers to a molecule that contains at least two amino acids.

The term “non-natural” and “unnatural” as used herein means being directly or indirectly made or caused to be made through human action. Thus, a “non-natural amino acid” is an amino acid that has been produced through human manipulation and does not occur in nature. The term “non-canonical amino acid” is equivalent in meaning to the terms “non-natural amino acid” or “unnatural amino acid”.

The term “cyclic” and “macrocyclic” as used herein means having constituent atoms forming a ring. Thus, a “macrocyclic peptide” is a peptide molecule that contains at least one ring formed by atoms comprised in the molecule. As such, the term “macrocyclic peptide” comprises peptides that contain at least two rings separated from each other via a polypeptide sequence (also referred to herein as “polycyclic peptides”) and peptides that contain at least two rings fused to each other (also referred to herein as “polycyclic peptides”). The term “macrocyclic peptide” also comprises peptides that contain two rings fused to each other (referred to herein also as “bicyclic peptides”).

The terms “cyclization” or “macrocyclization” as used herein refer to a process or reaction whereby a cyclic molecule is formed or is made to be formed.

The term “peptidic backbone” as used herein refers to a sequence of atoms corresponding to the main backbone of a natural protein.

The term “precursor polypeptide” or “polypeptide precursor” as used herein refers to a polypeptide that is capable of undergoing macrocyclization according to the methods disclosed herein.

The term “ribosomal polypeptide”, “ribosomally produced polypeptide” or “ribosomally derived polypeptide” as used herein refers to a polypeptide that is produced by action of a ribosome, and specifically, by the ribosomal translation of a messenger RNA encoding for such polypeptide. The ribosome can be a naturally occurring ribosome, e.g., a ribosome derived from an archea, procaryotic or eukaryotic organism, or an engineered (i.e., non-naturally occurring, artificial or synthetic) variant of a naturally occurring ribosome.

The term “affinity tag” as used herein refers to a polypeptide that is able to bind reversibly or irreversibly to an organic molecule, a metal ion, a protein, or a nucleic acid molecule.

The terms “vector” and “vector construct” as used herein refer to a vehicle by which a DNA or RNA sequence (e.g., a foreign gene) can be introduced into a host cell to transform the host and promote expression (e.g., transcription and translation) of the introduced sequence. A common type of vector is a “plasmid”, which generally is a self-contained molecule of double-stranded DNA that can be readily accept additional (foreign) DNA and which can readily be introduced into a suitable host cell. A large number of vectors, including plasmid and fungal vectors, have been described for replication and/or expression in a variety of eukaryotic and prokaryotic hosts. Non-limiting examples include, but are not limited to, pKK plasmids (Clonetech), pUC plasmids, pET plasmids (Novagen, Inc., Madison, Wis.), pRSET or pREP plasmids (Invitrogen, San Diego, Calif.), or pMAL plasmids (New England Biolabs, Beverly, Mass.), and many appropriate host cells, using methods disclosed or cited herein or otherwise known to those skilled in the relevant art. Methanococcus jannaschii tyrosyl-tRNA synthetase variant.

The terms “express” and “expression” refer to allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as a protein. The expression product itself, e.g., the resulting protein, may also be said to be “expressed” by the cell. A polynucleotide or polypeptide is expressed recombinantly, for example, when it is expressed or produced in a foreign host cell under the control of a foreign or native promoter, or in a native host cell under the control of a foreign promoter.

The term “fused” as used herein means being connected through at least one covalent bond. The term “bound” as used herein means being connected through non-covalent interactions. Examples of non-covalent interactions are van der Waals, hydrogen bond, electrostatic, and hydrophobic interactions. Thus, a “DNA-binding peptide” refers to a peptide capable of connecting to a DNA molecule via non-covalent interactions. The term “tethered” as used herein means being connected through non-covalent interactions or through covalent bonds. Thus, a “polypeptide tethered to a solid support” refers to a polypeptide that is connected to a solid support (e.g., surface, resin bead) either via non-covalent interactions or through covalent bonds.

The term “library” refers to a collection or “plurality” of at least two particles or molecules which differ in at least part of their compositions, properties, and/or sequences. For example, a macrocyclic peptide library refers to a collection of macrocyclic peptides which differs in at least part of their compositions such as, for example, an amino acid residue.

In the context of the present application, the term “desired property” refers to a predetermined property which forms the basis for the screening and/or selection of a library, such as a library of macrocyclic peptides. Such properties include but are not limited to, binding to a target molecule, blocking the function of a target molecule, blocking or promoting the interaction between a target molecule and another molecule, activating or inhibiting a reaction mediated by a target molecule, and activating or inhibiting the activity of an enzyme or receptor.

### 5.2 Methods for Generating Cyclopeptibodies

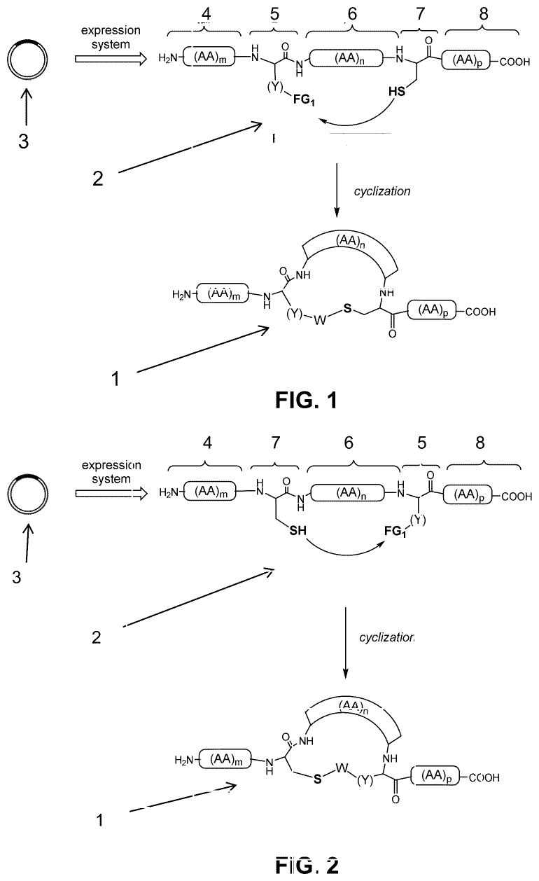

The present invention relates to novel fusion proteins comprising a cyclic peptide constrained by an intramolecular thioether linkage and an immunoglobulin Fc region. Furthermore, methods and compositions are provided for making cyclopeptide-Fc region fusion proteins from genetically encoded, ribosomally produced artificial polypeptides. These methods are based on the genetic fusion of an immunoglobulin Fc region to an artificial precursor polypeptide comprising (a) a non-canonical amino acid residue carrying a thiol-reactive functional group (referred to as FG₁); and (b) a cysteine residue that is positioned either upstream or downstream of the non-canonical amino acid in the polypeptide sequence. These methods are based on the ability of the FG₁-bearing amino acid and cysteine residue to react with each other after ribosomal synthesis of the polypeptide, so that (i) a cyclic peptide carrying a side-chain-to-side-chain covalent (thioether) linkage is formed and (ii) said thioether-linked cyclic peptide is genetically fused to the Fc region of an immunoglobulin. Schematic representations of these embodiments are provided in FIGS. 1-4. Artificial, engineered and recombinant nucleic acid molecules and peptide sequences (or amino acid sequences) for use in these methods are also provided.

In some embodiments, a method is provided for making an artificial polypeptide, the method comprising:
a. providing a nucleic acid molecule encoding for a polypeptide of structure:

(AA)ₘ-Z-(AA)ₙ-Cys-(AA)ₚ(I)

or

(AA)ₘ-Cys-(AA)ₙ-Z-(AA)ₚ (II)
wherein:
i. (AA)ₘ is an N-terminal amino acid or peptide sequence,
ii. Z is a non-canonical amino acid carrying a side-chain functional group FG₁, this FG₁ being a functional group selected from the group consisting of: —(CH₂)ₙX, where X is F, Cl, Br, or I and n is an integer number from 1 to 10; —C(O)CH₂X; where X is F, Cl, Br, or I; —CH(R′)X, where X is F, Cl, Br, or I; —C(O)CH(R′)X, where X is F, Cl, Br, or I; —OCH₂CH₂X, where X is F, Cl, Br, or I; —C(O)CH═C═C(R′)(R″); —SO₂C(R′)═C(R′)(R″); —C(O)C(R′)═C(R′)(R″); —C(R′)═C(R′)C(O)OR′; —C(R′)═C(R′)C(O)N(R′)(R″); —C(R′)═C(R′)—CN; —C(R′)═C(R′)—NO₂; —C≡C—C(O)OR′; —C≡C—C(O)N(R′)(R″); unsubstituted or substituted oxirane; unsubstituted or substituted aziridine; 1,2-oxathiolane 2,2-dioxide; 4-fluoro-1,2-oxathiolane 2,2-dioxide; and 4,4-difluoro-1,2-oxathiolane 2,2-dioxide, where each R′ and R″ is independently H, an aliphatic, a substituted aliphatic, an aryl, or a substituted aryl group.
iii. (AA)ₙ is a target peptide sequence,
iv. (AA)ₚ is a C-terminal amino acid or peptide sequence, and
vii. at least one of (AA)ₚ and (AA)ₘ comprises the Fc region of an immunoglobulin molecule or fragment thereof;
b. introducing the nucleic acid molecule into a suitable expression system that allows for the incorporation of the non-canonical amino acid Z into the polypeptide;
c. expressing the nucleic acid molecule in said expression system; and
d. allowing the functional group FG₁, and whenever present, FG₂, to react with the side-chain sulfhydryl group (—SH) of the cysteine (Cys) residue(s), thereby producing a thioether-linked cyclic peptide fused to the immunoglobulin Fc region or fragment thereof.

In one embodiment, Z may be of the amino acid structure:

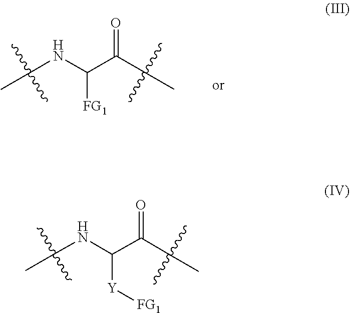

wherein FG₁ is a functional group selected from the group consisting of —(CH₂)ₙX, where X is F, Cl, Br, or I and n is an integer number from 1 to 10; —C(O)CH₂X, where X is F, Cl, Br, or I; —CH(R′)X, where X is F, Cl, Br, or I; —C(O)CH(R′)X, where X is F, Cl, Br, or I; —OCH₂CH₂X, where X is F, Cl, Br, or I; —C(O)CH═C═C(R′)(R″); —SO₂C(R′)═C(R′)(R″); —C(O)C(R′)═C(R′)(R″); —C(R′)═C(R′)C(O)OR′; —C(R′)═C(R′)C(O)N(R′)(R″); —C(R′)═C(R′)—CN; —C(R′)═C(R′)—NO₂; —C≡C—C(O)OR′; —C≡C—C(O)N(R′)(R″); unsubstituted or substituted oxirane, unsubstituted or substituted aziridine; 1,2-oxathiolane 2,2-dioxide; 4-fluoro-1,2-oxathiolane 2,2-dioxide; and 4,4-difluoro-1,2-oxathiolane 2,2-dioxide; where each R′ and R″ is independently H, an aliphatic, a substituted aliphatic, an aryl, or a substituted aryl group; and wherein Y is a linker group selected from the group consisting of aliphatic, aryl, substituted aliphatic, substituted aryl, heteroatom-containing aliphatic, heteroatom-containing aryl, substituted heteroatom-containing aliphatic, substituted heteroatom-containing aryl, alkoxy, and aryloxy groups.

Additional embodiments include where Z is a non-canonical amino acid carrying a side-chain functional group FG₁, where FG₁ is a functional group —(CH₂)ₙX, where X is F, Cl, Br, or I and n is an integer number from 1 to 10. Other embodiments include where Z is a non-canonical amino acid carrying a side-chain functional group FG₁, where FG₁ is a functional group —C(O)CH₂X, where X is F, Cl, Br, or I. Other embodiments include where Z is a non-canonical amino acid carrying a side-chain functional group FG₁, where FG₁ is a functional group —CH(R′)X, where X is F, Cl, Br, or I. Other embodiments include where Z is a non-canonical amino acid carrying a side-chain functional group FG₁, where FG₁ is a functional group —C(O)CH(R′)X, where X is F, Cl, Br, or I. Other embodiments include where Z is a non-canonical amino acid carrying a side-chain functional group FG₁, where FG₁ is a functional group —OCH₂CH₂X, where X is F, Cl, Br, or I. Other embodiments include where Z is a non-canonical amino acid carrying a side-chain functional group FG₁, where FG₁ is a functional group —C(O)CH═C═C(R′)(R″). Other embodiments include where Z is a non-canonical amino acid carrying a side-chain functional group FG₁, where FG₁ is a functional group —SO₂C(R′)═C(R′)(R″). Other embodiments include where Z is a non-canonical amino acid carrying a side-chain functional group FG₁, where FG₁ is a functional group —C(O)C(R′)═C(R′)(R″). Other embodiments include where Z is a non-canonical amino acid carrying a side-chain functional group FG₁, where FG₁ is a functional group —C(R′)═C(R′)C(O)OR′. Other embodiments include where Z is a non-canonical amino acid carrying a side-chain functional group FG₁, where FG₁ is a functional group —C(R′)═C(R′)C(O)N(R′)(R″). Other embodiments include where Z is a non-canonical amino acid carrying a side-chain functional group FG₁, where FG₁ is a functional group —C(R′)═C(R′)—CN. Other embodiments include where Z is a non-canonical amino acid carrying a side-chain functional group FG₁, where FG₁ is a functional group —C(R′)═C(R′)—NO₂. Other embodiments include where Z is a non-canonical amino acid carrying a side-chain functional group FG₁, where FG₁ is a functional group —C≡C—C(O)OR′. Other embodiments include where Z is a non-canonical amino acid carrying a side-chain functional group FG₁, where FG₁ is a functional group —C≡C—C(O)N(R′)(R″). Other embodiments include where Z is a non-canonical amino acid carrying a side-chain functional group FG₁, where FG₁ is a functional group selected from the group consisting of unsubstituted or substituted oxirane, unsubstituted or substituted aziridine, 1,2-oxathiolane 2,2-dioxide, 4-fluoro-1,2-oxathiolane 2,2-dioxide, and 4,4-difluoro-1,2-oxathiolane 2,2-dioxide. Each R′ and R″ is independently H, an aliphatic, a substituted aliphatic, an aryl, or a substituted aryl group.

Preferably, Z is an amino acid of structure (IV) and Y is a linker group selected from the group consisting of C₁-C₂₄ alkyl, C₁-C₂₄ substituted alkyl, C₁-C₂₄ substituted heteroatom-containing alkyl, C₁-C₂₄ substituted heteroatom-containing alkyl, C₂-C₂₄ alkenyl, C₂-C₂₄ substituted alkenyl, C₂-C₂₄ substituted heteroatom-containing alkenyl, C₂-C₂₄ substituted heteroatom-containing alkenyl, C₅-C₂₄ aryl, C₅-C₂₄ substituted aryl, C₅-C₂₄ substituted heteroatom-containing aryl, C₅-C₂₄ substituted heteroatom-containing aryl, C₁-C₂₄ alkoxy, and C₅-C₂₄ aryloxy groups. Also preferably, is a linker group selected from the group consisting of —CH₂—C₆H₄—, —CH₂—C₆H₄—O—, —CH₂—C₆H₄—NH—, —(CH₂)₄—, —(CH₂)₄NH—, (CH₂)₄NHC(O)—, and —(CH₂)₄NHC(O)O—.

Preferably, the amino acid Z may be selected from the group consisting of 4-(chloromethyl)-phenylalanine, 3-(chloromethyl)-phenylalanine, 4-(2-bromoethoxy)-phenylalanine, 3-(2-bromoethoxy)-phenylalanine, 4-(2-chloroethoxy)-phenylalanine, 4-(4-bromobutoxy)-phenylalanine, 4-(4-chlorobutoxy)-phenylalanine, 3-(4-bromobutoxy)-phenylalanine, 3-(4-bromobutoxy)-phenylalanine, 3-(2-chloroethoxy)-phenylalanine, 4-(1-bromoethyl)-phenylalanine, 3-(1-bromoethyl)-phenylalanine, 4-(aziridin-1-yl)-phenylalanine, 3-(aziridin-1-yl)-phenylalanine, 4-acrylamido-phenylalanine, 3-acrylamido-phenylalanine, 4-(2-fluoro-acetamido)-phenylalanine, 3-(2-fluoro-acetamido)-phenylalanine, 4-(2-chloro-acetamido)-phenylalanine, 3-(2-chloro-acetamido)-phenylalanine, 4-(2-bromo-acetamido)-phenylalanine, 3-(2-bromo-acetamido)-phenylalanine, 4-(acrylamido)-phenylalanine, 3-(acrylamido)-phenylalanine, 4-(vinylsulfonamido)-phenylalanine, 3-(vinylsulfonamido)-phenylalanine, 3-(2-fluoro-acetyl)-phenylalanine, 4-(2-fluoro-acetyl)-phenylalanine, N^{ε}-((2-bromoethoxy)carbonyl)-lysine, N^{ε}-((2-chloroethoxy)carbonyl)-lysine, N^{ε}-(buta-2,3-dienoyl)-lysine, N^{ε}-acryl-lysine, N^{ε}-crotonyl-lysine, N^{ε}-(2-fluoro-acetyl)-lysine, N^{ε}-(2-chloro-acetyl)-lysine, N^{ε}-(2-bromoacetyl)-lysine, and N^{ε}-vinylsulfonyl-lysine.

In some embodiments, a method is provided for making an artificial polypeptide, the method comprising:
a. providing a nucleic acid molecule encoding for a polypeptide of structure:

(AA)ₘ-Cys-(AA)ₙ-Z2-(AA)ₒ-Cys-(AA)ₚ (V)
wherein:
i. (AA)ₘ is an N-terminal amino acid or peptide sequence,
ii. Z2 is a non-canonical amino acid carrying two side-chain functional groups FG₁ and FG₂, these FG₁ and FG₂ being a functional group independently selected from the group consisting of —(CH₂)ₙX, where X is F, Cl, Br, or I and n is an integer number from 1 to 10; —C(O)CH₂X, where X is F, Cl, Br, or I; —CH(R′)X, where X is F, Cl, Br, or I; —C(O)CH(R′)X, where X is F, Cl, Br, or I; —OCH₂CH₂X, where X is F, Cl, Br, or I; —C(O)CH═C═C(R′)(R″); —SO₂C(R′)═C(R′)(R″); —C(O)C(R′)═C(R′)(R″); —C(R′)═C(R′)C(O)OR′; C(R′)═C(R′)C(O)N(R′)(R″); —C(R′)═C(R′)—CN; —C(R′)═C(R′)—NO₂; —C≡C—C(O)OR′; —C≡C—C(O)N(R′)(R″); unsubstituted or substituted oxirane; unsubstituted or substituted aziridine; 1,2-oxathiolane 2,2-dioxide; 4-fluoro-1,2-oxathiolane 2,2-dioxide; and 4,4-difluoro-1,2-oxathiolane 2,2-dioxide, where each R′ and R″ is independently H, an aliphatic, a substituted aliphatic, an aryl, or a substituted aryl group,
iii. (AA)ₙ is a target peptide sequence,
iv. (AA)ₒ is a second target peptide sequence,
v. (AA)ₚ is a C-terminal amino acid or peptide sequence,
vii. at least one of (AA)ₚ and (AA)ₘ comprises the Fc region of an immunoglobulin molecule or fragment thereof;
b. introducing the nucleic acid molecule into a suitable expression system that allows for the incorporation of the non-canonical amino acid Z2 into the polypeptide;
c. expressing the nucleic acid molecule in said expression system; and
d. allowing the functional group FG₁, and whenever present, FG₂, to react with the side-chain sulfhydryl group (—SH) of the cysteine (Cys) residue(s), thereby producing a thioether-linked cyclic peptide fused to the immunoglobulin Fc region or fragment thereof.

According to the method, (AA)ₘ is a N-terminal sequence comprising at least one amino acid, where AA corresponds to a generic amino acid residue and m corresponds to the number of amino acid residues composing such sequence. (AA)ₘ is also referred to as “N-terminal tail”. (AA)ₚ is a C-terminal sequence that has 0 or at least one amino acid, where AA corresponds to a generic amino acid residue and p corresponds to the number of amino acid residues composing such sequence. (AA)ₚ is also referred to as “C-terminal tail”. (AA)ₙ (and (AA)ₒ, when present) is a peptide sequence of variable length (also referred to as “target peptide sequence”), where AA corresponds to a generic amino acid residue and n (and o, when present) corresponds to the number of amino acid residues composing such peptide sequence(s). Cys is a cysteine amino acid residue. Z is an amino acid that carries a side-chain functional group FG₁, which can react with the side-chain sulfhydryl group (—SH) of the cysteine residue to form a stable thioether bond.

In certain embodiments, Z2 is a non-canonical amino acid carrying two side-chain functional groups, including FG₁ which is a functional group —(CH₂)ₙX, where X is F, Cl, Br, or I and n is an integer number from 1 to 10. In certain embodiments, Z2 is a non-canonical amino acid carrying two side-chain functional groups, including FG₂ which is a functional group —(CH₂)ₙX, where X is F, Cl, Br, or I and n is an integer number from 1 to 10. In certain embodiments, Z2 is a non-canonical amino acid carrying two side-chain functional groups, including FG₁ which is a functional group —C(O)CH₂X, where X is F, Cl, Br, or I. In certain embodiments, Z2 is a non-canonical amino acid carrying two side-chain functional groups, including FG₂ which is a functional group —C(O)CH₂X, where X is F, Cl, Br, or I. In certain embodiments, Z2 is a non-canonical amino acid carrying two side-chain functional groups, including FG₁ which is a functional group —CH(R′)X, where X is F, Cl, Br, or I. In certain embodiments, Z2 is a non-canonical amino acid carrying two side-chain functional groups, including FG₂ which is a functional group —CH(R′)X, where X is F, Cl, Br, or I. In certain embodiments, Z2 is a non-canonical amino acid carrying two side-chain functional groups, including FG₁ which is a functional group —C(O)CH(R′)X, where X is F, Cl, Br, or I. In certain embodiments, Z2 is a non-canonical amino acid carrying two side-chain functional groups, including FG₂ which is a functional group —C(O)CH(R′)X, where X is F, Cl, Br, or I. In certain embodiments, Z2 is a non-canonical amino acid carrying two side-chain functional groups, including FG₁ which is a functional group —OCH₂CH₂X, where X is F, Cl, Br, or I. In certain embodiments, Z2 is a non-canonical amino acid carrying two side-chain functional groups, including FG₂ which is a functional group —OCH₂CH₂X, where X is F, Cl, Br, or I. In certain embodiments, Z2 is a non-canonical amino acid carrying two side-chain functional groups, including FG₁ which is a functional group —C(O)CH═C═C(R′)(R″). In certain embodiments, Z2 is a non-canonical amino acid carrying two side-chain functional groups, including FG₂ which is a functional group —C(O)CH═C═C(R′)(R″). In certain embodiments, Z2 is a non-canonical amino acid carrying two side-chain functional groups, including FG₁ which is a functional group —SO₂C(R′)═C(R′)(R″). In certain embodiments, Z2 is a non-canonical amino acid carrying two side-chain functional groups, including FG₂ which is a functional group —SO₂C(R′)═C(R′)(R″). In certain embodiments, Z2 is a non-canonical amino acid carrying two side-chain functional groups, including FG; which is a functional group —C(O)C(R′)═C(R′)(R″). In certain embodiments, Z2 is a non-canonical amino acid carrying two side-chain functional groups, including FG₂ which is is a functional group —C(O)C(R′)═C(R′)(R″). In certain embodiments, Z2 is a non-canonical amino acid carrying two side-chain functional groups, including FG₁ which is a functional group —C(R′)═C(R′)C(O)OR′. In certain embodiments, Z2 is a non-canonical amino acid carrying two side-chain functional groups, including FG₂ which is a functional group —C(R′)═C(R′)C(O)N(R′)(R″). In certain embodiments, Z2 is a non-canonical amino acid carrying two side-chain functional groups, including FG₁ which is a functional group —C(R′)═C(R′)—CN. In certain embodiments, Z2 is a non-canonical amino acid carrying two side-chain functional groups, including FG₂ which is a functional group —C(R′)═C(R′)—CN. In certain embodiments, Z2 is a non-canonical amino acid carrying two side-chain functional groups, including FG₁ which is a functional group —C(R′)═C(R′)—NO₂. In certain embodiments, Z2 is a non-canonical amino acid carrying two side-chain functional groups, including FG₂ which is a functional group —C(R′)═C(R′)—NO₂. In certain embodiments, Z2 is a non-canonical amino acid carrying two side-chain functional groups, including FG₁ which is a functional group —C≡C—C(O)OR′. In certain embodiments, Z2 is a non-canonical amino acid carrying two side-chain functional groups, including FG₂ which is a functional group —C≡C—C(O)OR′. In certain embodiments, Z2 is a non-canonical amino acid carrying two side-chain functional groups, including FG₁ which is a functional group —C≡C—C(O)N(R′)(R″). In certain embodiments, Z2 is a non-canonical amino acid carrying two side-chain functional groups, including FG₂ which is a functional group —C≡C—C(O)N(R′)(R″). In certain embodiments, Z2 is a non-canonical amino acid carrying two side-chain functional groups, including FG₁ which is a functional group selected from the group consisting of unsubstituted or substituted oxirane, unsubstituted or substituted aziridine, 1,2-oxathiolane 2,2-dioxide, 4-fluoro-1,2-oxathiolane 2,2-dioxide, and 4,4-difluoro-1,2-oxathiolane 2,2-dioxide. In certain embodiments, Z2 is a non-canonical amino acid carrying two side-chain functional groups, including FG₂ which is a functional group selected from the group consisting of unsubstituted or substituted oxirane, unsubstituted or substituted aziridine, 1,2-oxathiolane 2,2-dioxide, 4-fluoro-1,2-oxathiolane 2,2-dioxide, and 4,4-difluoro-1,2-oxathiolane 2,2-dioxide. Each R′ and R″ is independently H, an aliphatic, a substituted aliphatic, an aryl, or a substituted aryl group.

As disclosed herein, the ability of an artificial polypeptide of formula (I) or (II) (also referred herein to as “precursor polypeptide”) to produce a cyclopeptide-Fc region fusion is conferred by the ability of the nucleophilic sulfhydryl group carried by the cysteine residue to react intramolecularly with the electrophilic functional group FG₁ carried by the amino acid Z, thereby forming a covalent, inter-side-chain thioether bond. Depending on the nature of FG₁, this reaction proceeds via a thiol-mediated nucleophilic substitution reaction, a thiol-mediated Michael-type addition reaction, or a radical thiol-ene or thiol-yne reaction. Whereas the electrophilic functional group FG₁ in the precursor polypeptide could in principle react intermolecularly with free cysteine or other thiol-containing molecules contained in the expression system (e.g., glutathione), it was discovered by the inventors that appropriate functional groups FG₁ can be found so that the desired intramolecular thioether-bond forming reaction occurs exclusively or preferentially over the undesired intermolecular side-reactions. This result can be achieved because of the spatial proximity between the nucleophilic cysteine residue and the electrophilic Z amino acid, resulting in an increased effective concentration of the reacting species (i.e. —SH and FG₁ groups, respectively) in the intramolecular settings as compared to the intermolecular settings, which in turn favors the intramolecular peptide cyclization reaction over undesired intermolecular reactions.

FIG. 1 shows a schematic representation of a general method for making a cyclopeptibody molecule (1) from precursor polypeptides (2) of general formula (I) (AA)ₘ-Z-(AA)ₙ-Cys-(AA)ₚ. There is a polynucleotide (3) that encodes the precursor polypeptide, which includes an N-terminal tail (AA)ₘ (3), an electrophilic amino acid Z (4), the target peptide sequence (AA)ₙ (6), a cysteine (7) and a C-terminal tail (AA)ₚ (8). An Fc domain is comprised within the N-terminal tail (AA)ₘ (3), or within the C-terminal tail (AA)ₚ (8), or both. W corresponds to the linker group resulting from the bond-forming reaction between the functional group FG₁ and the —S of the cysteine residue (C).

FIG. 2 shows a schematic representation of a general method for making a cyclopeptibody molecule (1) from precursor polypeptides (2) of general formula (II) (AA)ₘ-Cys-(AA)ₙ-Z-(AA)ₚ. There is a polynucleotide (3) that encodes the precursor polypeptide, which includes an N-terminal tail (AA)ₘ (3), a cysteine (7), the target peptide sequence (AA) n (6), an electrophilic amino acid Z (4) and a C-terminal tail (AA)ₚ (8). An Fc domain is comprised within the N-terminal tail (AA)ₘ (3), or within the C-terminal tail (AA)ₚ (8), or both. W corresponds to the linker group resulting from the bond-forming reaction between the functional group FG₁ and the cysteine residue.

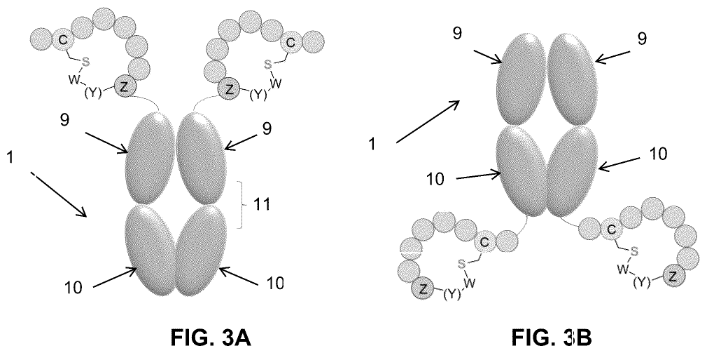

FIG. 3 is a schematic representation of a cyclopeptibody (1) comprising a cyclic peptide fused to the N-terminus of an Fc domain (FIG. 3A) or fused to the C-terminus of an Fc domain (FIG. 3B), as prepared according to the methods of the invention. W corresponds to the linker group resulting from the bond-forming reaction between the functional group FG₁ and the cysteine residue. An electrophilic amino acid Z including a linker group Y is also present. In some embodiments, the immunoglobulin Fc region in the cyclopeptibody molecule (1) is a polypeptide that comprises the heavy chain constant region 2 (9), heavy chain constant region 3 (10) and hinge region (11).

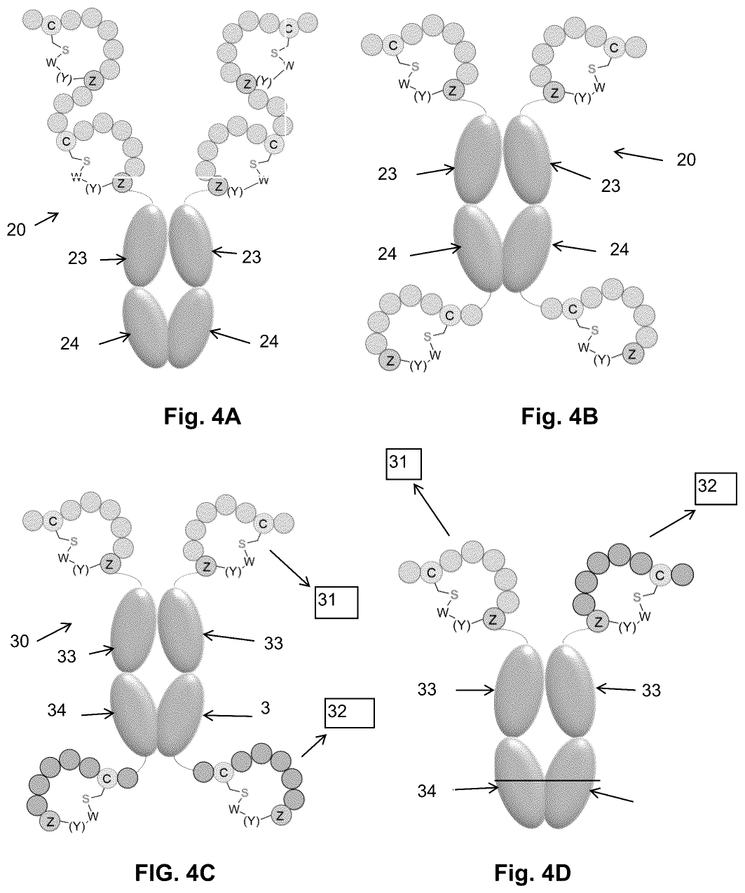

FIG. 4 is a schematic representation of a polyvalent cyclopeptibody molecule (20) (FIG. 4A-4B) and bispecific cyclopeptibody molecule (30) (FIG. 4C-4D), as prepared according to the methods of the invention. There are two target proteins (21 and 22 or 31 and 32). W corresponds to the linker group resulting from the bond-forming reaction between the functional group FG₁ and the cysteine residue. An electrophilic amino acid Z including a linker group Y is also present. In some embodiments, the immunoglobulin Fc region in the cyclopeptibody molecule (20 or 30) is a polypeptide that comprises the heavy chain constant region 2 (23 or 33), heavy chain constant region 3 (24 or 34).

Similar considerations can be made in the context of certain embodiments, wherein a precursor polypeptide of formula (V) along with a bifunctional cysteine-reactive amino acid capable of forming thioether bonds with two cysteine residues within the polypeptide (residue Z2) is used.

A first advantage of the methods described herein is that they provide a convenient and highly versatile approach for enabling the fusion of a biologically active cyclic peptide to the Fc region of an immunoglobulin. Accordingly, these methods allow for the generation of a fusion protein that combines the recognition properties and advantageous features of a cyclic peptide (e.g., target affinity/specificity) with the extended in vivo half-life and/or immunomodulatory properties mediated by the immunoglobulin Fc region.

Compared to the cyclic peptide alone, the cyclopeptide-Fc fusion proteins disclosed here can provide other distinct advantages such as enhanced target affinity due to bivalency and thus avidity effects, as illustrated in Examples 5-10. In addition, compared to the cyclic peptide alone, genetic fusion of a bioactive cyclic peptide to an immunoglobulin Fc domain can confer said bioactive cyclic peptide with extended in vivo half-life, which is desirable for therapeutic applications. Furthermore, fusion of a bioactive cyclopeptide to an immunoglobulin Fc domain can enable detection of a target biomolecule recognized by the cyclopeptide (e.g., cell receptor) using commonly available Fc-binding reagents (e.g., anti-Fc antibodies and fluorescently labeled derivatives thereof). Moreover, if desired, fusion of a bioactive cyclopeptide to an immunoglobulin Fc domain can enable targeting of an Fc-mediated immune response (e.g., complement-dependent cytotoxicity (CDC) or antibody-dependent cellular cytotoxicity (ADCC)) against an antigen, pathogenic agent (e.g., pathogenic virus or bacteria), or cell (e.g., cancer cell) recognized by the cyclic peptide.

A third advantage of the methods disclosed herein is that they enable the production of Fc-fused peptides whose conformational flexibility is restrained by virtue of one or more intramolecular thioether linkage(s). As illustrated in Example 8, this feature can confer these molecules with advantageous properties such as, for example, enhanced binding affinity, target specificity, and/or increased stability against proteolysis, compared to linear peptides or peptides lacking the intramolecular thioether linkage. In addition, unlike disulfide bridges, the thioether linkage in the cyclopeptide-Fc fusions disclosed herein is stable and chemical inert toward physiological reducing agents such as, for example, glutathione and/or free cysteines, chemical reductants commonly used for antibody bioconjugation (e.g., TCEP and other phosphines), and it is stable in reducing environments, such as the intracellular milieu.

A fourth advantage of the methods disclosed herein is that the cyclic peptide genetically fused to the immunoglobulin Fc region can be prepared in many different topologies, e.g., through variation of the length and composition of the target peptide sequence ((AA) n), variation of the structure of the amino acid Z, variation of the position of the amino acid Z relative to the cysteine residue (e.g., precursor polypeptide (I) versus (II)), variation of the length and composition of the N-terminal tail ((AA)ₘ), and variation of the length and composition of the C-terminal tail ((AA)ₚ). Further structural diversification can be achieved by combining multiple Z/Cys pairs within the same precursor polypeptide or by using bifunctional cysteine-reactive amino acids (Z2) in order to obtain polycyclic and bicyclic peptides. Accordingly, and because of the genetically encoded and ribosomal nature of the precursor polypeptides and spontaneous (i.e., not chemically or enzymatically) formation of the thioether bridge, the methods and compositions described herein can be used to produce a wide variety of structurally and functionally diverse macrocyclic peptides fused to the Fc region of an immunoglobulin molecule. Because of the wide range of cyclopeptide structures accessible with the methods disclosed herein, these cyclopeptibody molecules can be constructed and applied for binding, detection, imaging, and/or causing inhibition (or activation) of a broad range of target biomolecules. As illustrated in Examples 5-10, cyclopeptibodies capable of binding, detecting, or inhibiting a diverse set of target proteins with high activity and selectivity could be prepared using the methods disclosed herein.

A fifth advantage of the methods disclosed herein is that the cyclopeptide can be fused to the N-terminus or the C-terminus of an immunoglobulin Fc region or fragment thereof. Depending on the application, this capability is beneficial to optimize the function of the cyclic peptide (e.g., protein binding affinity) and/or of the Fc domain (e.g., Fc receptor recognition), or both.

A sixth advantage of the methods disclosed herein is that they can also enable the preparation of cyclopeptibody molecules that contain multiple copies of the same cyclic peptide or, alternatively, cyclic peptides with different target specificity. In turn, this capability can be beneficial to obtain cyclopeptibody molecules featuring enhanced target affinity/specificity (e.g., due to avidity effects deriving from polyvalency) and dual target specificity, respectively. Polyvalent and bi-specific cyclopeptibodies can be useful to induce the homo- and heterodimerization of receptors, respectively, or to inhibit or promote the interaction between two target molecules for a variety of applications, including therapeutic applications.

A seventh advantage of the methods described herein is that they allow for the production of cyclopeptibody molecules in any cell-based expression host, including bacterial, yeast, insect, or mammalian cells, or a cell-derived expression system such as a cell lysate. Adding to this convenience, the cyclopeptide-Fc fusions can be directly isolated from these expression hosts without the need of chemical modification or other manipulation that could impart the function of these proteins. Furthermore, unlike full-length antibodies, the cyclopeptibodies of the invention can be produced and isolated from bacterial cells (e.g., E. coli), as illustrated in Example 2.

Because of these advantageous features, the methods described herein can be useful to prepare and isolate cyclopeptibodies for binding, detection, imaging, and inhibition or activation of a broad range of target biomolecules in the context of a variety of applications, including therapeutic applications.

In some embodiments, Z is an amino acid of structure:

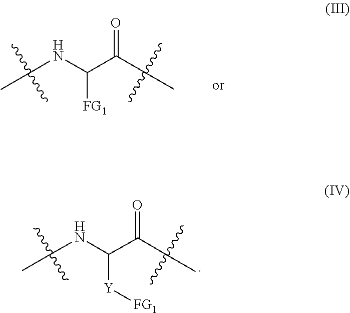

wherein FG₁ is a functional group selected from the group consisting of —(CH₂)ₙX, where X is F, Cl, Br, or I and n is an integer number from 1 to 10; —C(O)CH₂X, where X is F, Cl, Br, or I; —CH(R′)X, where X is F, Cl, Br, or I; —C(O)CH(R′)X, where X is F, Cl, Br, or I; —OCH₂CH₂X, where X is F, Cl, Br, or I; —C(O)CH═C═C(R′)(R″); —SO₂C(R′)═C(R′)(R″); —C(O)C(R′)═C(R′)(R″); —C(R′)═C(R′)C(O)OR′; —C(R′)═C(R′)C(O)N(R′)(R″); —C(R′)═C(R′)—CN; —C(R′)═C(R′)—NO₂; —C≡C—C(O)OR′; —C≡C—C(O)N(R′)(R″); unsubstituted or substituted oxirane; unsubstituted or substituted aziridine; 1,2-oxathiolane 2,2-dioxide; 4-fluoro-1,2-oxathiolane 2,2-dioxide; and 4,4-difluoro-1,2-oxathiolane 2,2-dioxide, where each R′ and R″ is independently H, an aliphatic, a substituted aliphatic, an aryl, or a substituted aryl group; and
wherein Y is a linker group selected from the group consisting of aliphatic, aryl, substituted aliphatic, substituted aryl, heteroatom-containing aliphatic, heteroatom-containing aryl, substituted heteroatom-containing aliphatic, substituted heteroatom-containing aryl, alkoxy, and aryloxy groups.

In some embodiments, Z is an amino acid of structure:

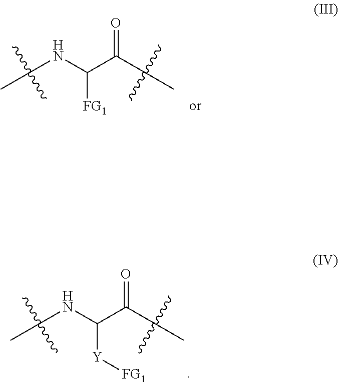

wherein FG₁ is a functional group —(CH₂)ₙX, where X is F, Cl, Br, or I and n is an integer number from 1 to 10. In some embodiments, FG₁ is a functional group —C(O)CH₂X, where X is F, Cl, Br, or I. In some embodiments, FG₁ is a functional group —CH(R′)X, where X is F, Cl, Br, or I. In some embodiments, FG₁ is a functional group —C(O)CH(R′)X, where X is F, Cl, Br, or I. In some embodiments, FG₁ is a functional group —OCH₂CH₂X, where X is F, Cl, Br, or I. In some embodiments, FG₁ is a functional group —C(O)CH═C═C(R′)(R″). In some embodiments, FG₁ is a functional group —SO₂C(R′)═C(R′)(R″). In some embodiments, FG₁ is a functional group —C(O)C(R′)═C(R′)(R″). In some embodiments, FG₁ is a functional group —C(R′)═C(R′)C(O)OR′. In some embodiments, FG₁ is a functional group —C(R′)═C(R′)C(O)N(R′)(R″). In some embodiments, FG₁ is a functional group —C(R′)═C(R′)—CN. In some embodiments, FG₁ is a functional group —C(R′)═C(R′)—NO₂. In some embodiments, FG₁ is a functional group —C≡C—C(O)OR′. In some embodiments, FG₁ is a functional group —C≡C—C(O)N(R′)(R″). In some embodiments, FG₁ is a functional group selected from the group consisting of unsubstituted or substituted oxirane, unsubstituted or substituted aziridine, 1,2-oxathiolane 2,2-dioxide, 4-fluoro-1,2-oxathiolane 2,2-dioxide, and 4,4-difluoro-1,2-oxathiolane 2,2-dioxide. Each R′ and R″ is independently H, an aliphatic, a substituted aliphatic, an aryl, or a substituted aryl group. In some embodiments, Y is a linker group selected from the group consisting of aliphatic, aryl, substituted aliphatic, substituted aryl, heteroatom-containing aliphatic, heteroatom-containing aryl, substituted heteroatom-containing aliphatic, substituted heteroatom-containing aryl, alkoxy, and aryloxy groups.

In some embodiments, Z is an amino acid of structure (IV) wherein Y is a linker group selected from the group consisting of C₁-C₂₄ alkyl, C₁-C₂₄ substituted alkyl, C₁-C₂₄ substituted heteroatom-containing alkyl, C₁-C₂₄ substituted heteroatom-containing alkyl, C₂-C₂₄ alkenyl, C₂-C₂₄ substituted alkenyl, C₂-C₂₄ substituted heteroatom-containing alkenyl, C₂-C₂₄ substituted heteroatom-containing alkenyl, C₅-C₂₄ aryl, C₅-C₂₄ substituted aryl, C₅-C₂₄ substituted heteroatom-containing aryl, C₅-C₂₄ substituted heteroatom-containing aryl, C₁-C₂₄ alkoxy, C₅-C₂₄ aryloxy groups.

In some embodiments, Z is an amino acid of structure (IV) wherein Y is a linker group selected from —CH₂—C₆H₄—, —CH₂—C₆H₄—O—, —CH₂—C₆H₄—NH—, —(CH₂)₄—, —(CH₂)₄NH—, —(CH₂)₄NHC(O)—, and —(CH₂)₄NHC(O)O—.

In specific embodiments, the amino acid Z is selected from the group consisting of 4-(chloromethyl)-phenylalanine, 3-(chloromethyl)-phenylalanine, 4-(2-bromoethoxy)-phenylalanine, 4-(2-bromoethoxy)-phenylalanine, 3-(2-bromoethoxy)-phenylalanine, 4-(2-chloroethoxy)-phenylalanine, 4-(4-bromobutoxy)-phenylalanine, 4-(4-chlorobutoxy)-phenylalanine, 3-(4-bromobutoxy)-phenylalanine, 3-(4-bromobutoxy)-phenylalanine, 3-(2-chloroethoxy)-phenylalanine, 4-(1-bromoethyl)-phenylalanine, 3-(1-bromoethyl)-phenylalanine, 4-(aziridin-1-yl)-phenylalanine, 3-(aziridin-1-yl)-phenylalanine, 4-acrylamido-phenylalanine, 3-acrylamido-phenylalanine, 4-(2-fluoro-acetamido)-phenylalanine, 3-(2-fluoro-acetamido)-phenylalanine, 4-(2-chloro-acetamido)-phenylalanine, 3-(2-chloro-acetamido)-phenylalanine, 4-(2-bromo-acetamido)-phenylalanine, 3-(2-bromo-acetamido)-phenylalanine, 4-(acrylamido)-phenylalanine, 3-(acrylamido)-phenylalanine, 4-(vinylsulfonamido)-phenylalanine, 3-(vinylsulfonamido)-phenylalanine, 3-(2-fluoro-acetyl)-phenylalanine, 4-(2-fluoro-acetyl)-phenylalanine, N^{ε}-((2-bromoethoxy)carbonyl)-lysine, N^{ε}-((2-chloroethoxy)carbonyl)-lysine, N^{ε}-(buta-2,3-dienoyl)-lysine, N^{ε}-acryl-lysine, N^{ε}-crotonyl-lysine, N^{ε}-(2-fluoro-acetyl)-lysine, N^{ε}-(2-chloro-acetyl)-lysine, N^{ε}-(2-bromoacetyl)-lysine, and N^{ε}-vinylsulfonyl-lysine.

In some embodiments, Z2 is an amino acid of structure:

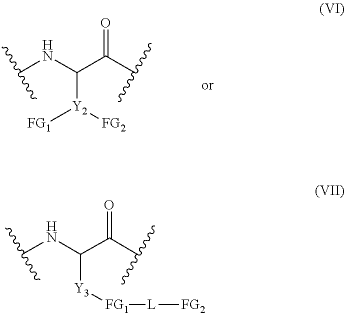

wherein FG₁ and FG₂ are a functional group independently selected from the group consisting of —(CH₂)ₙX, where X is F, Cl, Br, or I and n is an integer number from 1 to 10; —C(O)CH₂X, where X is F, Cl, Br, or I; —CH(R′)X, where X is F, Cl, Br, or I; —C(O)CH(R′)X, where X is F, Cl, Br, or I; —OCH₂CH₂X, where X is F, Cl, Br, or I; —C(O)CH═C═C(R′)(R″), —SO₂C(R′)═C(R′)(R″), —C(O)C(R′)═C(R′)(R″), —C(R′)═C(R′)C(O)OR′, C(R′)═C(R′)C(O)N(R′)(R″), —C(R′)═C(R′)—CN, —C(R′)═C(R′)—NO₂, —C≡C—C(O)OR′, —C≡C—C(O)N(R′)(R″), unsubstituted or substituted oxirane, unsubstituted or substituted aziridine, 1,2-oxathiolane 2,2-dioxide, 4-fluoro-1,2-oxathiolane 2,2-dioxide, and 4,4-difluoro-1,2-oxathiolane 2,2-dioxide, where each R′ and R″ is independently H, an aliphatic, a substituted aliphatic, an aryl, or a substituted aryl group; and wherein Y₂, Y₃, and L are linker groups selected from the group consisting of aliphatic, aryl, substituted aliphatic, substituted aryl, heteroatom-containing aliphatic, heteroatom-containing aryl, substituted heteroatom-containing aliphatic, substituted heteroatom-containing aryl, alkoxy, aryloxy groups.

In some embodiments, Z2 is an amino acid of structure:

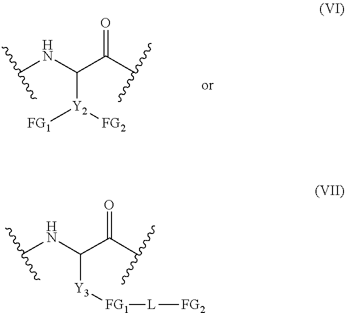

wherein FG₁ is a functional group —(CH₂)ₙX, where X is F, Cl, Br, or I and n is an integer number from 1 to 10. In other embodiments, FG₁ is a functional group —C(O)CH₂X, where X is F, Cl, Br, or I. In other embodiments, FG₁ is a functional group —CH(R′)X, where X is F, Cl, Br, or I. In other embodiments, FG₁ is a functional group —C(O)CH(R′)X, where X is F, Cl, Br, or I. In other embodiments, FG₁ is a functional group —OCH₂CH₂X, where X is F, Cl, Br, or I. In other embodiments, FG₁ is a functional group —C(O)CH═C═C(R′)(R″). In other embodiments, FG₁ is a functional group —SO₂C(R′)═C(R′)(R″). In other embodiments, FG₁ is a functional group —C(O)C(R′)═C(R′)(R″). In other embodiments, FG₁ is a functional group —C(R′)═C(R′)C(O)OR′. In other embodiments, FG₁ is a functional group —C(R′)═C(R′)C(O)N(R′)(R″). In other embodiments, FG₁ is a functional group —C(R′)═C(R′)—CN. In other embodiments, FG₁ is a functional group —C(R′)═C(R′)—NO₂. In other embodiments, FG₁ is a functional group —C≡C—C(O)OR′. In other embodiments, FG₁ is a functional group —C≡C—C(O)N(R′)(R″). In other embodiments, FG₁ is a functional group selected from the group consisting of unsubstituted or substituted oxirane, unsubstituted or substituted aziridine, 1,2-oxathiolane 2,2-dioxide, 4-fluoro-1,2-oxathiolane 2,2-dioxide, and 4,4-difluoro-1,2-oxathiolane 2,2-dioxide. In other embodiments, FG₂ is a functional group —(CH₂)ₙX, where X is F, Cl, Br, or I and n is an integer number from 1 to 10. In other embodiments, FG₂ is a functional group —C(O)CH₂X, where X is F, Cl, Br, or I. In other embodiments, FG₂ is a functional group —CH(R′)X, where X is F, Cl, Br, or I. In other embodiments, FG₂ is a functional group —C(O)CH(R′)X, where X is F, Cl, Br, or I. In other embodiments, FG₂ is a functional group —OCH₂CH₂X, where X is F, Cl, Br, or I. In other embodiments, FG₂ is a functional group —C(O)CH═C═C(R′)(R″). In other embodiments, FG₂ is a functional group —SO₂C(R′)═C(R′)(R″). In other embodiments, FG₂ is a functional group —C(O)C(R′)═C(R′)(R″). In other embodiments, FG₂ is a functional group —C(R′)═C(R′)C(O)OR′. In other embodiments, FG₂ is a functional group —C(R′)═C(R′)C(O)N(R′)(R″). In other embodiments, FG₂ is a functional group —C(R′)═C(R′)—CN. In other embodiments, FG₂ is a functional group —C(R′)═C(R′)—NO₂. In other embodiments, FG₂ is a functional group —C≡C—C(O)OR′. In other embodiments, FG₂ is a functional group —C≡C—C(O)N(R′)(R″). In other embodiments, FG₂ is a functional group selected from the group consisting of unsubstituted or substituted oxirane, unsubstituted or substituted aziridine, 1,2-oxathiolane 2,2-dioxide, 4-fluoro-1,2-oxathiolane 2,2-dioxide, and 4,4-difluoro-1,2-oxathiolane 2,2-dioxide. Each R′ and R″ is independently H, an aliphatic, a substituted aliphatic, an aryl, or a substituted aryl group.

In some embodiments, Y₂, is a linker group selected from the group consisting of aliphatic, aryl, substituted aliphatic, substituted aryl, heteroatom-containing aliphatic, heteroatom-containing aryl, substituted heteroatom-containing aliphatic, substituted heteroatom-containing aryl, alkoxy, aryloxy groups. In other embodiments, Y₃ is a linker group selected from the group consisting of aliphatic, aryl, substituted aliphatic, substituted aryl, heteroatom-containing aliphatic, heteroatom-containing aryl, substituted heteroatom-containing aliphatic, substituted heteroatom-containing aryl, alkoxy, aryloxy groups. In other embodiments, L is a linker group selected from the group consisting of aliphatic, aryl, substituted aliphatic, substituted aryl, heteroatom-containing aliphatic, heteroatom-containing aryl, substituted heteroatom-containing aliphatic, substituted heteroatom-containing aryl, alkoxy, aryloxy groups.

In some embodiments, Z2 is an amino acid of structure (VI) wherein Y₂ is a linker group selected from the group consisting of C₁-C₂₄ alkyl, C₁-C₂₄ substituted alkyl, C₁-C₂₄ substituted heteroatom-containing alkyl, C₁-C₂₄ substituted heteroatom-containing alkyl, C₂-C₂₄ alkenyl, C₂-C₂₄ substituted alkenyl, C₂-C₂₄ substituted heteroatom-containing alkenyl, C₂-C₂₄ substituted heteroatom-containing alkenyl, C₅-C₂₄ aryl, C₅-C₂₄ substituted aryl, C₅-C₂₄ substituted heteroatom-containing aryl, C₅-C₂₄ substituted heteroatom-containing aryl, C₁-C₂₄ alkoxy, C₅-C₂₄ aryloxy groups.

In some embodiments, Z2 is an amino acid of structure (VI) wherein Y₂ is a linker group selected from the group consisting of —CH₂—C₆H₄—, —CH₂—C₆H₄—O—, —CH₂—C₆H₄—NH—, —CH₂—C₆H₄—OCH₂—, —(CH₂)₄NH—, —(CH₂)₄NHC(O)—, —(CH₂)₄NHC(O)O—, —(CH₂)₄NHC(O)OCH₂—,

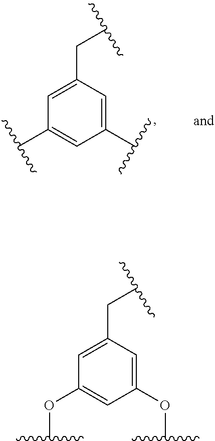 [SMILES: *Cc1cc(*)cc(*)c1 ; *Cc1cc(O*)cc(O*)c1]

In specific embodiments, the amino acid Z2 is selected from the group consisting of 3,5-bis(chloromethyl)-phenylalanine, 3,5-bis(2-bromoethoxy)-phenylalanine, 3,5-bis(2-chloroethoxy)-phenylalanine, 3,5-bis(4-bromobutoxy)-phenylalanine, 3,5-bis(4-chlorobutoxy)-phenylalanine, 3,5-bis(1-bromoethyl)-phenylalanine, 3,5-bis(4-acrylamido)-phenylalanine, 3,5-bis(2-chloro-acetamido)-phenylalanine, 3,5-bis(2-bromo-acetamido)-phenylalanine, 3,5-bis(vinylsulfonamido)-phenylalanine, 3,5-bis(aziridin-1-yl)-phenylalanine, 3,5-bis-acrylamido-phenylalanine, 3,5-bis(2-fluoro-acetamido)-phenylalanine, 3,5-bis(2-fluoro-acetyl)-phenylalanine, 4-((1,3-dibromopropan-2-yl)oxy)-phenylalanine, 4-((1,3-dichloropropan-2-yl)oxy)-phenylalanine, N^{ε}-(((1,3-dibromopropan-2-yl)oxy)carbonyl)-lysine, N^{ε}-(((1,3-dichloropropan-2-yl)oxy)carbonyl)-lysine, 4-(2,3-dibromopropoxy)-phenylalanine, 3-(2,3-dibromopropoxy)-phenylalanine, 4-(2,3-dichloropropoxy)-phenylalanine, 3-(2,3-dichloropropoxy)-phenylalanine, N^{ε}-((2,3-dibromopropoxy)carbonyl)-lysine, N^{ε}-((2,3-dichloropropoxy)carbonyl)-lysine, N^{ε}-bis-(acryl)-lysine, N^{ε}-bis-(crotonyl)-lysine, N^{ε}-bis-(2-fluoro-acetyl)-lysine, N^{ε}-bis-(2-chloro-acetyl)-lysine, N^{ε}-bis-(2-bromoacetyl)-lysine, N^{ε}-bis-(vinylsulfonyl)-lysine, 4-(2,2-dichloro-acetamido)-phenylalanine, 4-(2,2-difluoro-acetamido)-phenylalanine, 3-(2,2-dichloro-acetamido)-phenylalanine, 3-(2,2-difluoro-acetamido)-phenylalanine, 4-(2,2-dichloroacetyl)-phenylalanine, 4 and -(2,2-difluoroacetyl)-phenylalanine, 3-(2,2-dichloroacetyl)-phenylalanine, 3-(2,2-difluoroacetyl)-phenylalanine.

Artificial nucleic acid molecules for use according to the methods provided herein include, but are not limited to, those that encode for a polypeptide of general formula (I), (II), or (V) as defined above. The codon encoding for the amino acid Z (or Z2) in these polypeptides can be one of the 61 sense codons of the standard genetic code, a stop codon (TAG, TAA, TGA), or a four-base frameshift codon (e.g., TAGA, AGGT, CGGG, GGGT, CTCT). In some embodiments, the codon encoding for the amino acid Z (or Z2) within the nucleotide sequence encoding for the precursor polypeptide of formula (I), (II) or (V) is an amber stop codon (TAG), an ochre stop codon (TAA), an opal stop codon (TGA), or a four-base frameshift codon (see Example 2). In other embodiments, the codon encoding for Z (or Z2) in the nucleotide sequence encoding for these precursor polypeptides is the amber stop codon, TAG, or the 4-base codon, TAGA.

The non-canonical amino acid Z (or Z2) can be introduced into the precursor polypeptide through direct incorporation during ribosomal synthesis of the precursor polypeptide or generated post-translationally through enzymatic or chemical modification of the precursor polypeptide, or by a combination of these procedures. In some embodiments, the amino acid Z (or Z2) is introduced into the precursor polypeptide during ribosomal synthesis of the precursor polypeptide via either stop codon suppression or four-base frameshift codon suppression. In other embodiments, the amino acid Z (or Z2) is introduced into the precursor polypeptide during ribosomal synthesis of the precursor polypeptide via amber (TAG) stop codon suppression or via 4-base TAGA codon suppression.

Several methods are known in the art for introducing a non-canonical amino acid into a recombinant or in vitro translated artificial polypeptide, any of which can be applied for preparing artificial precursor polypeptides suitable for the methods disclosed herein. These art-known methods include, but are not limited to, methods for suppression of a stop codon or of a four-based frameshift codon with a non-canonical amino acid using engineered (i.e., non-naturally occurring, artificial or synthetic) tRNA/aminoacyl-tRNA synthetase (AARS) pairs (Wu and Schultz 2009; Wang, Xie, and Schultz 2006; Liu and Schultz 2010; Fekner and Chan 2011; Lang and Chin 2014). Examples of tRNA/aminoacyl-tRNA synthetase (AARS) pairs used for this purpose include, but are not limited to, engineered variants of Methanococcus jannaschii AARS/tRNA pairs (e.g., TyrRS/tRNA^{Tyr}), of Saccharomyces cerevisiae AARS/tRNA pairs (e.g., AspRS/tRNA^{Asp}, GlnRS/tRNA^{Gln}, TyrRS/tRNA^{Tyr}, and PheRS/tRNA^{Phe}), of Escherichia coli AARS/tRNA pairs (e.g., TyrRS/tRNA^{Tyr}, LeuRS/tRNA^{Leu}), of Methanosarcina mazei AARS/tRNA pairs (PyIRS/tRNA^{Pyl}), and of Methanosarcina mazei AARS/tRNA pairs (PylRS/tRNA^{Pyl}) (Wu and Schultz 2009; Wang, Xie, and Schultz 2006; Liu and Schultz 2010; Fekner and Chan 2011; Lang and Chin 2014). Alternatively, natural or engineered four-codon suppressor tRNAs and their cognate aminoacyl-tRNA synthetases can be used for the same purpose (Rodriguez, Lester, and Dougherty 2006; Neumann et al. 2010; Neumann, Slusarczyk, and Chin 2010; Anderson et al. 2004). Alternatively, a non-canonical amino acid can be incorporated into a polypeptide using chemically (Dedkova et al. 2003) or enzymatically (Bessho, Hodgson, and Suga 2002; Hartman, Josephson, and Szostak 2006) aminoacylated tRNA molecules and using a cell-free protein expression system in the presence of the aminoacylated tRNA molecules (Murakami et al. 2006; Kourouklis, Murakami, and Suga 2005). Alternatively, a non-canonical amino acid can be incorporated into a polypeptide by exploiting the promiscuity of wild-type aminoacyl-tRNA synthetase enzymes using a cell-free protein expression system, in which one or more natural amino acids are replaced with structural analogs (Hartman et al. 2007; Josephson, Hartman, and Szostak 2005). Any of these methods can be used to introduce an unnatural amino acid of the type (III), (IV), (VI) or (VII) into the precursor polypeptide for the purpose of generating macrocyclic peptides displayed on an outer biological surface of a host display organism according to the methods disclosed herein.

In some embodiments, the non-canonical amino acid Z (or Z2) is incorporated into the precursor polypeptide via stop codon or four-base codon suppression methods using an engineered AARS/tRNA pair derived from Methanococcus jannaschii tyrosyl-tRNA synthetase (MjTyrRS) and its cognate tRNA (MjtRNA^{Tyr}), an engineered AARS/tRNA pair derived from Methanosarcina berkeri pyrrolysyl-tRNA synthetase (MbPylRS) and its cognate tRNA (tRNA^{Pyl}), an engineered AARS/tRNA pair derived from Methanosarcina mazei pyrrolysyl-tRNA synthetase (MmPylRS) and its cognate tRNA (tRNA^{Pyl}), or an engineered AARS/tRNA pair derived from Escherichia coli tyrosyl-tRNA synthetase (EcTyrRS) and its cognate tRNA (EctRNA^{Tyr}).

In the characterization of the aminoacyl-tRNA synthetase enzymes disclosed herein, these enzymes can be described in reference to the amino acid sequence of a naturally occurring aminoacyl-tRNA synthetase or another engineered aminoacyl-tRNA synthetase. As such, the amino acid residue is determined in the aminoacyl-tRNA synthetase enzyme beginning from the first amino acid after the initial methionine (M) residue (i.e., the first amino acid after the initial methionine M represents residue position 1). It will be understood that the initiating methionine residue may be removed by biological processing machinery such as in a host cell or in vitro translation system, to generate a mature protein lacking the initiating methionine residue. The amino acid residue position at which a particular amino acid or amino acid change is present is sometimes described herein as “Xn”, or “position n”, where n refers to the residue position.

In some embodiments, the stop codon/frameshift codon suppression system used for incorporating the amino acid Z (or Z2) into the precursor polypeptide comprises an engineered variant of Methanococcus jannaschii tRNA^{Tyr} as encoded by a nucleotide of sequence SEQ ID NO: 101, 102, 103, or 104; and an engineered variant of Methanococcus jannaschii tyrosyl-RNA synthetase (SEQ ID NO:31), said variant comprising an amino acid change at least one of the following amino acid positions of SEQ ID NO:31: X32, X63, X65, X70, X107, X108, X109, X155, X158, X159, X160, X161, X162, X163, X164, X167, and X286.

In other embodiments, the stop codon/frameshift codon suppression system used for incorporating the amino acid Z (or Z2) into the precursor polypeptide consists of a Methanococcus jannaschii tRNA^{Tyr} variant selected from the group of tRNA molecules encoded by the nucleotide sequence of SEQ ID NOs: 101, 102, 103, and 104; and a Methanococcus jannaschii tyrosyl-tRNA synthetase variant selected from the group of polypeptides of SEQ ID NOs: 41 through 64.

In some embodiments, the stop codon/frameshift codon suppression system used for incorporating the amino acid Z (or Z2) into the precursor polypeptide comprises an engineered variant of Methanosarcina species tRNA^{Pyl} or Desulfitobacterium hafniense tRNA^{Pyl} as encoded by a nucleotide of sequence SEQ ID NO: 105, 106, 107, 108, 109, 110, 111, or 112; and an engineered variant of Methanosarcina mazei pyrrolysyl-tRNA synthetase (SEQ ID NO:32), said variant comprising an amino acid change at least one of the following amino acid positions of SEQ ID NO:32: X302, X305, X306, X309, X346, X348, X364, X384, X401, X405, and X417.

In some embodiments, the stop codon/frameshift codon suppression system used for incorporating the amino acid Z (or Z2) into the precursor polypeptide comprises an engineered variant of Methanosarcina species tRNA^{Pyl} or Desulfitobacterium hafniense tRNA^{Pyl} as encoded by a nucleotide of sequence SEQ ID NOs: 105, 106, 107, 108, 109, 110, 111, or 112; and an engineered variant of Methanosarcina barkeri pyrrolysyl-tRNA synthetase (SEQ ID NO: 33), said variant comprising an amino acid change at least one of the following amino acid positions of SEQ ID NO:79: X76, X266, X270, X271, X273, X274, X313, X315, and X349.

In other embodiments, the stop codon/frameshift codon suppression system used for incorporating the amino acid Z (or Z2) into the precursor polypeptide consists of a tRNA^{Pyl} variant selected from the group of tRNA molecules encoded by the nucleotide sequence of SEQ ID NOs: 105, 106, 107, 108, 109, 110, 111, and 112; and a pyrrolysyl-tRNA synthetase variant selected from the group of polypeptides of SEQ ID NOs: 65 through 76.

In some embodiments, the stop codon/frameshift codon suppression system used for incorporating the amino acid Z (or Z2) into the precursor polypeptide comprises an engineered variant of Escherichia coli tRNA^{Tyr} or Bacillus stearothermophilus tRNA^{Tyr} as encoded by a nucleotide of sequence SEQ ID NOs: 113, 114, 115, 116, 117, 118, 119, or 120; and an engineered variant of Escherichia coli tyrosyl-tRNA synthetase (SEQ ID NO: 34), said variant comprising an amino acid change at least one of the following amino acid positions of SEQ ID NO: 34: X37, X182, X183, X186, and X265.

In other embodiments, the stop codon/frameshift codon suppression system used for incorporating the amino acid Z (or Z2) into the precursor polypeptide consists of a tRNA Tyr variant selected from the group of tRNA molecules encoded by the nucleotide sequence of SEQ ID NOs: 113, 114, 115, 116, 117, 118, 119, and 120; and a E. coli tyrosyl-tRNA synthetase variant selected from the group of polypeptides of SEQ ID NOs: 77 through 81.

In some embodiments, the expression system used comprises an aminoacyl-tRNA synthetase polypeptide or an engineered variant thereof that is at least 80% identical to any of the polypeptides of SEQ ID NO: 31 to 76; and a transfer RNA molecule encoded by a polynucleotide that is at least 80% identical to any of the polynucleotides of SEQ ID NO:101 to 120. The expression system may comprise an aminoacyl-tRNA synthetase polypeptide or an engineered variant thereof that is at least 90% identical to any of the polypeptides of SEQ ID NO: 31 to 76. The expression system may comprise a transfer RNA molecule encoded by a polynucleotide that is at least 90% identical to any of the polynucleotides of SEQ ID NO:101 to 120. The expression system may also comprise an aminoacyl-tRNA synthetase polypeptide or an engineered variant thereof that is at least 95% identical to any of the polypeptides of SEQ ID NO: 31 to 76. The expression system may also comprise a transfer RNA molecule encoded by a polynucleotide that is at least 95% identical to any of the polynucleotides of SEQ ID NO: 101 to 120.

In some embodiments, the aminoacyl-tRNA synthetase used for incorporating the amino acid Z (or Z2) into the precursor polypeptide can have additionally at least one amino acid residue differences at positions not specified by an X above as compared to the sequence SEQ ID NO: 31, 32, 33, or 34. In some embodiments, the differences can be 1-2, 1-5, 1-10, 1-20, 1-30, 1-40, 1-50, 1-75, 1-100, 1-150, or 1-200 amino acid residue differences at other positions not defined by X above.

In some embodiments, the suppressor tRNA molecule used for incorporating the amino acid Z (or Z2) into the precursor polypeptide can have additionally at least one nucleotide difference as compared to the sequence encoded by the gene of SEQ ID NO: 101, 102, 103, 104, 105, 106, 107, 108, 109, 110, 111, 112, 113, 114, 115, 116, 117, 118, 119, or 120. In some embodiments, the differences can be 1-2, 1-5, 1-10, 1-20, 1-30, 1-40, 1-50, or 1-60 nucleotide differences as compared to the sequences encoded by these genes.

In another embodiment of the method, the engineered variant of Methanococcus jannaschii tyrosyl-tRNA synthetase (SEQ ID NO: 31) comprises at least one of the features selected from the group consisting of: X32 is Tyr, Leu, Ala, Gly, Thr, His, Glu, Val, or Gin; X65 is Leu, His, Tyr, Val, Ser, Thr, Gly, or Glu; X67 is Ala or Gly; X70 is His, Ala, Cys, or Ser; X107 is Glu, Pro, Asn, or Thr; X108 is Phe, Trp, Ala, Ser, Arg, Gly, Tyr, His, Trp, or Glu; X109 is Gln, Met, Asp, Lys, Glu, Pro, His, Gly, Met, or Leu; X155 is Gln, Glu, or Gly; X158 is Asp, Gly, Glu, Ala, Pro, Thr, Ser, or Val; X159 is Ile, Cys, Pro, Leu, Ser, Trp, His, or Ala; X160 is His or Gln; X161 is Tyr or Gly; X162 is Leu, Arg, Ala, Gln, Gly, Lys, Ser, Glu, Tyr, or His; X163 is Gly or Asp; X164 is Val or Ala; X167 is Ala or Val; X286 is Asp or Arg.

In another embodiment of the method, the engineered variant of Methanosarcina mazei pyrrolysyl-tRNA synthetase (SEQ ID NO: 32) comprises at least one of the features selected from the group consisting of: X302 is Ala or Thr; X305 is Leu or Met; X306 is Tyr, Ala, Met, Ile, Leu, Thr, Gly; X309 is Leu, Ala, Pro, Ser, or Arg; X346 is Asn, Ala, Ser, or Val; X348 is Cys, Ala, Thr, Leu, Lys, Met, or Trp; X364 is Thr or Lys; X384 is Tyr or Phe; X405 is Ile or Arg; X401 is Val or Leu; X417 is Trp, Thr or Leu.

In another embodiment of the method, the engineered variant of Methanosarcina barkeri pyrrolysyl-tRNA synthetase (SEQ ID NO: 33) comprises at least one of the features selected from the group consisting of: X76 is Asp or Gly; X266 is Leu, Val, or Met; X270 is Leu or Ile; X271 is Tyr, Phe, Leu, Met, or Ala; X274 is Leu, Ala, Met, or Gly; X313 is Cys, Phe, Ala, Val, or Ile; X315 is Met or Phe; X349 is Tyr, Phe, or Trp.

In another embodiment of the method, the engineered variant of Escherichia coli tyrosyl-tRNA synthetase (SEQ ID NO: 34) comprises at least one of the features selected from the group consisting of: X37 is Tyr, Ile, Gly, Val, Leu, Thr, or Ser; X182 is Asp, Gly, Ser, or Thr; X183 is Phe, Met, Tyr, or Ala; X186 is Leu, Ala, Met, or Val; X265 is Asp or Arg.

An aspect of the methods disclosed herein is the identification and selection of a suitable aminoacyl-tRNA synthetase for incorporating an amino acid Z (or Z2) as defined above, into the artificial precursor polypeptide. Various methods are known in the art to evaluate and quantify the relative efficiency of a given wild-type or engineered aminoacyl-tRNA synthetase to incorporate a non-canonical amino acid into a protein (Young et al. 2011). Any of these methods can be used to guide the identification and choice of a suitable aminoacyl-tRNA synthetase for incorporating a desired amino acid Z (or Z2) into the precursor polypeptide. For example, such efficiency can be measured via a fluorescence assay based on the expression of a reporter fluorescent protein (e.g., green fluorescent protein), whose encoding gene has been modified to contain a codon to be suppressed (e.g., amber stop codon). Expression of the reporter fluorescent protein is then induced in a suitable expression system (e.g., an E. coli or yeast cell) in the presence of the aminoacyl-tRNA synthetase to be tested, a cognate suppressor tRNA (e.g., amber stop codon suppressor tRNA), and the desired non-canonical amino acid. Under these conditions, the relative amount of the expressed (i.e., ribosomally produced) fluorescent protein is linked to the relative efficiency of the aminoacyl-tRNA synthetase to charge the cognate suppressor tRNA with the non-canonical amino acid, which can thus be quantified via fluorimetric means. A demonstration of how this procedure can be applied for selecting an aminoacyl-tRNA synthetase/suppressor tRNA pair for incorporating a desired amino acid Z (or Z2) into the precursor polypeptide is provided in Example 3.

If necessary, the ability of a given aminoacyl-tRNA synthetase/suppressor tRNA pair to incorporate a target non-canonical amino acid into a protein can be improved by means of rational design or directed evolution. While the fluorescence-based method described above can be used to screen several hundreds of engineered aminoacyl-tRNA synthetase variants and/or suppressor tRNA variants for this purpose, higher throughput procedures are also known in the art, which are, for example, based on selection systems (Wu and Schultz 2009; Wang, Xie, and Schultz 2006; Liu and Schultz 2010; Fekner and Chan 2011) or other high-throughput selection procedures (Owens et al. 2017). One such system involves introducing a library of mutated aminoacyl-tRNA synthetases and/or of mutated suppressor tRNAs into a suitable cell-based expression host (e.g., E. coli or yeast cells), whose survival under a suitable selective medium or growth conditions is dependent upon the functionality of the aminoacyl-tRNA synthetase/suppressor tRNA pair. This can be achieved, for example, by introducing a stop codon or four-base codon that is to be suppressed, into a gene encoding for a protein or enzyme essential for survival of the cell, such as a protein or enzyme conferring resistance to an antibiotic. In this case, the ability of the aminoacyl-tRNA synthetase/suppressor tRNA pair to incorporate the desired non-canonical amino acid into the selection marker protein is linked to the survival of the host, thereby enabling the rapid isolation of suitable aminoacyl-tRNA synthetase/suppressor tRNA pair(s) for the incorporation of a particular non-canonical amino acid from very large engineered libraries. The selectivity of these aminoacyl-tRNA synthetase/suppressor tRNA pair toward the desired non-canonical amino acid over the twenty natural amino acids can be further improved by iterative rounds of positive and negative selection as described in (Wu and Schultz 2009; Wang, Xie, and Schultz 2006; Liu and Schultz 2010; Fekner and Chan 2011). Procedures such as those described above can be thus applied to generate and isolate an engineered aminoacyl-tRNA synthetase/suppressor tRNA pair suitable for incorporation of the amino acid Z (or Z2) as defined above, into the precursor polypeptide.

Engineered aminoacyl-tRNA synthetase/tRNA pairs for the incorporation of the amino acid Z (or Z2) into the precursor polypeptide can be prepared via mutagenesis of the polynucleotide encoding for the aminoacyl-tRNA synthetase enzymes of SEQ ID NOs: 31, 32, 33, 34, or an engineered variant thereof; and via mutagenesis of the tRNA-encoding polynucleotides of SEQ ID NOs: 101, 102, 103, 104, 105, 106, 107, 108, 109, 110, 111, 112, 113, 114, 115, 116, 117, 118, 119, 120, or an engineered variant thereof. Many mutagenesis methods are known in the art, and these include, but are not limited to, site-directed mutagenesis, site-saturation mutagenesis, random mutagenesis, cassette-mutagenesis, DNA shuffling, homologous recombination, non-homologous recombination, site-directed recombination, and the like. Detailed description of art-known mutagenesis methods can be found, among other sources, in U.S. Pat. Nos. 5,605,793; 5,830,721; 5,834,252; WO 95/22625; WO 96/33207; WO 97/20078; WO 97/35966; WO 98/27230; WO 98/42832; WO 99/29902; WO 98/41653; WO 98/41622; WO 98/42727; WO 00/18906; WO 00/04190; WO 00/42561; WO 00/42560; WO 01/23401; WO 01/64864.

As described above, the engineered aminoacyl-tRNA synthetases and cognate suppressor tRNA obtained from mutagenesis of SEQ ID NO: 31 to 34, and from mutagenesis of SEQ ID NO: 101 to 120, can be screened for identifying aminoacyl-tRNA synthetase/suppressor tRNA pairs being able, or having improved ability as compared to the corresponding wild-type enzyme/tRNA molecule, to incorporate the amino acid Z (or Z2) into the precursor polypeptide.

In some embodiments, the engineered aminoacyl-tRNA synthetase used in the method comprises an amino acid sequence that is at least 80%, 85%, 90%, 95%, 99% or more identical to the sequence SEQ ID NOs: 31, 32, 33, or 34.

In some embodiments, the engineered suppressor tRNA used in the method is encoded by a polynucleotide comprising a nucleotide sequence that is at least 80%, 85%, 90%, 95%, 99% or more identical to the sequence SEQ ID NOs: 101, 102, 103, 104, 105, 106, 107, 108, 109, 110, 111, 112, 113, 114, 115, 116, 117, 118, 119, or 120.

The target peptide sequence, (AA) n, in the precursor polypeptide of formula (I), (II) and (V) and the second target peptide sequence, (AA)ₒ, in the precursor polypeptide of formula (V), can be a polypeptide comprising 1 to 1,000 amino acid residues. In some embodiments, (AA)ₙ (and (AA)ₒ) consists of a polypeptide comprising 1 to 50 amino acid residues and, in other embodiments, (AA)ₙ (and (AA)ₒ) consists of a polypeptide comprising 1 to 20 amino acid residues.

The N-terminal tail, (AA)ₘ, in the precursor polypeptide of formula (I), (II), and (V) can be a polypeptide comprising 1 to 10,000 amino acid residues. In some embodiments, (AA)ₘ consists of a polypeptide comprising 1 to 1,000 amino acid residues and, in other embodiments, (AA)ₘ consists of a polypeptide comprising 1 to 600 amino acid residues.

The C-terminal tail, (AA)ₚ, in the precursor polypeptide of formula (I), (II), and (V) may not be present, and when present, it can be a polypeptide comprising 1 to 10,000 amino acid residues. When present, (AA)ₘ consists, in some embodiments, of a polypeptide comprising 1 to 1,000 amino acid residues and, in other embodiments, (AA)ₘ consists of a polypeptide comprising 1 to 600 amino acid residues.

The N-terminal tail, (AA)ₘ, the C-terminal tail, (AA)ₚ, or both, in the precursor polypeptides of formula (I), (II), and (V) can comprise a polypeptide affinity tag, a DNA-binding polypeptide, a protein-binding polypeptide, an enzyme, a fluorescent protein, an intein protein, or a combination of these polypeptides.

Introduction of a polypeptide affinity tag within the N-terminal tail and/or C-terminal tail of the precursor polypeptide results in cyclopeptibody fused to such polypeptide affinity tag. Such affinity tags can be useful for isolating, purifying, and/or immobilizing onto a solid support the macrocyclic peptides generated according to the methods disclosed herein. Accordingly, in some embodiments, the N-terminal tail, C-terminal tail, or both, of the precursor polypeptides comprise at least one polypeptide affinity tags selected from the group consisting of a polyarginine tag (e.g., RRRRR) (SEQ ID NO:121), a polyhistidine tag (e.g., HHHHHH) (SEQ ID NO: 122), an Avi-Tag (SGLNDIFEAQKIEWHELEL) (SEQ ID NO:123), a FLAG tag (DYKDDDDK) (SEQ ID NO:124), a Strep-tag II (WSHPQFEK) (SEQ ID NO:125), a c-myc tag (EQKLISEEDL) (SEQ ID NO:126), a S tag (KETAAAKFERQHMDS) (SEQ ID NO:127), a calmodulin-binding peptide (KRRWKKNFIAVSAANRFKKISSSGAL) (SEQ ID NO:128), a streptavidin-binding peptide (MDEKTTGWRGGHVVEGLAGELEQLRARLEHHPQGQREP) (SEQ ID NO:129), a chitin-binding domain (SEQ ID NO:130), a glutathione S-transferase (GST; SEQ ID NO:131), a maltose-binding protein (MBP; SEQ ID NO:132), streptavidin (SEQ ID NO: 133), and engineered variants thereof. These aspects are illustrated in Example 2.

The N-terminal tail, (AA)ₘ, the C-terminal tail, (AA)ₚ, or both, in the precursor polypeptides of formula (I), (II), and (V) can comprise a reporter protein or enzyme. This approach will result in the formation of cyclopeptibodies fused to a reporter protein or enzyme, which can be useful to facilitate the functional screening of said macrocyclic peptides. In some embodiments, the N-terminal tail, (AA)ₘ and/or the C-terminal tail, (AA)ₚ, in the precursor polypeptides of formula (I), (II), and (V) comprise at least one polypeptide selected from the group consisting of green fluorescent protein (SEQ ID NO:134), luciferase (SEQ ID NO:135), alkaline phosphatase (SEQ ID NO:136), and engineered variants thereof.

The N-terminal tail, (AA)ₘ, the C-terminal tail, (AA)ₚ, or both, in the precursor polypeptides of formula (I), (II), or (V) can comprise a protein or enzyme that is part of a display system such as, for example, a phage display (e.g. M13, T7, or lambda phage display), a yeast display, a bacterial display, a DNA display, a plasmid display, a CIS display, a ribosome display, or a mRNA display system. This approach can be useful for generating libraries of cyclopeptibodies which are physically linked to, or compartmentalized with the polynucleotide sequence that encodes for the corresponding precursor polypeptides. In turn, this approach can be useful toward isolating functional cyclopeptibodies that are able to bind, inhibit or activate a certain target biomolecule (e.g., protein, enzyme, DNA or RNA molecule) or target biomolecular interaction.

The immunoglobulin Fc region may be derived from humans or animals (mammals) such as cattle, goats, pigs, mice, rabbits, hamsters, rats, guinea pigs, and the like. In some embodiments, the immunoglobulin Fc region comprised in the cyclopeptibody molecule is derived from human IgG, IgA, IgM, IgD, or IgE. In some embodiments, the immunoglobulin Fc region comprised in the cyclopeptibody molecule is derived from mouse IgG, IgA, IgM, or IgE. In some embodiments, the immunoglobulin Fc region comprised in the cyclopeptibody molecule is derived from human IgG1 (SEQ ID NO: 1), IgG2 (SEQ ID NO: 2), IgG3 (SEQ ID NO: 3) or IgG4 (SEQ ID NO: 4). In some embodiments, the Fc domain is an engineered variant of human IgG1 of SEQ ID NO: 1 which comprises an amino acid substitution at a position selected from the group consisting of position: X351, X366, X368, X382, X395, X409, X428, and X434 in the corresponding full-length IgG1.

In some embodiments, the immunoglobulin Fc region comprised in the cyclopeptibody molecule is derived from mouse IgG1 (SEQ ID NO: 5) or mouse IgG2 (SEQ ID NO: 6).

In some embodiments, the immunoglobulin Fc region in the cyclopeptibody molecule is a polypeptide that comprises the heavy chain constant region 2 (CH₂), heavy chain constant region 3 (CH₃) and hinge region derived from human IgG1 (SEQ ID NO: 1), human IgG2 (SEQ ID NO: 2), human IgG3 (SEQ ID NO: 3), or human IgG4 (SEQ ID NO: 4).

In other embodiments, the immunoglobulin Fc region in the cyclopeptibody molecule is a polypeptide that comprises the heavy chain constant region 2 (CH₂), heavy chain constant region 3 (CH₃) and hinge region derived from mouse IgG1 represented by SEQ ID NO: 5 or derived from mouse IgG2 represented by SEQ ID NO: 6.

The immunoglobulin Fc region may contain carbohydrate (oligosaccharide) groups that are attached to sites known to be glycosylation sites in proteins. Generally, O-linked oligosaccharides are attached to serine (Ser) or threonine (Thr) residues while N-linked oligosaccharides are attached to asparagine (Asn) residues when they are part of the sequence Asn-X-Ser/Thr, where X can be any of the naturally occurring amino acid except proline. The structures of N-linked and O-linked oligosaccharides and the sugar residues found in type are different. One type of sugar that is commonly found on both is N-acetylneuraminic acid (referred to as sialic acid). Sialic acid is usually the terminal residue of both N-linked and O-linked oligosaccharides and, by virtue of its negative charge, may confer acidic properties to the glycosylated compound. The glycosylation site(s) in the Fc domain are preferably glycosylated by a cell during recombinant production of the polypeptide compounds (e.g., in mammalian cells such as CHO, BHK, COS). However, such sites may further be glycosylated by synthetic or semi-synthetic procedures known in the art.

The immunoglobulin Fc region may be in the native glycosylated form, in alternative glycosylated forms, in which the sugar chain is increased or reduced compared to the natural form, or in a deglycosylated form, in which the sugar chain is removed from the natural form. The immunoglobulin Fc region may be also in an aglycosylated form, in which the sugar chain is not produced during recombinant expression as in the case of expression in prokaryotes, such as for example Escherichia coli. Conventional methods such as chemical methods, enzymatic methods, and genetic engineering methods using microorganisms can be used to increase or decrease such the sugar chains in immunoglobulin molecules. In general, immunoglobulin Fc regions in which the sugar chain is removed from the Fc show reduced binding toward the complement (c1q), resulting in reduced antibody-dependent cytotoxicity or complement-dependent cytotoxicity. Depending on the application, the latter may or may not be desirable to reduce or increase an immune response in vivo.

In some embodiments, the immunoglobulin Fc region comprised in the cyclopeptibody molecule is aglycosylated. In some embodiments, the Fc region comprised in the cyclopeptibody molecule is glycosylated. In some embodiments, the Fc region comprised in the cyclopeptibody molecule is deglycosylated.

In some embodiments, the Fc region comprised in the cyclopeptibody molecule is a native Fc domain which corresponds to the Fc domain found in the corresponding immunoglobin from the organism of origin. In some embodiments, the Fc region comprised in the cyclopeptibody molecule is a variant, an analog, an engineered mutant, a hybrid, a truncation, a fragment, or a derivative of a human Fc domain or of an alternative Fc domain polypeptide.

The Fc region comprised in the cyclopeptibody molecule may be an engineered variant of an immunoglobulin Fc domain. The Fc variant may be a molecule or sequence that is modified from a native Fc but still comprises a binding site for the salvage receptor, FcRn, as described in WO 97/34631 and WO 96/32478. The Fc variant may also be a sequence that is humanized from a non-human native Fc. Furthermore, the Fc domain may be an engineered Fc variant, in which native Fc sites are modified or removed because they provide structural features or biological activity that are not required for molecules of the present invention. Many of these modifications are known in the art, as described for example, in Feige et al, U.S. Pat. No. 6,660,843B1).

Exemplary Fc variants include molecules and sequences in which:
1. Sites involved in disulfide bond formation are removed. Such removal may avoid reaction with other cysteine-containing proteins present in the host cell used to produce the molecules of the invention. For this purpose, the cysteine-containing segment at the N-terminus may be truncated or cysteine residues may be deleted or substituted with other amino acids (e.g., Ala, Ser). Even when cysteine residues are removed, the single chain Fc domains can still form a dimeric Fc domain that is held together non-covalently.
2. A native Fc is modified to make it more compatible with a selected host cell. For example, one may remove the PA sequence near the N-terminus of a typical native Fc, which may be recognized by a digestive enzyme in E. coli such as proline iminopeptidase. One may also add an N-terminal methionine residue, especially when the molecule is expressed recombinantly in a bacterial cell such as E. coli.
3. A portion of the N-terminus of a native Fc is removed to prevent N-terminal heterogeneity when expressed in a selected host cell. For this purpose, one may delete any of the first 20 amino acid residues at the N-terminus, particularly those at positions 1, 2, 3, 4 and 5.
4. One or more glycosylation sites are removed. Residues that are typically glycosylated (e.g., asparagine) may confer cytolytic response. Such residues may be deleted or substituted with unglycosylated residues (e.g., alanine).
5. Sites involved in interaction with complement, such as the C1q binding site, are removed. For example, one may delete or substitute the EKK sequence of human IgG1. Complement recruitment may not be advantageous for the molecules of this invention and so may be avoided with such an Fc variant.
6. Sites are removed that affect binding to Fc receptors other than a salvage receptor. A native Fc may have sites for interaction with certain white blood cells that are not required for the fusion molecules of the present invention and so may be removed.
7. The ADCC site is removed. ADCC sites are known in the art; see, for example, Molec. Immunol. 29 (5):633-9 (1992) with regard to ADCC sites in IgG1. These sites, as well, are not required for the fusion molecules of the present invention and so may be removed.
8. When the native Fc is derived from a non-human antibody, the native Fc may be humanized. Typically, to humanize a native Fc, one will substitute selected residues in the non-human native Fc with residues that are normally found in human native Fc. Techniques for antibody humanization are well known in the art.

In some embodiments, the Fc domain comprised in the cyclopeptibody molecule is an engineered variant of an immunoglobulin Fc domain, in which one or more amino acid mutations have been introduced to impact its stability, dimerization ability, FcRn binding properties, glycosylation, expression level, its ability to interact with complement, its ADCC sites, expression level in the producing host, or any combination of these properties.

In some embodiments, the engineered Fc domain variant used in the method comprises an amino acid sequence that is at least 80%, 85%, 90%, 95%, 99% or more identical to the sequence SEQ ID NOs: 1, 2, 3, 4, 5, 7, 8, or 9.

In some embodiments, the Fc region comprised in the cyclopeptibody molecule is an engineered variant of a human IgG1 Fc domain, in which one or more of the amino acid residues corresponding to L351, T366, L368, E382, P395, K409, M428, and N434 (where residue numbering corresponds to that of full-length human IgG1) have been mutated. Mutations at these positions are known to impact the dimerization or FcRn binding properties of human IgG1 Fc domain (Roopenian et al., Nat Rev Immunol (2007) 7(9):715-25; Vaccaro et al, Nat Biotechnol (2005) 23(10):1283-8; Ying et al. J Biol Chem (2012) 287(23):19399-408; Ying et al, mAbs (2014) 6(5):1201-10; Martin et al. Mol Cell (2001) 7(4):867-77; Wang et al. Front Immunol. (2017) 8:1545). Moreover, mutations in the Fc domain that enhance FcRn binding can be useful for elongating half-life or increasing cellular uptake of IgG (Roopenian et al., Nat Rev Immunol (2007) 7(9):715-25; Ying et al. Biochim Biophys Acta (2014) 1844(11):1977-82; Simister et al., Nature (1989) 337(6203):184-7; Zalevsky et al, Nat Biotechnol. (2010) 28(2):157-9). In some embodiments, the Fc region comprised in the cyclopeptibody molecule is an engineered variant of a human IgG1 Fc domain containing the amino acid mutations E382V and M428I.

The immunoglobulin Fc region comprised in the cyclopeptibody molecule may be a “hybrid Fc domain” as defined herein. The hybrid Fc domain may be derived from a combination of a heavy chain constant region 2 (CH2), a heavy chain constant region 3 (CH3), and a hinge region derived from different classes or subclasses of immunoglobulins, these immunoglobulins being derived from the same organism or different organisms. For example, the hybrid Fc domain may be derived from a combination of a heavy chain constant region 2 (CH2), a heavy chain constant region 3 (CH3), and hinge region derived from different human IgG subclasses or a combination of human IgD and IgG. As another example, the hybrid Fc may include an IgD hinge region and a heavy chain constant region 2 (CH2) N-terminal region fused to an IgG4 CH2 and CH3 region as described in Korean Patent No. 0897938. The use of hybrid Fc domains may be useful to increase the serum half-life of the Fc domain-fused protein and/or increase the expression level of the polypeptide. In some embodiments, the Fc domain comprised in the cyclopeptibody molecule is a hybrid Fc domain derived from a combination of human IgG subclasses. In other embodiments, the Fc domain comprised in the cyclopeptibody molecule is a hybrid Fc domain derived from a combination of human IgD and IgG.

The cyclic peptide in the cyclopeptibody molecule may be genetically fused to the N-terminal end or to the C-terminal end of the immunoglobulin Fc domain or a fragment thereof. In some embodiments, the cyclic peptide is fused to the N-terminal end of the Fc domain or fragment thereof. In other embodiments, the cyclic peptide is fused to the C-terminal end of the Fc domain or fragment thereof. Accordingly, in some embodiments, the Fc domain, or fragment thereof, is comprised within the N-terminal tail, (AA)ₘ, of the precursor polypeptides of formula (I), (II), and (V). In other embodiments, the Fc domain, or fragment thereof, is comprised within the c-terminal tail, (AA)ₚ, of the precursor polypeptides of formula (I), (II), and (V).

A polypeptide sequence may be introduced as a “spacer sequence” between the cyclic peptide and the Fc domain, or fragment thereof, in the cyclopeptibody molecule. This spacer sequence may be chosen so that the fusion does not compromise the function of the cyclic peptide (e.g., binding to a target protein of interest) and/or of the Fc domain (e.g., FcRn binding), or to enhance the stability and/or protein binding affinity of the cyclopeptibody. In some embodiments, the spacer sequence is 1 to 30 or more amino acids in length and can be a small neutral polar or non-polar amino acid such as glycine, cysteine, serine, or threonine. Exemplary spacer sequences have an amino acid sequence corresponding to (Gly-Ser)₁₋₁₀, (Gln)₆, (Pro-Ala-Ser)₂, (Asn-Xaa-Xaa)₁₋₄, or (Lys-Xaa-Xaa)₁₋₄, wherein Xaa is a small neutral polar or nonpolar amino acid. The linkers shown here are exemplary; linkers within the scope of this invention may be much longer and may include other residues. In some embodiments, the linker is cleavable, for example, to facilitate separation of cyclic peptide from the Fc region. In some embodiments, a protease cleavage site is included between the cyclic peptide and the Fc domain.

In some embodiments, the cyclopeptibody molecule is a ‘polyvalent’ cyclopeptibody, which contains two or more copies of the same cyclic peptide fused to the N-terminal end or to the C-terminal end of the immunoglobulin Fc domain or a fragment thereof. The presence of multiple copies of the same cyclic peptide targeted against a target protein of interest can provide an enhancement in affinity and/or specificity of cyclopeptibody molecule toward the target protein of interest due to avidity effects.

In some embodiments, the cyclopeptibody molecule is a ‘bispecific’ cyclopeptibody, which contains two different cyclopeptides that are able to specifically recognize two different target proteins of interest. Bispecific cyclopeptibody can be designed and prepared in different formats such as, for example, by fusing one of the two cyclopeptides to the N-terminus of the immunoglobulin Fc domain or a fragment thereof, and the other cyclopeptide to the C-terminus of the immunoglobulin Fc domain or a fragment thereof. In another format, two cyclic peptides with different specificity can be fused in tandem to the N-terminus or the C-terminus of the immunoglobulin Fc domain or a fragment thereof. In yet another format, a ‘bispecific’ cyclopeptibody can be prepared through the association of two immunoglobulin Fc domains, each containing a N-terminally or C-terminally fused cyclic peptide with different protein binding specificity. In this case, the two immunoglobulin Fc domains can be chosen to contain mutations (e.g., so-called knob/hole mutations) that facilitate heterodimer formation over homodimer formation as illustrated in Example 15. Similarly, ‘multispecific’ cyclopeptibody molecules can be prepared by fusing three or more cyclic peptides with different protein binding specificity to an immunoglobulin Fc domain or a fragment thereof. Bispecificity can be useful for many therapeutic applications as illustrated by FDA-approved therapeutic bispecific antibodies such as, for example, Blitanunomab, Catumaxomab, and Emixizumab.

In some embodiments, the bispecific cyclopeptibody molecule contains two cyclopeptides with different target specificity fused in tandem to the N-terminus or the C-terminus of the immunoglobulin Fc domain or a fragment thereof. In other embodiments, the bispecific cyclopeptibody molecule derives from the association of two immunoglobulin Fc domains or fragments thereof, each containing a N-terminally or C-terminally fused cyclic peptide with different protein binding specificity. The polypeptide of any of claims 1-22, wherein the cyclic peptide is able to bind to a polypeptide, nucleic acid, or carbohydrate molecule.

In some embodiments, the cyclic peptide is able to bind to Programmed Death-Ligand 1, a Hedgehog protein, or an integrin protein. In certain embodiments, the polypeptide is at least 80%, 90%, 95% or 99% identical to SEQ ID NO: 205, 206, 207, 208, 209, 0210, 211, 212, 213, 214, 215, 216, 217, 218, 219, 220, 221, 222, 223, 224, 225, 226, 227, 228, 229, 230, 231, 232, 233, 234, 239 or 240.

In some embodiments, wherein the cyclic peptide is able to bind to Kelch-like ECH-associated protein 1 (Keap1) or streptavidin. In certain embodiments, the polypeptide is at least 80%, 90%, 95% or 99% identical to SEQ ID NO: 201, 202, 203, 204, 235, 236, 237, 238, 239 or 240.

The disclosed cyclic peptides may be used as an active ingredient in a pharmaceutical composition for the prevention or treatment of cancer comprising the cyclic peptide. The disclosed cyclic peptides may also be used as a tumor imaging and/or detection agent. The disclosed cyclic peptides may also be used as an active ingredient in a protein-detection agent. The disclosed cyclopeptide bodies may be included in a kit for detection of Programmed Death-Ligand 1, Hedgehog, or integrin comprising the cyclic peptide. The disclosed cyclic peptides may be included as a reagent in a kit for labelling a receptor comprising the cyclic peptide or for detecting a protein comprising the cyclic peptide according to claim 28-31 as a reagent.

As noted above, multiple copies of the same cyclic peptide may be fused to the Fc region of an immunoglobulin molecule or fragment thereof, resulting in a polyvalent cyclopeptibody. In another embodiment, two or more cyclic peptides with different binding specificity may be fused to the Fc region of an immunoglobulin molecule or fragment thereof, resulting in a polyspecific cyclopeptibody. The cyclopeptibody molecule may be a heterodimeric bispecific cyclopeptibody. The cyclopeptibody may be able to bind to Kelch-like ECH-associated protein 1 (Keap1) and streptavidin, or to streptavidin and PD-L1. In certain embodiments, the polypeptide is at least 80%, 90%, 95% or 99% identical to SEQ ID NO: 237 or 238. In certain embodiments, the polypeptide is at least 80%, 90%, 95% or 99% identical to SEQ ID NO: 239 or 240.

### 5.3 Polynucleotides and Host Cells for Production of Cyclopeptibodies

In another aspect, polynucleotide molecules are provided encoding for precursor polypeptides of formula (I), (II), and (V) as defined above. Polynucleotide molecules are provided for encoding for the aminoacyl-tRNA synthetases and cognate tRNA molecules for the ribosomal incorporation of the amino acid Z into the precursor polypeptides of formula (I) and (II) and for the ribosomal incorporation of the amino acid Z2 into the precursor polypeptides of formula (V). Polynucleotide molecules are provided encoding for polypeptide sequences that can be introduced within the N-terminal tail ((AA)ₘ) or C-terminal tail ((AA)ₚ) of the precursor polypeptides of formula (I), (II) and (V), such as Fc domains, peptide and protein affinity tags, reporter proteins and enzymes, as described above. Since the correspondence of all the possible three-base codons to the various amino acids is known, providing the amino acid sequence of the polypeptide provides also a description of all the polynucleotide molecules encoding for such polypeptide. Thus, a person skilled in the art will be able, given a certain polypeptide sequence, to generate any number of different polynucleotides encoding for the same polypeptide. In some embodiments, the codons are selected to fit the host cell in which the polypeptide is being expressed. For example, codons used in bacteria can be used to express the polypeptide in a bacterial host. The polynucleotides may be linked to one or more regulatory sequences controlling the expression of the polypeptide-encoding gene to form a recombinant polynucleotide capable of expressing the polypeptide.

Numerous methods for making nucleic acids encoding for polypeptides having a predetermined or randomized sequence are known to those skilled in the art. For example, oligonucleotide primers having a predetermined or randomized sequence can be prepared chemically by solid phase synthesis using commercially available equipment and reagents. Polynucleotide molecules can then be synthesized and amplified using a polymerase chain reaction, digested via endonucleases, ligated together, and cloned into a vector according to standard molecular biology protocols known in the art (e.g., Sambrook et al. Molecular Cloning: A Laboratory Manual (Third Edition), Cold Spring Harbor Press, 2001). These methods, in combination with the mutagenesis methods mentioned above, can be used to generate polynucleotide molecules that encode for the aforementioned polypeptides as well as suitable vectors for the expression of these polypeptides in a host expression system.

The precursor polypeptides can be produced by introducing said polynucleotides into an expression vector, by introducing the resulting vectors into an expression host, and by inducing the expression of the encoded precursor polypeptides in the presence of the amino acid Z (or Z2) and, whenever necessary, also in the presence of a suitable stop codon or frameshift codon suppression system for mediating the incorporation of the amino acid Z (or Z2) into the precursor polypeptides.

Nucleic acid molecules can be incorporated into any one of a variety of expression vectors suitable for expressing a polypeptide. Suitable vectors include, but are not limited to, chromosomal, nonchromosomal, artificial and synthetic DNA sequences, e.g., derivatives of SV40; bacterial plasmids; phage DNA; baculovirus; yeast plasmids; vectors derived from combinations of plasmids and phage DNA, viral DNA such as vaccinia, adenovirus, fowl pox virus, pseudorabies, adenovirus, adeno-associated viruses, retroviruses and many others. Any vector that transduces genetic material into a cell, and, if replication is desired, which is replicable and viable in the relevant host can be used. A large number of expression vectors and expression hosts are known in the art, and many of these are commercially available. A person skilled in the art will be able to select suitable expression vectors for a particular application, e.g., the type of expression host (e.g., in vitro systems, prokaryotic cells such as bacterial cells, and eukaryotic cells such as yeast, insect, or mammalian cells) and the expression conditions selected.

Expression hosts that may be used for the preparation of the precursor polypeptides and cyclopeptibodies include, but are not limited to, any systems that support the transcription, translation, and/or replication of a nucleic acid. In some embodiments, the expression host system is a cell or a cell lysate. Host cells for use in expressing the polypeptides encoded by the expression vector of this disclosure are well known in the art and include, but are not limited to, bacterial cells (e.g., Escherichia coli, Streptomyces sp., Bacillus sp.); fungal cells, including yeast cells (e.g., Saccharomyces cerevisiae, Pichia pastoris); insect cells; plant cells; and animal cells, such as mammalian cells (e.g., NS0 murine myeloma cells, Chinese hamster ovary (CHO) cells, baby hamster kidney cells (BHK), COS cells) and human cells (e.g., PER.C6® human cells). Other expression systems include lysates of prokaryotic cells (e.g., bacterial cells) and lysates of eukaryotic cells (e.g., yeast, insect, or mammalian cells). In preferred embodiments, the expression host system is a cell.

In some embodiments, the expression host system is a bacterial cell such as a E. coli cell. In other embodiments, the expression host system is a yeast cell such as a Saccharomyces cerevisiae or Pichia pastoris cell. In other embodiments, the expression host system are mammalian cells selected from immortal hybridoma cells, NS/O myeloma cells, 293 cells, Hela cells, COS cells, PER.C6® human cells, or Chinese hamster ovary (CHO) cells.

The choice of the expression vector and host expression system depends on the type of application intended for the methods disclosed herein and a person skilled in the art will be able to select a suitable expression host based on known features and application of the different expression hosts and host display organisms. For example, bacterial cells (e.g., E. coli) can be used to produce aglycosylated cyclopeptibodies, whereas yeast, insect, or mammalian cell hosts can be used produce glycosylated cyclopeptibodies. In particular, mammalian cell hosts can be used to produce glycosylated cyclopeptibodies containing sugar chains that correspond to, or are most similar, to the sugar chains found in the native Fc domain.

In some embodiments, the cyclopeptibody is produced within a cell-free expression system. This method comprises providing a nucleic acid encoding for the precursor polypeptide, introducing the nucleic acid into the cell-free expression host, inducing the expression of the precursor polypeptide, allowing for the precursor polypeptide to undergo intramolecular cyclization via a bond-forming reaction between the side-chain sulfhydryl group of the cysteine and the FG₁ group of the amino acid Z (or between the cysteines and the FG₁ and FG₂ groups of the amino acid Z2), thereby producing the cyclopeptibody within the cell-free expression host.

A method is also provided for making a library of cyclopeptibodies via cyclization of a plurality of precursor polypeptides of formula (I) or (II) that contain a heterogeneous peptide target sequence (AA) n, or a heterogeneous N-terminal tail (AA)ₘ, or a heterogeneous C-terminal tail (AA)ₚ, or a combination of these, wherein the at least one of (AA)ₚ and (AA)ₘ comprises the Fc region of an immunoglobulin molecule or fragment thereof. This method comprises: (a) constructing a plurality of nucleic acid molecules encoding for a plurality of precursor polypeptides, said precursor polypeptides having an heterogeneous peptide target sequence (AA) n, or an heterogeneous N-terminal tail (AA)ₘ, or an heterogeneous C-terminal tail (AA)ₚ, or a combination of these; (b) introducing each of the plurality of said nucleic acid molecules into an expression vector, and introducing the resulting vectors into an expression host; (c) expressing the plurality of precursor polypeptides; (d) allowing for the precursor polypeptides to undergo intramolecular cyclization via a bond-forming reaction between the side-chain sulfhydryl group of the cysteine and the FG₁ group of the amino acid Z, thereby producing a plurality of cyclopeptibody molecules.

A method is also provided for making a library of cyclopeptibodies via cyclization of a plurality of precursor polypeptides of formula (V) that contain a heterogeneous peptide target sequence (AA)ₙ, or a heterogeneous second peptide target sequence (AA)ₒ, or a heterogeneous N-terminal tail (AA)ₘ, or a heterogeneous C-terminal tail (AA)ₚ, or a combination of these, wherein the at least one of (AA)ₚ and (AA)ₘ comprises the Fc region of an immunoglobulin molecule or fragment thereof. This method comprises: (a) constructing a plurality of nucleic acid molecules encoding for a plurality of precursor polypeptides, said precursor polypeptides having an heterogeneous peptide target sequence (AA) n, or an heterogeneous second peptide target sequence (AA)ₙ, or an heterogeneous N-terminal tail (AA)ₘ, or an heterogeneous C-terminal tail (AA)ₚ, or a combination of these; (b) introducing each of the plurality of said nucleic acid molecules into an expression vector, and introducing the resulting vectors into an expression host; (c) expressing the plurality of precursor polypeptides; (d) allowing for the precursor polypeptides to undergo intramolecular cyclization via a bond-forming reaction between the side-chain sulfhydryl group of the cysteines and the FG₁ and FG₂ group2 of the amino acid Z2, thereby producing a plurality of cyclopeptibody molecules.

In specific embodiments, each of the plurality of cyclopeptibodies prepared as described above is tethered to a cell component, to a cell wall component, to a cell membrane component, to a bacteriophage, to a viral particle, or to a DNA molecule, via a polypeptide comprised within the N-terminal tail or within the C-terminal tail of said macrocyclic peptide molecule.

Several methods of making polynucleotides encoding for heterogeneous peptide sequences are known in the art. These include, among many others, methods for site-directed mutagenesis (Botstein, D.; Shortle, D. Science (New York, N.Y, 1985, 229, 1193; Smith, M. Annual review of genetics, 1985, 19, 423; Dale, S. J.; Felix, I. R. Methods in molecular biology (Clifton, N.J, 1996, 57, 55; Ling, M. M.; Robinson, B. H. Analytical biochemistry, 1997, 254, 157), oligonucleotide-directed mutagenesis (Zoller, M. J. Current opinion in biotechnology, 1992, 3, 348; Zoller, M. J.; Smith, M. Methods Enzymol, 1983, 100, 468; Zoller, M. J.; Smith, M. Methods Enzymol, 1987, 154, 329), mutagenesis by total gene synthesis and cassette mutagenesis (Nambiar, K. P.; Stackhouse, J.; Stauffer, D. M.; Kennedy, W. P.; Eldredge, J. K.; Benner, S. A. Science (New York, N.Y, 1984, 223, 1299; Grundstrom, T.; Zenke, W. M.; Wintzerith, M.; Matthes, H. W.; Staub, A.; Chambon, P. Nucleic acids research, 1985, 13, 3305; Wells, J. A.; Vasser, M.; Powers, D. B. Gene, 1985, 34, 315), and the like. Additional methods are described in the following U.S. patents, PCT publications, and EPO publications: U.S. Pat. No. 5,605,793 “Methods for In vitro Recombination”, U.S. Pat. No. 5,830,721 “DNA Mutagenesis by Random Fragmentation and Reassembly”, WO 95/22625 “Mutagenesis by Random Fragmentation and Reassembly”, WO 96/33207 “End Complementary Polymerase Chain Reaction”, EP 752008 “DNA Mutagenesis by Random Fragmentation and Reassembly”, WO 98/27230 “Methods and Compositions for Polypeptide Engineering”, WO 00/00632, “Methods for Generating Highly Diverse Libraries”, WO 98/42832 “Recombination of Polynucleotide Sequences Using Random or Defined Primers”, WO 99/29902 “Method for Creating Polynucleotide and Polypeptide Sequences”. Any of these methods or modifications thereof can be utilized for generating nucleotide molecules that encode for precursor polypeptides of formula (I), (II), or (V) which are fused to an immunoglobulin Fc region and which contain a heterogeneous peptide target sequence (AA) n, a heterogeneous second peptide target sequence (AA)ₒ, a heterogeneous N-terminal tail (AA)ₘ, a heterogeneous C-terminal tail (AA)ₚ, or a combination of these, for the purpose of generating a library of cyclopeptibodies. The library of cyclopeptibodies can be screened to select for cyclopeptibodies that exhibit improved binding affinity toward a protein of interest or other properties of interest.

The compounds provided herein may contain one or more chiral centers. Accordingly, the compounds are intended to include, but not be limited to, racemic mixtures, diastereomers, enantiomers, and mixture enriched in at least one stereoisomer or a plurality of stereoisomers. When a group of substituents is disclosed herein, all the individual members of that group and all subgroups, including any isomers, enantiomers, and diastereomers are intended to be included in the disclosure. Additionally, all isotopic forms of the compounds disclosed herein are intended to be included in the disclosure. For example, it is understood that any one or more hydrogens in a molecule disclosed herein can be replaced with deuterium or tritium.

### 5.4 Uses of Cyclopeptibody Molecules

The cyclopeptibody molecules of this invention can have pharmacologic activity resulting from their ability to bind to proteins of interest as agonists, mimetics or antagonists of the native ligands of such proteins of interest. In addition, the cyclopeptibody molecules of this invention can have pharmacologic activity resulting from their ability to bind to proteins of interest in a site of these proteins that is distinct from that of the native ligands of such proteins of interest, such as, for example, an allosteric site. The utility of specific cyclopeptibody molecules is shown in Table 1 and further illustrated in Examples 5-10. The activity of these cyclopeptibody molecules can be measured by assays known in the art, such as in vitro assay, cell-based assays, in vivo activity experiments, and the like.

In general, a biologically or pharmacologically active cyclopeptibody molecule can be prepared by genetically fusing an immunoglobin Fc domain to a cyclic peptide that is generated according to the methods described herein and that is capable of binding a target biomolecule of interest. This target biomolecule can be a protein, a nucleic acid molecule (e.g., DNA or RNA molecule), a carbohydrate molecule, a lipid molecule, or a small molecule. As a result of this fusion procedure, the cyclopeptibody molecule is conferred with the ability of binding such target molecule, often albeit not necessarily with improved binding affinity compared to the cyclic peptide alone, while possessing other favorable attributes such as prolonged in vivo half-life and/or other functions mediated by the Fc domain comprised within the cyclopeptibody. In some embodiments, the biomolecule targeted by the cyclopeptibody molecule is a protein. In other embodiments, the biomolecule targeted by the cyclopeptibody molecule is a nucleic acid molecule. In other embodiments, the biomolecule targeted by the cyclopeptibody molecule is a carbohydrate molecule.

In addition to therapeutic uses, the cyclopeptibody molecules of the present invention are useful in diagnosing diseases characterized by dysfunction of their associated protein of interest. In one embodiment, a method of detecting in a biological sample a protein of interest (e.g., a receptor) that is capable of being bound by the cyclopeptibody molecule, comprises the steps of: (a) contacting the sample with a cyclopeptibody molecule of this invention; and (b) detecting binding of the protein of interest by the cyclopeptibody molecule. The biological samples include tissue specimens, intact cells, or extracts thereof. The cyclopeptibody molecules of this invention may be used as part of a diagnostic kit to detect the presence of their associated proteins of interest in a biological sample. Such kits employ the cyclopeptibody molecules of the invention having an attached label (e.g., fluorescent label, affinity label) to allow for detection.

In these applications, the cyclopeptibody molecules can be useful for identifying normal or abnormal proteins of interest in the biological sample.

As illustrative examples, but without being limited by them, the inventors have determined cyclopeptibody sequences having many different kinds of activity, such as the ability of binding streptavidin, human Kelch-like ECH-associated protein 1 (Keap1), a human Hedgehog protein (i.e. Sonic, Desert, and/or Indian Hedgehog), human Programmed Death-Ligand 1 (PD-L1), and a human integrin receptor (i.e., αᵥβ₃ integrating receptor). Exemplary structures for these cyclopeptibody molecules are listed in Table 1.

### 5.5 Therapeutic Uses of Cyclopeptibody Molecules

The Hedgehog-binding, PD-L1-binding, and the integrin-binding cyclopeptibodies described herein may be used in the treatment or prevention of neoplasms or other proliferative diseases in a patient affected by such disease(s). As used herein, the terms “cancer”, “proliferative disease” and “neoplasms” refer to a condition in which cells have the capacity for autonomous growth, i.e., an abnormal state or condition characterized by rapidly proliferating cell growth. Proliferative and neoplastic disease states may be categorized as pathologic, i.e., characterizing or constituting a disease state, or may be categorized as non-pathologic, i.e., a deviation from normal but not associated with a disease state. In certain embodiments, the neoplasm is a benign neoplasm. In other embodiments, the neoplasm is a malignant neoplasm. These compounds are administered by an amount and route of delivery that is appropriate for the nature and severity of the condition being treated and may be ascertained by one skilled in the art. Preferably, administration is by injection, either subcutaneous, intramuscular, or intravenous.

The Hedgehog-binding cyclopeptibodies of the invention may be particularly useful for the treatment of diseases or conditions whose development or progression is associated with physiological or aberrant stimulation of the Hedgehog pathway. In some embodiments, compounds of the present invention are used to inhibit the signaling function of one or more homologs of Hedgehog, i.e., Shh, Ihh, and/or Dhh. In some embodiments, the compounds of the present invention are used to inhibit the Hedgehog pathway in cells. In some embodiments, the cells are wild type. In some embodiments, the cells are subject to one or more genetic modifications. In some embodiments, the cells are cancer cells.

The Hedgehog-binding cyclopeptibodies of the invention may be useful as therapeutic agents, also as part of a combination therapy, for the treatment of pathologies associated with ligand-dependent stimulation of the Hedgehog pathway (Yauch et al. 2008; Theunissen and de Sauvage 2009; Tian et al. 2009). Such pathologies include, but are not limited to, hematopoietic neoplastic disorders (e.g., leukemia, lymphoma), solid cancers (e.g., colon cancer, lung cancer, bone cancer, pancreatic cancer), pulmonary diseases (e.g., interstitial pneumonitis), developmental disorders (e.g., phocomelia), inflammatory disease, autoimmune diseases, and neurodegenerative disorders.

In addition, Hedgehog-binding cyclopeptibodies of the invention may be used in vitro for research, diagnostic, or clinical purposes such as, for example, for determining the presence and/or expression levels of Hedgehog proteins in a sample or a tissue, for determining the susceptibility of a patient's disease to inhibition of the Hedgehog pathway, for elucidating the role of Hedgehog pathway signaling in a cellular pathway or process, and the like.

The therapeutic benefit of blocking the interaction between the programmed cell death 1 (PD-1) and programmed cell death ligand 1 (PD-L1) for the treatment of cancer is well established (Akinkeye et al. J. Hematol. Oncol. (2019), 12:92), with several therapeutic antibodies targeted against PD-L1 or PD-1 being approved for this application (e.g., atezolizumab, durvalumab, and avelumab). Accordingly, the PD-L1-binding cyclopeptibodies of the invention may be used for the treatment of human cancers that are responsive to the treatment with full-length anti-PD-L1 therapeutic antibodies. In some embodiments, the cancer targeted by a treatment with a PD-L1-binding cyclopeptibody is a blood cancer. In some embodiments, the cancer targeted by a treatment with a PD-L1-binding cyclopeptibody is a solid tumor. In some embodiments, the cancer targeted by a treatment with a PD-L1-binding cyclopeptibody is a colorectal cancer (CRC), castration-resistant prostate cancer (CRPC), non-small-cell lung cancer (NSCLC), or a renal cell carcinoma (RCC).

In addition, the PD-L1-binding cyclopeptibodies of the invention may be used in vitro for research, diagnostic, or clinical purposes such as, for example, for determining the presence and/or expression levels of PD-L1 proteins in a sample or a tissue, for determining the susceptibility of a patient's disease to inhibition of the PD-1/PD-L1 interaction, for labeling PD-L1 expressing cells (e.g., cancer cells), for elucidating the role of PD-L1 mediated signaling in a cellular pathway or process, and the like.

Integrins are cell adhesion and signaling proteins implicated in a wide range of biological functions and pathologies, including cancer, cardiovascular diseases, inflammatory bowel diseases, multiple sclerosis, dry eye disease, and osteoporosis (Slack et al, Nat. Rev. Drug Discov. (2022), 21:60-78). The integrin-binding cyclopeptibodies of the invention may be useful as therapeutic agents, also as part of a combination therapy, for the treatment of pathologies associated with inhibition or activation of integrin function.

In addition, the integrin-binding cyclopeptibodies of the invention may be used in vitro for research, diagnostic, or clinical purposes such as, for example, for determining the presence and/or expression levels of integrin receptor(s) in a sample or a tissue, for determining the susceptibility of a patient's disease to inhibition of integrin function, for labeling integrin-expressing cells (e.g., cancer cells), for elucidating the role of integrin mediated signaling in a cellular pathway or process, and the like.

In some embodiments, the cyclopeptibodies of the invention may be used for improving the delivery of a therapeutic agent to a tissue. In this case, the therapeutic agent is covalently linked to the cyclopeptibody molecule by chemical or enzymatic means according to methods known in the art. In some embodiments, the therapeutic agent is an anticancer agent such as, for example, methotrexate, gemcitabine, vinblastine, and doxorubicin. In some embodiments, the therapeutic agent is an imaging agent such as, for example, a magnetic resonance imaging (MRI) contrast agent or a positron emission tomography (PET) contrast agent.

The terms and expression that are employed herein are used as terms of description and not of limitation, and there is no intention that in the use of such terms and expressions of excluding any equivalents of the features shown and described and portions thereof, but it is recognized that various modifications are possible within the scope of the subject matter claimed herein. Thus, it should be understood that although various embodiments and optional features have been disclosed herein, modification and variation of the concepts herein disclosed may be resorted to those skilled in the art, and that such modifications and variations are considered to be encompassed by the appended claims.

Unless otherwise indicated, the disclosure is not limited to specific molecular structures, substituents, synthetic methods, reaction conditions, or the like, as such may vary. It is to be understood that the embodiments are not limited to particular compositions or biological systems, which can, of course, vary.

A skilled artisan will appreciate that starting materials, biological materials, reagents, synthetic methods, purification methods, analytical methods, assay methods, and biological methods other than those specifically exemplified can be employed in the practice of the methods and compositions disclosed herein. All art-known functional equivalents of any such materials and methods are intended to be included in the methods and compositions disclosed herein.

### 6. EXAMPLES

The following examples are offered by way of illustration and not by way of limitation.

### 6.1 Example 1: Synthesis of Cysteine-Reactive Unnatural Amino Acids

This example demonstrates the preparation of various cysteine-reactive unnatural amino acids, i.e., various Z and Z2 amino acids, which can be used for preparation of macrocyclic peptide molecules according to the general methods illustrated in FIGS. 1, 2, 3, and 4.

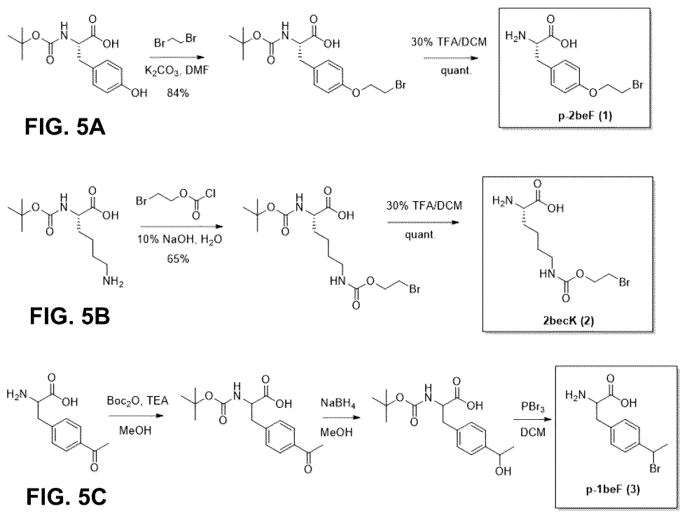

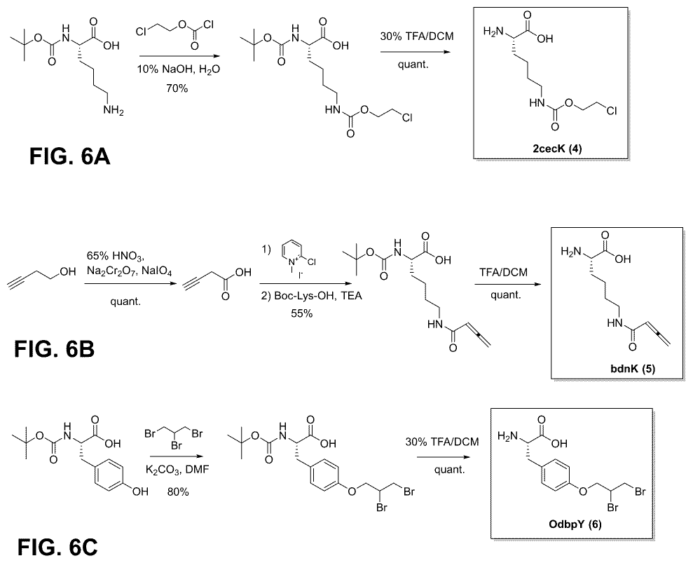

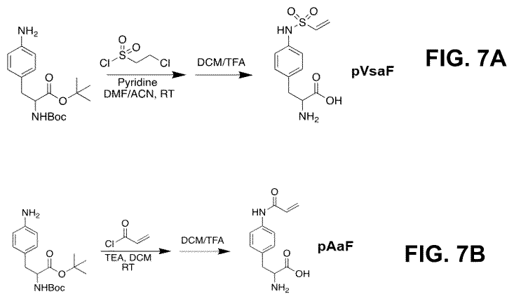

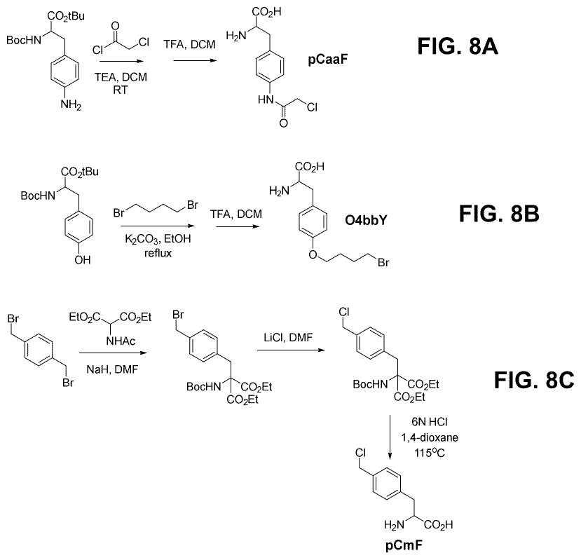

The unnatural amino acid 4-(2-bromoethoxy)-phenylalanine (1, p-2beF) was prepared according to the synthetic route provided in FIG. 5A. The unnatural amino acid N^{ε}-((2-bromoethoxy)carbonyl)-lysine (2, 2-beck) was prepared according to the synthetic route provided in FIG. 5B. The unnatural amino acid 4-(1-bromoethyl)-phenylalanine (3, p-1beF) was prepared according to the synthetic route provided in FIG. 5C. The unnatural amino acid N^{ε}-((2-chloroethoxy)carbonyl)-lysine (4, 2-cecK) was prepared according to the synthetic route provided in FIG. 6A. The unnatural amino acid N^{ε}-(buta-2,3-dienoyl)-lysine (5, bdnK) was prepared according to the synthetic route provided in FIG. 6B. The bifunctional unnatural amino acid O-(2,3-dibromoethyl)-tyrosine (6, OdbpY) was prepared according to the synthetic route provides in FIG. 6C. The unnatural amino acid p-vinylsulfonamido-phenylalanine (pVsaF) was prepared according to the synthetic route provided in FIG. 7A. The unnatural amino acid p-acrylamido-phenylalanine (pAaF) was prepared according to the synthetic route provided in FIG. 7B. The unnatural amino acid p-(2-chloro-acetamido)-phenylalanine (pCaaF) was prepared according to the synthetic route provided in FIG. 8A. The unnatural amino acid O-(4-bromobutyl)-tyrosine (O4bbY) was prepared according to the synthetic route provided in FIG. 8B. The unnatural amino acid p-(chloromethyl)-phenylalanine (pCmF) was prepared according to the synthetic route provided in FIG. 8C. A person skilled in the art would readily recognize that many other cysteine-reactive amino acids of general formula (III), (IV), (VI), or (VII) can be prepared in an analogous manner either through modification of naturally occurring amino acids (e.g., p-2beF, 2-becK, 2-cecK, bdnK, ObdpY, O4bbY) or via synthesis ex novo (e.g., p-1beF, pVSaF, pAaF, pCaaF, pCmF).

### Experimental Details.

Synthesis of 4-(2-bromoethoxy)-phenylalanine (p-2beF) (1). To a reaction flask containing N-tert-butoxycarbonyl-tyrosine (2 g, 7.1 mmol) and potassium carbonate (2.94 g, 21.3 mmol) in dry DMF (20 mL) dibromoethane (1.83 mL, 21.3 mmol) was added dropwise over 20 min. The reaction mixture was stirred at room temperature for 18 h after which the reaction mixture was filtered, diluted with 60 mL of water, acidified with acetic acid to pH 4 and extracted with 2×100 mL of EtOAc. Organic layers were combined and dried over sodium sulfate. The solvent was removed under reduced pressure yielding yellow oil as crude product which was purified by flash column chromatography using 10:9:1 hexane:EtOAc:HOAc acid as solvent system. Fractions of interest were combined and solvent removed under reduced pressure yielding N-Boc-4-(2-bromoethoxy)-phenylalanine as an off-white powder (2.3 g, 84%). ¹H NMR (400 MHZ, CD₃OD) δ 1.39 (s, 9H), 2.8-3.05 (m, 2H), 3.3 (t, 2H), 3.51 (t, 2H), 4.37 (t, 2H), 6.69 (d, 2H), 7.02 (d, 2H); ¹³C NMR (125 MHz, CD₃OD) δ 28.73, 29.49, 37.92, 56.82, 65.77, 80.69, 116.27, 128.84, 131.32, 157.39, 157.77, 173.414. MS (ESI) calculated for C₁₄H₁₉NO₅ [M]⁺: m/z 387.07, found 387.17. Purified N-Boc-4-(2-bromoethoxy)-phenylalanine was treated with 20 mL of 30% TFA/DCM to remove the N-terminal protection. Upon completed reaction (determined by TLC), the solvent was removed under reduced pressure, crude residue dissolved 2× in 10 mL of HOAc followed by solvent evaporation yielding the final product 1 as an off-white solid in quantitative yield (1.7 g). ¹H NMR (400 MHZ, CD₃OD) δ 3.05-3.25 (m, 2H), 3.58 (t, 2H), 4.28 (t, 1H), 4.51 (t, 2H), 6.77 (d, 2H), 7.09 (d, 2H); ¹³C NMR (125 MHz, CD₃OD) δ 29.1, 36.9, 55.35, 66.92, 116.92, 125.54, 131.59, 158.41, 169.93. MS (ESI) calculated for C₁₁H₁₄BrNO₃ [M+H]⁺: m/z 288.02, found 288.51.

Synthesis of N^{ε}-((2-bromoethoxy)carbonyl)-lysine (2becK) (2). To a solution of Na-tert-butoxycarbonyl-lysine (1 g, 4.06 mmol) and NaOH (162.4 mg, 4.06 mmol, 1 eq) dissolved in 20 mL of water 2-bromoethylchloroformate (0.435 mL, 4.06 mmol, 1 eq) and, separately, an additional equivalent of NaOH were added simultaneously dropwise over 30 min. The reaction mixture was stirred at room temperature for 18 h. Upon acidification with HOAc, the aqueous phase was extracted with EtOAc (3×80 mL). The combined organic phases were dried over sodium sulfate, solvent was removed under reduced pressure yielding yellow oil as crude product which was purified by flash column chromatography using 10:9:1 hexane:EtOAc:HOAc as solvent system. Fractions of interest were combined and solvent removed under reduced pressure yielding N-Boc-N^{ε}-((2-bromoethoxy)carbonyl)-lysine as an off-white powder (1.1 g, 68%). ¹H NMR (400 MHZ, CD₃OD) δ 1.43 (s, 9H), 1.5 (m, 2H), 1.65 (m, 2H), 1.79 (m, 2H), 3.09 (t, 2H), 3.54 (t, 2H), 4.05 (t, 1H), 4.29 (t, 2H); ¹³C NMR (125 MHz, CD₃OD) δ 24.09, 28.78, 30.39, 30.47, 32.434, 41.44, 54.82, 65.51, 80.51, 158.15, 158.44, 176.24; MS (ESI) calculated for C₁₄H₁₉NO₅ [M+H]⁺: m/z 397.1, found 397.47. Purified N-Boc-Ng-((2-bromoethoxy)carbonyl)-lysine was treated with 20 mL of 30% TFA/DCM to remove the N-terminal protection. Upon completed reaction (determined by TLC), the solvent was removed under reduced pressure, crude residue dissolved 2× in 10 mL of acetic acid followed by solvent evaporation yielding the final product 2 as an off-white solid in quantitative yield (0.82 g). ¹H NMR (400 MHZ, CD₃OD) δ 1.45 (m, 2H), 1.64 (m, 2H), 1.76 (m, 2H), 2.95 (t, 2H), 3.6 (t, 2H), 3.85 (t, 1H), 4.22 (t, 2H); ¹³C NMR (100 MHz, CD₃OD) δ 20.74, 23.16, 30.36, 31.16, 41.21, 53.86, 65.54, 158.52, 175.21; MS (ESI) calculated for C₁₁H₁₄BrNO₃ [M+H]⁺: m/z 297.04, found 297.7.

Synthesis of 4-(1-bromoethyl)-phenylalanine (p-1beF) (3). Solution of 4-acetylphenylalanine (0.5 g, 2.415 mmol), prepared as reported previously (Frost et al. 2013), in methanol was placed in an ice bath followed by addition of triethylamine (0.51 mL, 3.63 mmol, 1.5 eq) and dropwise addition of di-tert-butyl dicarbonate (0.665 mL, 2.9 mmol, 1.2 eq) over 30 min. The reaction was left at room temperature for additional 3 h after which the solvent was removed in vacuo. The residue was redissolved in EtOAc and extracted with acidified water (pH 4). Organic phase was dried over sodium sulfate, solvent removed under reduced pressure and the crude yellow oil purified using flash column chromatography with 10:9:1 hexane:EtOAc:HOAc as solvent system. Fractions of interest were combined yielding N-Boc-4-acetylphenylalanine as a yellow powder (0.665 g, 90%) which was dissolved in MeOH, placed in an ice bath and treated with NaBH₄ (0.164 g, 4.34 mmol, 2 eq) for 3 h. Following aqueous workup, the crude product was dissolved in DCM, placed in an ice bath and PBr₃ (1 M solution in DCM) was added in portions (5.2 mL, 5.2 mmol, 2.4 eq) over 2 h. The reaction was warmed to reach room temperature and left stirring overnight. After workup, the aqueous layer was lyophilized and used as crude product 3 (0.382 g, 65%). ¹H NMR (400 MHZ, CD₃OD) δ 1.99 (d, 3H), 2.8-3.2 (m, 2H), 4.31 (t, 1H), 4.78 (q, 1H), 7.18 (d, 2H), 7.27 (d, 2H); MS (ESI) calculated for C₁₁H₁₄BrNO₂ [M+H]⁺: m/z 272.03, found 272.53.

Synthesis of N^{ε}-((2-chloroethoxy)carbonyl)-lysine (2-cecK) (4). To a solution of Na-tert-butoxycarbonyl-lysine 1 (1 g, 4.06 mmol) and NaOH (162.4 mg, 4.06 mmol, 1 eq) dissolved in 20 mL of water 2-chloroethylchloroformate (0.419 mL, 4.06 mmol, 1 eq) and, separately, an additional equivalent of NaOH were added simultaneously dropwise over 30 min. The reaction mixture was stirred at room temperature for 10-12 h. Upon acidification with HOAc, the aqueous phase was extracted with EtOAc (3×80 mL). The combined organic phases were dried over sodium sulfate, solvent was removed under reduced pressure yielding yellow oil as crude product which was purified by flash column chromatography using 10:9:1 hexane:EtOAc:HOAc as solvent system. Fractions of interest were combined and solvent removed under reduced pressure yielding off-white powder as product (1.04 g, 75%). Purified product was treated with 20 mL of 30% TFA/DCM to remove the SN-terminal Boc-protection. Upon completed reaction (determined by TLC), the solvent was removed under reduced pressure, yielding the final product 4 as off-white solid in quantitative yield (0.75 g). ¹H NMR (400 MHZ, CD₃OD) δ 1.45 (m, 2H), 1.64 (m, 2H), 1.76 (m, 2H), 2.95 (t, 2H), 3.6 (t, 2H), 3.85 (t, 1H), 4.22 (t, 2H).

Synthesis of N^{ε}-(buta-2,3-dienoyl)-lysine (bdnK) (5). 3-butynoic acid was prepared by oxidation of 3-butyn-1-ol. About 20 mL of water was added to a 150 mL single neck RBF followed by 65% HNO₃ (45 μL, 0.66 mmol, 0.05 eq), Na₂Cr₂O₇ (40 mg, 0.132 mmol, 0.01 eq) and NaIO₄ (6.22 g, 29 mmol, 2.2 eq) and stirred vigorously on an ice bath. After 15 min 1 mL of 3-butyn-1-ol (1 eq, 13.2 mmol) dissolved in 5 mL of cold water was added dropwise over 30 min. The reaction was left stirring overnight followed by product extraction with diethyl ether. Solvent was evaporated to yield off-white/yellow solid (g, %). ¹H NMR (400 MHZ, CDCl3) δ 3.35 (d, 2H), 2.22 (t, 1H). 3-butynoic acid (0.436 g, 5.2 mmol, 1 eq) was dissolved in dry DCM and 1.5 eq of 2-chloro-1-methylpyridinium iodide was added (2.2 g). The reaction was stirred for 1 h at room temperature followed by dropwise addition of N^{ε}-tert-butoxycarbonyl-lysine (1.4 g, 5.72 mmol, 1.1 eq) and triethylamine (1.2 mL, 7.8 mmol, 1.5 eq). The reaction was monitored by TLC and upon completion (4-5 h) extracted with water. Organic layer was evaporated and the crude product was purified using flash column chromatography with 10:9:1 hexane:EtOAc:HOAc as solvent system. Fractions containing the desired product were pooled together and the solvent was removed under reduced pressure giving the desired product in 55% yield. ¹H NMR (400 MHZ, CD₃OD) δ 1.4 (s, 9H), 1.5 (m, 2H), 1.62 (m, 2H), 1.81 (m, 2H), 3.13 (t, 2H), 4.51 (m, 3H), 5.8 (m, 1H). The final Boc-deprotection was achieved using 20 mL of 30% TFA/DCM for 30 min followed by solvent removal resulting in product 5 (g). ¹H NMR (400 MHZ, CD₃OD) δ 1.48 (m, 2H), 1.63 (m, 2H), 1.82 (m, 2H), 3.12 (t, 2H), 4.21 (t, 1H), 4.51 (d, 2H), 5.8 (m, 1H).

Synthesis of O-(2,3-dibromoethyl)-tyrosine (OdbpY) (6). To a reaction flask containing Na-tert-butoxycarbonyl-tyrosine (2 g, 7.1 mmol) and potassium carbonate (2.94 g, 21.3 mmol, 2 eq) in dry DMF (20 mL) 1,2,3-tribromopropane (0.915 mL, 7.82 mmol, 1.1 eq) was added dropwise over 20 min. The reaction mixture was stirred at room temperature for 8 h after which the reaction mixture was filtered, diluted with 60 mL of water, acidified with acetic acid to pH 4 and extracted with 2×100 mL of EtOAc. Organic layers were combined and dried over sodium sulfate. The solvent was removed under reduced pressure yielding yellow oil as crude product which was purified by flash column chromatography using 10:9:1 hexane:EtOAc:HOAc acid as solvent system. Fractions of interest were combined and solvent removed under reduced pressure yielding off-white powder as product (g, %). ¹H NMR (400 MHz, CD₃OD) δ 1.41 (s, 9H), 2.81-3.07 (m, 2H), 3.6-3.81 (m, 2H), 4.21-4.43 (m, 3H), 4.61-4.72 (m, 1H), 6.71 (d, 2H), 7.04 (d, 2H). Purified product was treated with 20 mL of 30% TFA/DCM to remove the N-terminal protection. Upon completed reaction (determined by TLC), the solvent was removed under reduced pressure yielding the final product 6 as an off-white solid in quantitative yield (g). ¹H NMR (400 MHZ, CD₃OD) δ 2.81-3.07 (m, 2H), 3.6-3.81 (m, 2H), 4.12 (t, 1H), 4.21-4.43 (m, 2H), 4.61-4.72 (m, 1H), 6.71 (d, 2H), 7.04 (d, 2H).

Synthesis of p-vinylsulfonamido-phenylalanine (pvSaF). N-Boc-O-t-Bu-4-aminophenylalanine (0.7 g, 2.1 mmol) was dissolved in a 1:1 mixture of DMF/ACN (7 mL total). To the reaction flask was added pyridine (0.5 mL, 6.3 mmol, 3.0 equiv), followed by the dropwise addition of 2-chloroethanesulfonyl chloride (0.3 mL, 3.1 mmol, 1.5 equiv). The reaction was allowed to stir at RT for 3 h and was quenched with 5% HCl (20 mL). The reaction mixture was transferred to a separatory funnel and extracted with EtOAc (20 mL) and the phases were separated. The aqueous phase was adjusted to neutral pH and was extracted with 2×20 mL of EtOAc. Organic layers were pooled, washed with brine and dried over Na₂SO₄. Solvent was removed via rotary evaporation to yield a crude orangish-yellow solid. Crude was adsorbed onto silica gel and purified via flash chromatography (10:3 Hex:EtOAc) to afford N-Boc-O-t-Bu-4-vinylsulfonamide phenylalanine as an orangish-yellow oil. The purified product was deprotected in a 1:1 mixture of DCM:TFA (10 mL) for 5 h at RT. Volatiles were removed by rotary evaporation. The reaction mixture was taken up in Et₂O (20 mL) and removed via rotary evaporation. This process was repeated 5× to remove residual TFA. The deprotected product was taken up in 10 mL of 50 mM HCl, flash frozen, and lyophilized to afford an orange solid (0.14 g, 25% yield).

Synthesis of p-acrylamido-phenylalanine (pAaF): N-Boc-O-t-Bu-4-aminophenylalanine (0.6 g, 1.8 mmol) was dissolved in dichloromethane (6 mL). To the reaction flask was added triethylamine (0.7 mL, 5.3 mmol, 3.0 equiv), followed by the dropwise addition of acryloyl chloride (0.2 mL, 2.7 mmol, 1.5 equiv). The reaction was allowed to stir at RT for 3 h and solvent was removed via rotary evaporation. The reaction mixture was taken up in EtOAc (20 mL) and transferred to a separatory funnel and the reaction was extracted with ddH₂O (30 mL). The aqueous phase was extracted with 2×20 mL of EtOAc. Organic layers were pooled, washed with brine and dried over Na₂SO₄. Solvent was removed via rotary evaporation to yield a crude yellow solid. Crude was adsorbed onto silica gel and purified via flash chromatography (10:3 Hex:EtOAc) to afford N-Boc-O-t-Bu-4-acrylamide phenylalanine as a yellow oil. The purified product was deprotected in a 1:1 mixture of DCM:TFA (10 mL) for 5 h at r.t. Volatiles were removed by rotary evaporation. The reaction mixture was taken up in Et₂O (20 mL) and removed via rotary evaporation. This process was repeated 5× to remove residual TFA. The deprotected product was taken up in 10 mL of 50 mM HCl, flash frozen, and lyophilized to afford a brown solid (0.11 g, 27% yield)

Synthesis of p-(2-chloro-acetamido)-phenylalanine (pcAaF): N-Boc-O-t-Bu-4-aminophenylalanine (0.7 g, 2.1 mmol) was dissolved in dichloromethane (7 mL). To the reaction flask was added triethylamine (0.8 mL, 6.2 mmol, 3.0 equiv), followed by the dropwise addition of chloroacetyl chloride (0.3 mL, 3.1 mmol, 1.5 equiv). The reaction was allowed to stir at RT for 3 h and solvent was removed via rotary evaporation. The reaction mixture was taken up in EtOAc (20 mL) and transferred to a separatory funnel and the reaction was extracted with ddH₂O (30 mL). The aqueous phase was extracted with 2×20 mL of EtOAc. Organic layers were pooled, washed with brine and dried over Na₂SO₄. Solvent was removed via rotary evaporation to yield a crude yellow solid. Crude was adsorbed onto silica gel and purified via flash chromatography (10:3 Hex:EtOAc) to afford N-Boc-O-t-Bu-4-chloroacetamide phenylalanine as a yellow oil. The purified product was deprotected in a 1:1 mixture of DCM:TFA (10 mL) for 5 h at RT. Volatiles were removed by rotary evaporation. The reaction mixture was taken up in Et₂O (20 mL) and removed via rotary evaporation. This process was repeated 5× to remove residual TFA. The deprotected product was taken up in 10 mL of 50 mM HCl, flash frozen, and lyophilized to afford a yellow solid (0.49 g, 92% yield).

Synthesis of O-4-bromobutyl tyrosine (O4bbY). N-Boc-O-t-Bu-tyrosine (1.0 g, 2.9 mmol) was dissolved in EtOH (10 mL). To the reaction flask was added K₂CO₃ (1.2 g, 8.8 mmol, 3.0 equiv) and 1,4-dibromobutane (3.5 mL, 29.6 mmol; 10 equiv). The reaction flask was fitted with a reflux condenser, placed in an oil bath, and allowed to stir at reflux overnight and solvent was removed via rotary evaporation. The reaction mixture was taken up in EtOAc (30 mL) and transferred to a separatory funnel and the reaction was extracted with ddH₂O (50 mL). The aqueous phase was extracted with 2×20 mL of EtOAc. Organic layers were pooled, washed with brine and dried over Na₂SO₄. Crude was purified via flash chromatography (10:1 Hex:EtOAc) to afford N-Boc-O-t-Bu-O-4-bromobutyltyrosine as a white solid. The purified product was deprotected in a 1:1 mixture of DCM:TFA (20 mL) for 5 h at RT. Volatiles were removed by rotary evaporation. The reaction mixture was taken up in Et₂O (30 mL) and removed via rotary evaporation. This process was repeated 5× to remove residual TFA. The deprotected product was taken up in 10 mL of 50 mM HCl, flash frozen, and lyophilized to afford a white solid (0.8 g, 89% yield).

Synthesis of p-chloromethyl phenylalanine (pCmF). Diethyl acetamidomalonate (435 mg, 2 mmol) was dissolved in 10 mL dry DMF and the solution was cooled down in an ice bath under inert conditions. NaH (60% dispersed in mineral oil, 120 mg, 3 mmol) was added and the mixture was stirred for 10 minutes. This was followed by the addition of 1,4-bis-(bromomethyl)-benzene (792 mg, 3 mmol) and the reaction mixture was stirred overnight at room temperature. Progress of the reaction was monitored by TLC. Upon completion, DMF was removed in vacuo and the crude product was dissolved in ethyl acetate (100 ml), washed with water (50 mL, 2X), brine (50 mL) and dried over sodium sulfate. Flash chromatography with a gradient of 100% DCM to 15% EtOAc/DCM afforded the desired bromomethylbenzene derivative in 55% yield. The product from this step (441 mg, 1.1 mmol) was then dissolved in 5 mL DMF. LiCl (140 mg, 3.3 mmol) was added to this under ice-cold conditions and the mixture was stirred for 2 hours at room temperature. Upon completion, DMF was evaporated, and the product was dissolved in EtOAc (100 mL). This mixture was washed with water (50 mL, 3×), brine (50 mL) and dried over sodium sulfate and concentrated in vacuo to afford the desired bromomethylbenzene derivative in 91% yield. This product was then dissolved in 15 mL of 6 N HCl in 1,4-Dioxane and refluxed at 114° C. for 4 hours. The reaction mixture was then dried in vacuo, washed with Et₂O, and was dissolved in 10 ml water and lyophilized to afford pCmF in quantitative yield.

### 6.2 Example 2: Polynucleotides for Expression of Precursor Polypeptides

This example demonstrates procedures for the construction of polynucleotide molecules for the expression of precursor polypeptides of the type (I), (II), or (V) according to the methods described herein.

Plasmid-based vectors were prepared that encode for cyclopeptibody molecules in different formats (Table 1) according to the macrocyclization methods schematically described in FIGS. 1, 2, 3, and 4. Specifically, a series of constructs (e.g. Entries 1, 3, 5, 13-16, Table 1) were prepared for the expression of precursor polypeptides of general formula (I), in which (i) the N-terminal tail, (AA)ₘ, comprises a FLAG tag; (ii) the target peptide sequence, (AA) n, comprises a peptide sequence of variable sequence and length; and (iii) the C-terminal tail, (AA)ₚ, comprises a spacer sequence (Gln)₆, followed by a human IgG1 Fc domain, which is C-terminally fused to a polyhistidine affinity tag. Other constructs were prepared that have a similar structure but without the C-terminal polyhistidine affinity tag (e.g., Entries 2 and 4, Table 1). Other constructs were prepared that have a similar structure to the latter but contain a different spacer sequence (e.g., Entries 7 and 10, Table 1). Other constructs were prepared, wherein (i) the N-terminal tail, (AA)ₘ, comprises a human IgG1 Fc domain followed by a spacer sequence (Gln)6; (ii) the target peptide sequence, (AA) n, comprises a five-amino acid long peptide sequence; and (iii) the C-terminal tail, (AA)ₚ, comprises three-amino acid long peptide sequence spacer sequence (e.g., Entries 12, Table 1). As it can be appreciated by a person skilled in the art, polynucleotide molecules for the expression of many other and different structures of cyclopeptibody molecules, including cyclopeptibody molecules containing multiple copies of the same or different cyclic peptide can be prepared using the general methods described in FIGS. 1 and 2.

### Experimental Details.

Cloning and plasmid construction. The plasmid vector pET22b (+) (Novagen) was used as cloning vector to prepare the plasmids for the expression of the precursor polypeptides described in Table 1. Briefly, synthetic oligonucleotides (Integrated DNA Technologies) were used for PCR amplification of a gene encoding for N-terminal peptide and peptide target sequence and the resulting PCR product was fused to a gene encoding for the Fc domain of human IgG1 via SOE PCR. For the constructs containing a C-terminal HisTag, the resulting gene was digested with Nde I and Xho I and cloned into pET22b (+) to provide the plasmids for the expression of the precursor polypeptides in his-tagged form. For the constructs lacking a C-terminal HisTag, a stop codon was introduced at the 3′-end of the fusion gene. The gene encoding for the human IgG1 was cloned from a plasmid encoding for the full-length anti-HER2 antibody transtuzumab. In correspondence of the position encoding for the non-canonical amino acid Z, an amber stop was introduced in the corresponding gene to enable the genetic incorporation of the desired Z amino acid via amber stop codon suppression. The sequences of the plasmid constructs were confirmed by DNA sequencing.

### 6.3 Example 3: Identification of tRNA/Aminoacyl-tRNA Synthetase Pairs for Incorporation of Cysteine-Reactive Amino Acids

This example illustrates how a suitable tRNA/aminoacyl-tRNA synthetase pair can be identified for the purpose of incorporating a desired cysteine-reactive, non-canonical amino acid into a precursor polypeptide of general formula (I), (II), or (V) according to the methods disclosed herein. In particular, this example describes the identification of tRNA/aminoacyl-tRNA synthetase pairs for the incorporation of the unnatural amino acid p-(chloromethyl)-phenylalanine (pCmF), which was synthesized as described in Example 1. Furthermore, this Example demonstrates an exemplary procedure for assessing the efficiency of said non-canonical amino acid to produce a cyclic peptide via proximity-induced alkylation of a proximal cysteine residue, as useful for the preparation of the cyclopeptibody of the invention.

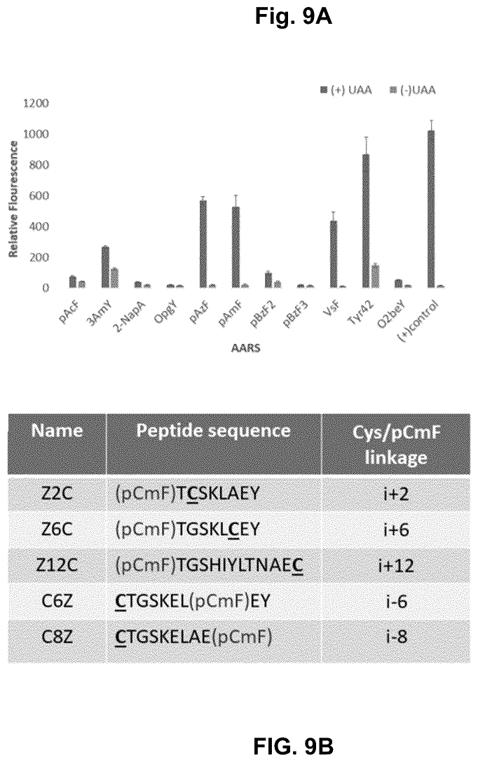

A high-throughput fluorescence-based screen was applied to identify viable (RNA/aminoacyl-tRNA synthetase (AARS) pairs for the ribosomal incorporation of the unnatural amino acid pCmF, in response to an amber stop codon. In this assay, E. coli cells are co-transformed with two plasmids with compatible origins of replications and selection markers; one plasmid directs the expression of the tRNA/AARS pair to be tested, whereas the second plasmid contains a gene encoding for a variant of Yellow Fluorescence Protein (YFP), in which an amber stop codon (TAG) is introduced at the second position of the polypeptide sequence following the initial Met residue (called YFP (TAG)). The ability of the tRNA/AARS pair to suppress the amber stop codon with the unnatural amino acid of interest can be thus determined and quantified based on the relative expression of YFP as determined by fluorescence. Using this assay, a panel of engineered aminoacyl-tRNA synthetase (AARS) variants derived from M. jannaschii tyrosyl-tRNA synthetase (SEQ ID NO:77), in combination with the cognate amber stop codon suppressor tRNA (i.e., MjtRNA_{CUA}^{Tyr} (SEQ ID NO:101)) were tested for their ability to incorporate the target amino acids p-2beF, 2beck, p-1beF, 2cecK, bdnK, or OdbpY into the reporter YFP (TAG) protein. In this experiment, the panel of AARS enzymes comprised a series of known engineered MjTyrRS variants, including Mj-pAcF-RS (SEQ ID NO:81), Mj-3AmY-RS (SEQ ID NO: 82), Mj-2NapA-RS (SEQ ID NO: 83), Mj-OpgY-RS (SEQ ID NO: 84), Mj-Opg Y2-RS (SEQ ID NO: 85), Mj-pAzF-RS (SEQ ID NO: 86), Mj-pAmF-RS (SEQ ID NO: 87), Mj-pBzF2-RS (SEQ ID NO: 88), Mj-TyrRS42 (SEQ ID NO: 190), Mj-VsF-RS (SEQ ID NO: 191), among others. As illustrated by the data in FIG. 9A, various AARS/tRNA pairs were determined to enable the genetic incorporation of pCmF into the reporter protein, from which Mj-Tyr42RS/MjtRNA_{CUA}^{Tyr} was selected as the most efficient one. Control experiments with no unnatural amino acid added to the culture medium show no or negligible expression of the reporter YFP protein, evidencing the discriminating selectivity of these AARS/tRNA pairs for the desired unnatural amino acid over the pool of natural amino acids (this property is referred here as “orthogonal reactivity” or simply “orthogonality” of the AARS/tRNA).

pCmF and Mj-Tyr42RS/MjtRNA_{CUA}^{Tyr} were then utilized to produce a series of constructs in which target peptide sequences containing pCmF and a Cys residue at varying distances and orientations are fused to a his-tagged chitin-binding domain protein (FIG. 9B). Upon expression of these polypeptide constructs in E. coli and purification via Ni-affinity chromatography, efficient cyclization of the pCmF/Cys pair (>90-95% yield) was established via MALDI-TOF mass spectrometry, which established the functionality of pCmF for the preparation of cyclopeptibodies. Using a similar approach, the functionality of other non-canonical amino acids of general structure Z such as, for example, pVsaF, pCaaF, and pAaF, is known (e.g., Iannuzzelli & Fasan, Chem. Sci., 2020, 11, 6202).

These results provide an exemplary demonstration of viable procedures that can be used to identify suitable non-canonical amino acids of general structure Z, as well as suitable AARS/tRNA pairs for the ribosomal incorporation of these non-canonical amino acids into a polypeptide for the purpose of producing cyclopeptibody molecules according to methods disclosed herein and as illustrated in the following Examples.

### Experimental Details.

YFP expression assay. Competent BL21 (DE3) E. coli were cotransformed with a pEVOL-based plasmid (Smith et al. 2011) for the expression of the desired AARS/tRNA pair and a pET22-YFP (TAG) plasmid for the expression of the reporter YFP protein. After overnight growth at 37° C. in LB medium supplemented with chloramphenicol (25 μg/mL) and ampicillin (50 μg/mL), cell cultures were used to inoculate 96-well plates containing 0.9 mL of minimal (M9) media (25 μg/mL chloramphenicol, 50 g/mL ampicillin, 1% glycerol) per well. At OD₆₀₀=0.6, protein expression was induced with 0.05% L-arabinose and 1 mM IPTG. Test wells were supplemented with the desired unnatural amino acid (e.g., 4-(chloromethyl)-phenylalanine (pCmF) at 2 to 5 mM, whereas no amino acid was added to the negative control wells. Cultures were grown overnight at 27° C. and then diluted 1:100 with phosphate buffer (50 mM KPi (pH 7.5), 150 mM NaCl) into microtiter plates. Fluorescence intensity was measured using a Tecan Infinite 1000 multi-well plate reader (λ_{exc}: 514 nm; λₑₘ: 527 nm).

### 6.4 Example 4: Preparation and Isolation of Cyclopeptibodies

This example demonstrates procedures for the expression and purification of cyclopeptibody molecules in a prokaryotic expression system according to the methods described herein. Furthermore, this example demonstrates the preparation of cyclopeptibody molecules from precursor polypeptide of general formula (I) or (II), according to the methods disclosed herein. Furthermore, this example demonstrates the preparation of cyclopeptibody molecules, in which the cyclic peptide comprised within these molecules is cyclized by different types of non-canonical amino acids of general structure Z.

As described in Example 2, plasmid-based vectors for the bacterial expression of cyclopeptibody molecules in different formats (Table 1) were prepared. For the preparation of these constructs, the Fc domain of human IgG1 (SEQ ID NO: 1) was used as source of the Fc region. Two mutations at position 382 and 428 of the Fc domain (E382V/M428I) were introduced to endow the aglycosylated Fc domain with the ability to bind Fc receptors such as FcγRI, FcγRIIa, FcγRIIb, FcγRIIIa (Robinson et al, Nat Commun. (2015) 6:8072). Furthermore, the cysteine residues located at the N-terminal end of the Fc domain (i.e., C226S, C229S), which are involved in forming inter-chain disulfide bridges in the full-length IgG1 antibody, were mutated to serine. These mutations were found to be beneficial for increasing the expression level of the Fc domain, without imparting its activity to dimerize as in the native Fc domain. At its N-terminus (or C-terminus in the case of Table 1, Entry 12), the Fc domain was genetically fused to various target peptide sequences, whose design and choice is described in more detail in the following examples. Finally, in most constructs, a FLAG tag was introduced at the N-terminus of the polypeptide construct to facilitate measurement of the biological activity of the cyclopeptide molecules as described in the following examples. In addition, the N-terminal FLAG tag was found to increase expression yield of the cyclopeptide molecules in E. coli, which was also desirable.

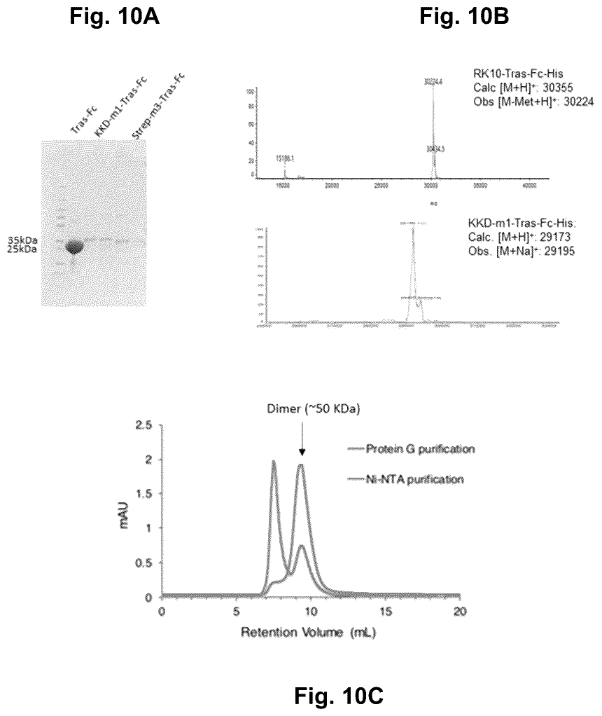

The cyclopeptibody constructs were expressed from pET22-based vectors in BL21 (DE3) cells containing a second, pEVOL-based plasmid for co-expression of the appropriate orthogonal AARS/tRNA pair for incorporation of the desired unnatural amino acid (e.g., O2beY, pCaaF, pCmF; as reported in Table 1) via amber stop codon suppression. After expression, the proteins were and purified via protein G-affinity chromatography or via Ni-affinity chromatography for the His-tagged constructs. As shown by the representative data in FIG. 10, the purity of these constructs was confirmed by SDS-PAGE gel electrophoresis (FIG. 10A), whereas the incorporation of non-canonical amino acids as well as the occurrence of the ncAA-mediated cyclization reaction to form the cyclopeptibody molecule was confirmed by MALDI-TOF mass spectrometry (FIG. 10B). We further established that the cyclopeptibodies purified via Ni-affinity chromatography typically showed higher homogeneity for formation of the dimeric form of the protein, while the constructs purified via protein G affinity chromatography tended to form higher molecular weight oligomers, as determined via gel filtration chromatography (FIG. 10C). Accordingly, the Ni-affinity purification method were chosen as the method of choice for purification of the cyclopeptibody constructs from E. coli.

### Experimental Details.

Protein expression and purification. The protein constructs were expressed using BL21 (DE3) E. coli cells co-transformed with a pET22-based vector for the expression of the precursor polypeptide and a pEVOL-based vector for the expression of the appropriate AARS/tRNA pair (e.g., Mj-pOpgY2-RS/MjtRNA_{CUA}^{Tyr} pair for O2beY; Mj-Tyr42RS—RS/MjtRNA_{CUA}^{Tyr} pair for pCmF; Mj-VsFRS/MjtRNA_{CUA}^{Tyr} pair for pCaaF). Cultures of these cells were grown overnight in LB media (100 mg/L ampicillin; 34 mg/L chloramphenicol) and used to inoculate 0.5 L of minimal (M9) media containing the same concentration of antibiotics, 1% glycerol. At OD₆₀₀=0.6, cell culture was condensed to 0.1 L, L-arabinose (0.06%) and unnatural amino acid (2 mM) were added to the culture media, incubated at 27° C. with shaking for 1 h. IPTG (1 mM) was added to induce protein expression. Cultures were grown for 14 h at 27° C. and then harvested by centrifugation. Cell pellets were resuspended in 50 mM Tris, 300 mM NaCl, 20 mM imidazole buffer (pH 7.5) and cells were lysed by sonication. The cell lysate was loaded on a Ni-NTA affinity column and proteins were eluted with 50 mM Tris, 150 mM NaCl, 300 mM imidazole buffer (pH 7.5). Fractions were combined and concentrated followed by buffer exchange with phosphate-buffered saline pH 7.4. The identity of the isolated proteins was confirmed using MALDI-TOF MS. Purity was analyzed by SDS-PAGE.

### 6.5 Example 5: Design and Generation of a Streptavidin-Binding Cyclopeptibody

This example demonstrates the creation of a functional cyclopeptibody capable of binding the protein streptavidin.

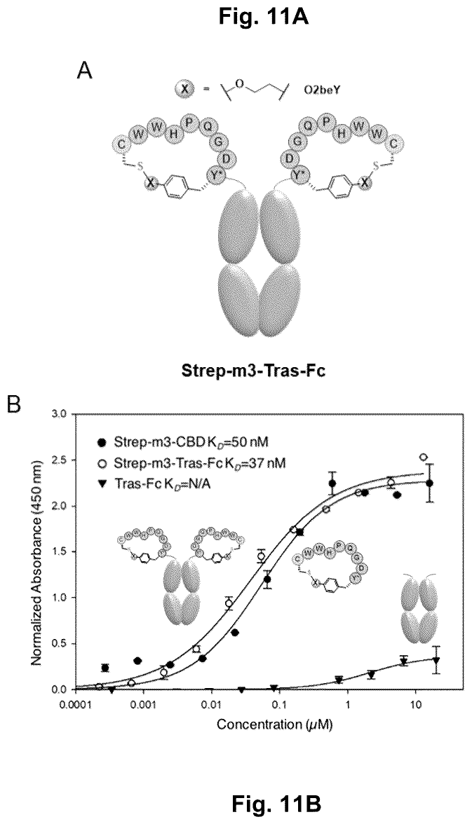

To prepare a cyclopeptibody capable of binding the protein streptavidin, a cyclic streptavidin-binding peptide previously discovered via phage display (Owens et al, ACS Cent Sci. (2020); 6(3):368-381) was genetically fused to the Fc domain of human IgG1 via a spacer (Entry 1, Table 1). The resulting cyclopeptibody (called Strep-m3-Tras-Fc) was expressed, purified, and characterized as described in Example 4. This construct was confirmed to contain the cyclic peptide constrained via O2beY/Cys thioether (i/i-8) linkage, where ‘i’ indicates the position of the non-canonical amino acid O2beY (also refereed to herein as p-2beF). The cyclopeptibody Strep-m3-Tras-Fc was then characterized for its streptavidin binding affinity using a reported in vitro binding assay (Owens et al, ACS Cent Sci. (2020); 6(3):368-381). These experiments showed that cyclopeptibody Strep-m3-Tras-Fc is able to bind streptavidin with high affinity (K_{D}=37 nM), while the Fc domain alone shows no binding (FIG. 11). In addition, the cyclopeptibody Strep-m3-Tras-Fc showed improved (1.3-fold) affinity toward the target protein compared to the same cyclic peptide fused to CBD (K_{D}=50 nM).

### Experimental Details.

Streptavidin Binding Assay. For the streptavidin binding assays, streptavidin-coated plates (Sigma-Aldrich) were used. For the Keap1, Shh and PD-L1 binding assay, target protein was immobilized on microtiter plates by incubating 100 μL of a 4 μM protein solution in PBS buffer overnight at 4° C., followed by decanting the protein solution and blocking with 0.5% bovine serum albumin in PBS for 2 h at room temperature. After washing (3×150 μL of PBS with 0.05% Tween-20), each well was incubated with 100 μL of purified FLAG-tagged cyclopeptibody at varying concentrations for 1 hour at room temperature. After washing three times with wash buffer, each well was incubated with 100 μL of 1:2500 dilution of HRP-conjugate mouse anti-FLAG polyclonal antibody (Sigma-Aldrich) for 1 hour at room temperature. After washing three times with wash buffer, 100 μL of 2.2 mM o-phenylenediamine dihydrochloride, 4.2 mM urea hydrogen peroxide, 100 mM dibasic sodium phosphate, and 50 mM sodium citrate, pH 5.0, were added to each well, followed by measurement of the absorbance at 450 nm after 10-20 min using a Tecan Infinite 1000 plate reader. Equilibrium dissociation constants (Kp) were determined by fitting the dose-response curves to a 1:1 binding isotherm equation via nonlinear regression. using SigmaPlot. Mean values and standard deviations were calculated from experiments performed in triplicate.

### 6.6 Example 6: Design and Generation of a Keap1-Binding Cyclopeptibody

This example demonstrates the creation of a functional cyclopeptibody capable of binding the target protein Kelch-like ECH-associated protein 1 (Keap1). Keap1 is implicated in sequestration and ubiquitination of Nrf2, a transcriptional regulator that promotes the expression of genes that exert a cytoprotective function in response to oxidative stress and reactive (electrophilic) chemicals in human cells.

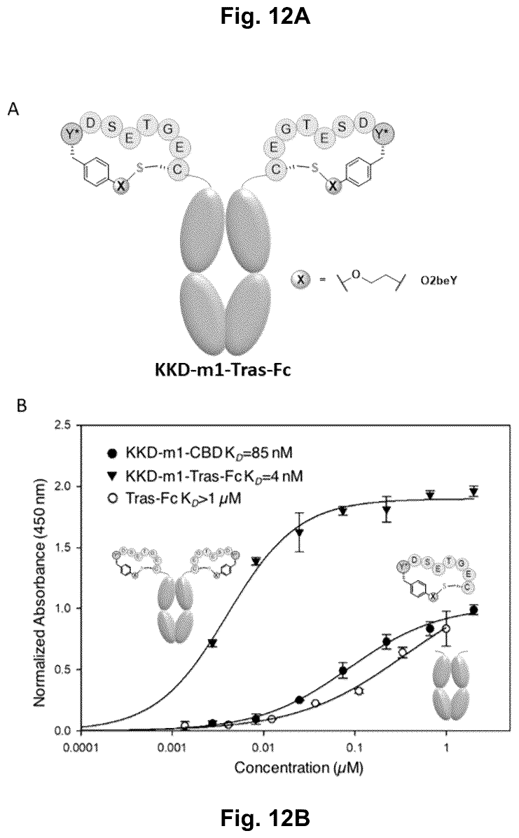

To prepare a cyclopeptibody capable of binding the target protein Keap1, a cyclic Keap1-binding peptide previously discovered via phage display (Owens et al, ACS Cent Sci. (2020); 6(3):368-381) was genetically fused to the Fc domain of human IgG1 via a spacer (Entry 3, Table 1). The resulting cyclopeptibody (called KKD-m1-Tras-Fc) was expressed, purified, and characterized as described in Example 4. This construct was confirmed to contain the cyclic peptide constrained via O2beY/Cys thioether (i/i+7) linkage, where ‘i’ indicates the position of the non-canonical amino acid O2beY (also refereed to herein as p-2beF). The cyclopeptibody KKD-m1-Tras-Fc was then characterized for Keap1 binding affinity using a reported in vitro binding assay (Owens et al, ACS Cent Sci. (2020); 6(3):368-381). These experiments showed that cyclopeptibody KKD-m1-Tras-Fc is able to bind Keap1 with very high affinity (K_{D}=4 nM), while the Fc domain alone shows no significant binding below 100-200 nM (FIG. 12). Importantly, the cyclopeptibody KKD-m1-Tras-Fc showed significantly improved (>20-fold) affinity toward the target protein compared to the same cyclic peptide fused to CBD (K_{D}=85 nM). The large increase in target binding affinity can be attributed, at least in part, to avidity effect due to the bivalency of the cyclopeptibody compared to the cyclic peptide alone. As such, these results demonstrate the functionality and advantageous properties of the cyclopeptibody molecules of the invention.

### Experimental Details.

Keap1 Binding Assay. For the Keap1 binding assay, Keap1 was immobilized on microtiter plates by incubating 100 μL of a 4 μM protein solution in PBS buffer overnight at 4° C., followed by washing (3×150 μL of PBS with 0.5% Tween-20) and blocking with 0.5% bovine serum albumin in PBS for 1.5 h at room temperature. After washing, each well was incubated with 100 μL of purified FLAG-tagged peptide at varying concentrations for 1 hour at room temperature. After washing three times with wash buffer, each well was incubated with 100 μL of 1:2500 dilution of HRP-conjugate mouse anti-FLAG polyclonal antibody (Sigma-Aldrich) for 1 hour at room temperature. After three washing steps with wash buffer, 100 μL of 2.2 mM o-phenylenediamine dihydrochloride, 4.2 mM urea hydrogen peroxide, 100 mM dibasic sodium phosphate, and 50 mM sodium citrate, pH 5.0, were added to each well, followed by measurement of the absorbance at 450 nm after 10-20 min using a Tecan Infinite 1000 plate reader. Equilibrium dissociation constants (K_{D}) were determined by fitting the dose-response curves to a 1:1 binding isotherm equation via nonlinear regression using SigmaPlot. Mean values and standard deviations were calculated from experiments performed in triplicate.

### 6.7 Example 7: Design and Generation of a Hedgehog-Binding Cyclopeptibody

This example demonstrates the creation of a functional cyclopeptibody capable of binding the Sonic Hedgehog (Shh) signaling protein. This example further demonstrates that said cyclopeptibody is capable of binding other analogs of Hedgehog protein, namely Indian (Ihh) and Desert Hedgehog (Dhh). Sonic Hedgehog (Shh) is a key signaling protein implicated in the activation of the Hedgehog pathway. The Hedgehog pathway plays a critical role in controlling embryonic development, and abnormal activation of this pathway has been implicated in various human malignancies, including leukemia and tumors of the prostate, pancreas, and colon (Rubin et al, Nat. Rev. Drug Discovery 2006, 5, 1026-1033; Theunissen et al. Cancer Res. 2009, 69, 6007-6010).

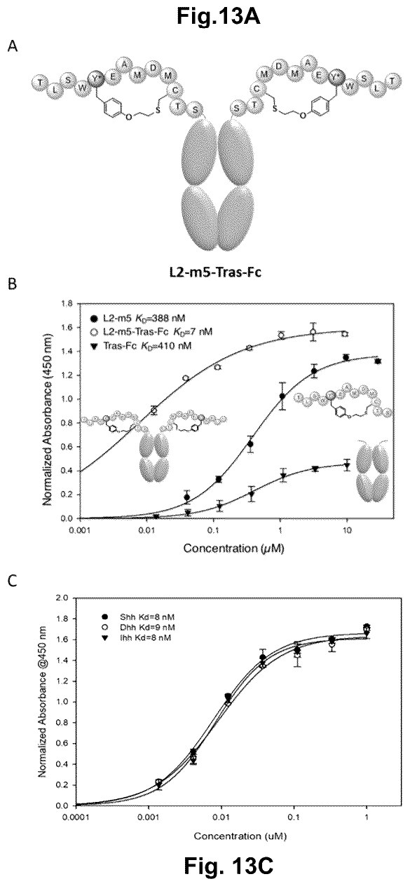

To prepare a Hedgehog-targeting cyclopeptibody, a Shh-binding cyclic peptide previously developed via affinity maturation of a HHIP loop 2 mimic (Owens A E et al., 2017, J. Am. Chem. Soc., 139:12559-12568) was genetically fused to the Fc domain of human IgG1 via a spacer (Entry 5, Table 1). The resulting cyclopeptibody (called L2-m5-Tras-Fc) was expressed, purified, and characterized as described in Example 4. This construct was confirmed to contain the cyclic peptide constrained via O2beY/Cys thioether (i/i+6) linkage, where ‘i’ indicates the position of the non-canonical amino acid O2beY (also referred to herein as p-2beF). The cyclopeptibody L2-m5-Tras-Fc was then characterized for Shh binding affinity using a reported in vitro binding assay (Owens A E et al., 2017, J. Am. Chem. Soc., 139:12559-12568). These experiments showed that cyclopeptibody L2-m5-Tras-Fc is able to bind Shh with very high affinity (K_{D}=7 nM), while the Fc domain alone shows no significant binding below 500 nM (FIG. 13A). Importantly, the cyclopeptibody L2-m5-Tras-Fc showed significantly improved (>55-fold) affinity toward the target protein compared to the same cyclic peptide fused to CBD (K_{D}=388 nM). The large increase in target binding affinity can be attributed, at least in part, to avidity effect due to the bivalency of the cyclopeptibody compared to the cyclic peptide alone. As such, these results further demonstrated the functionality and advantageous properties of the cyclopeptibody molecules of the invention.

The cyclopeptibody L2-m5-Tras-Fc was also tested for its ability to bind other analogs of Hedgehog, namely Desert (Dhh) and Indian (Ihh) Hedgehog, which is desirable for certain therapeutic applications. Indeed, in certain cancers like leukemia aberrant ligand-mediated Hedgehog signaling is mediated by different analogs of Hedgehog. Importantly, these experiments showed that L2-m5-Tras-Fc is capable of binding all analogs of Hedgehog with equally high affinity (K_{D}: 8-10 nM; FIG. 13B). In comparison, the L2-m5 peptide was previously reported to bind Ihh and Dhh with much lower affinity (K_{D}: 250-350 nM, Owens A E et al., 2017, J. Am. Chem. Soc., 139:12559-12568).

### 6.8 Example 8: Design and Generation of Cyclopeptibodies Targeting PD-L1 and the PD-1/PD-L1 Interaction

This example demonstrates the creation of functional cyclopeptibodies capable of binding to PD-L1, a well-established therapeutic target in cancer immunotherapy as described above. This example further demonstrates that said cyclopeptibodies are capable of disrupting the interaction between PD-L1 and PD-1, a key target for blockage of the PD-L1/PD-1 axis in cancer.

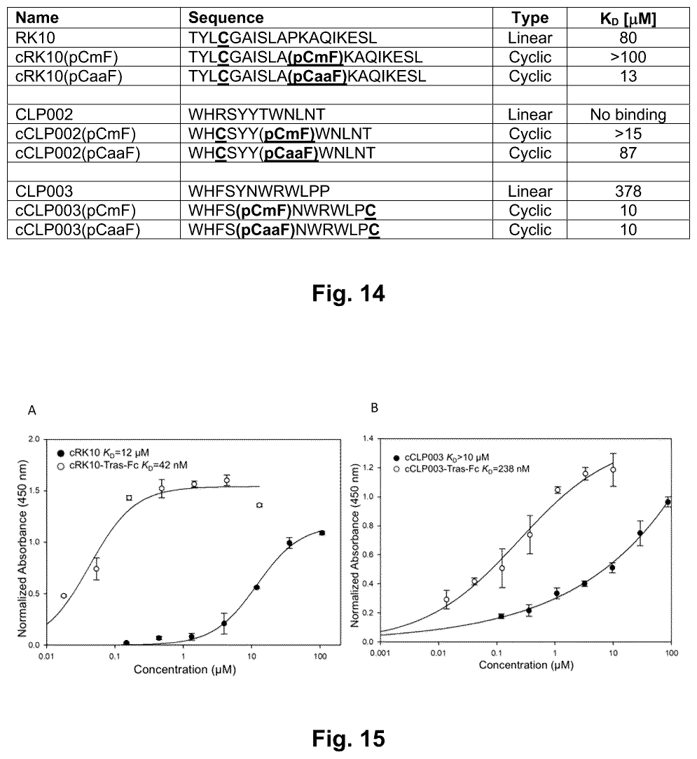

To prepare PD-L1-targeting cyclopeptibody, a series of cyclic peptides cyclized via a cysteine-reactive non-canonical amino acid according to the methods of the invention, were initially designed and tested for their ability to bind PD-L1. Accordingly, three linear peptide sequences known to bind PD-L1, namely RK10 (Caldwell et al., Sci. Rep., 2017, 7, 13682), CLP002 and CLP003 (Liu et al., Immunother. Cancer., 2019, 7, 270), were cyclized using either pCmF or pCaaF located at different position and arrangements (i.e., ncAA/Cys or Cys/ncAA) of these sequences (FIG. 14). The corresponding cyclic peptides as fusions to a CBD affinity tag were recombinantly expressed in E. coli and purified. The CBD tag was enzymatically cleaved and the resulting cyclic peptides were purified by HPLC. The linear peptide counterparts were also produced for comparison purposes. The cyclic and linear peptides were then characterized for their binding affinities to PD-L1 using an in vitro binding assay. These experiments show that the linear peptide RK10 bound to PD-L1 with a Kp of 80 μM, CLP003 with a Kp of 378 μM, whereas the CLP002 showed no detectable binding up to 200 μM. Importantly, the cyclic RK10-based peptide cyclized using pCaaF (called cRK10) showed significantly higher affinity toward the target protein PD-L1 (K_{D}=13 μM vs. 80 μM) compared to both the linear peptide RK10 and the pCmF-cyclized sequence. On the other hand, both cyclic CLP003-based peptides constrained via pCaaF/Cys or pCmF/Cys linkage show a Kp of ˜10 μM for binding to PD-L1, corresponding to a ˜40-fold higher affinity compared to the linear CLP003 peptide. Interestingly, in contrast to the inactive CLP002 linear peptide, both the cyclic CLP002-based peptides show PD-L1 binding activity, with K_{D} in the low micromolar range (FIG. 14).

Based on these results, two cyclopeptibodies containing either the pCaaF-cyclized RK10 sequence (called cRK10-Tras-Fc) or the pCaaF-cyclized CLP003 sequence (called cCLP003-Tras-Fc) were prepared, expressed and isolated as described in Example 4. Upon testing in the in vitro PD-L1 binding assay, cyclopeptibody cRK10-Tras-Fc was determined to bind PD-L1 with a Kp of 40 nM, which corresponds to a 300-fold higher binding affinity compared to the cyclic peptide cRK10 (FIG. 15A). Similarly, the cyclopeptibody cCLP003-Tras-Fc showed submicromolar affinity for PD-L1 with a Kp of ˜250 nM, this corresponding to a 40-fold increase compared to the cyclic peptide alone (FIG. 15B).

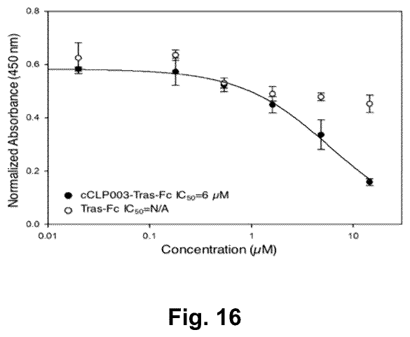

Next, we examined the ability of the cyclopeptibody cCLP003-Tras-Fc to inhibit the PD-1/PD-L1 interaction using a competition assay with recombinant biotinylated PD-1 (Bio-PD-1). Briefly, PD-L1 immobilized on a plate was incubated with Bio-PD-1 and varying concentrations of cCLP003-Tras-Fc or control Fc. The PD-L1-bound PD-1 was then quantified colorimetrically (OD₄₅₀) using horseradish peroxidase (HRP)-conjugated streptavidin. In this assay, cCLP003-Tras-Fc was showed inhibition of PD-1/PD-L1 interaction at low micromolar range (IC₅₀=6 μM), whereas Fc domain alone did not exhibit significant inhibitory activity (FIG. 16). Altogether, these results further demonstrate various embodiments of the invention, such as its use for developing functional cyclopeptibodies capable of binding a target protein of interest, the advantageous features of the cyclopeptibody molecules over the cyclic peptides alone, and their utility as potent agents against a therapeutically relevant protein and protein-protein interaction.

### Experimental Details.

Cloning, Expression and Purification of PD-L1 and Biotinylated PD-1. Genes encoding for PD-L (Genscript) and PD-1 (gift from Tudor Fulga) were subcloned into pET22b (Novagen) respectively, resulting in fusion of poly-histidine sequence to the C-terminus of PD-L1 and PD-1. A synthetic oligonucleotide (Integrated DNA Technologies) was used for the PCR amplification of an Avi tag fused to N-terminal of PD-1. A Cys to Ser mutation was introduced at position 93 of PD-1 to aid expression and folding. PD-L1 and biotinylated PD-1 were expressed in E. coli BL21 (DE3) as inclusion bodies. For PD-L1, the cells were grown at 37° C. in LB medium with ampicillin (50 mg/L) until OD₆₀₀ reached 0.6-1.0, and the protein expression was induced with IPTG (1 mM) and incubated for 4 h at 37° C. For biotinylated PD-1, the plasmid encoding Avi-PD-1 and the pLEMO-BirA plasmid (a gift from Alexander Gabibov) were co-transformed into BL21 (DE3). The cells were grown at 37° C. in LB medium with ampicillin (50 mg/L) until OD₆₀₀ reached 0.6-1.0, and the protein expression was induced with IPTG (1 mM) and BirA expression was induced with 1 mM rhamonse. Biotinylation of PD-1 was catalyzed by BirA with 1 mM Biotin. The culture was incubated for 14 h at 27° C. The cells were harvested by centrifugation (3,400×g for 30 min at 4° C.), re-suspended in lysis buffer (20 mM Tris, pH 8.0, 200 mM NaCl) and lysed by sonication on ice. Inclusion bodies were recovered by centrifugation (21,000×g for 30 min at 4° C.) and solubilized in 8 M urea, 20 mM Tris, pH 8.0, 200 mM NaCl by stirring overnight. After removing undissolved residue by centrifugation (21,000 g for 30 min at 24° C.), solubilized fraction was applied to Ni-NTA chromatography (Invitrogen) according to the manufacturer's instructions and washed with five column volumes of wash buffer (8 M urea, 20 mM Tris, pH 8.0, 200 mM NaCl, 50 mM imidazole). The protein was then eluted with elution buffer (8 M urea, 20 mM Tris, pH 8.0, 200 mM NaCl, 400 mM imidazole). The eluted protein was refolded by dialysis three times against 4 L of 20 mM Tris, pH 8.0, 200 mM NaCl at 4° C., and purified further by gel filtration chromatography using a HiLoad 16/60 Superdex 200 pg column (GE Healthcare Life Sciences). The protein purity was evaluated by reducing and non-reducing SDS-PAGE. Biotinylation of PD-1 was confirmed by MALDI-TOF.

PD-L1 Competition Assay. PD-L1 was immobilized on microtiter plates as described above. followed by decanting the protein solution and blocking with 0.5% bovine serum albumin in PBS for 2 h at room temperature. After washing, each well was incubated with 100 μL of serial dilutions of cCLP003-Tras-Fc-His for 1 hour at room temperature. Biotinylated PD-1 (500 nM) was added and incubated for 1 h at room temperature. After washing, the bound PD-1 was quantified by means of HRP-conjugate Streptavidin and colorimetric assay as described above. The inhibitory constant (IC₅₀) was determined by fitting the dose-response curves to a 4-parameter equation via nonlinear regression using SigmaPlot. Mean values and standard deviations were calculated from experiments performed in triplicate.

### 6.9 Example 9: Application of Cyclopeptibodies as Tumor Imaging and Detection Agents

This example demonstrates how cyclopeptibody molecules of the invention can be applied for detecting a target protein of interest (i.e., PD-L1) on a cancer cell, exemplifying the utility of these compounds as tumor detection and imaging agents.

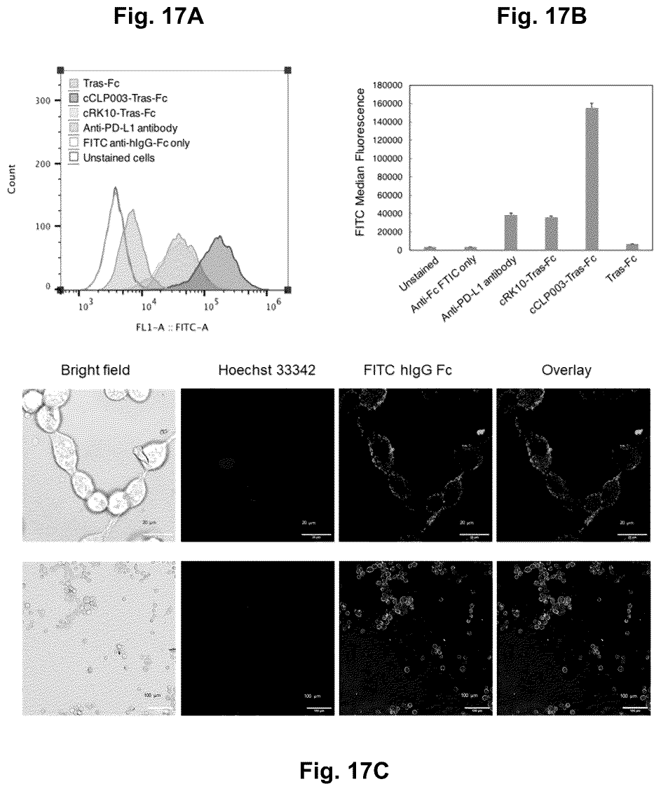

As described in Example 8, the methods disclosed herein were applied to develop two cyclopeptibody molecules capable of binding PD-L1 in vitro, namely cRK10 (pCaaF)-Tras-Fc and cCLP003 (pCaaF)-Tras-Fc. To demonstrate the utility of these compounds for cancer detection/imaging applications, these cyclopeptibodies were incubated with breast cancer cells MDA-MB-231, which are known to express high levels of PD-L1 on their surface (Zheng et al., Oncol. Lett., 2019, 18, 5, 5399-5407). The cells were then incubated with fluorescein-conjugated anti-human IgG Fc antibody and analyzed by flow cytometry. The same experiment was carried out in parallel using commercially available anti-PD-L1 antibody as positive control and various negative controls. These experiments showed positive staining of the MDA-MB-231 cells after treatment with fluorescently labeled anti-PD-L1 antibody (0.005 mg/mL), which confirmed the expression of the PD-L1 receptor on their surface (FIG. 17). Importantly, the same cells treated with cRK10 (pCaaF)-Tras-Fc (0.09 mg/mL) displayed comparable median fluorescence intensity (MFI) to that of the positive control anti-PD-L1 antibody, indicating the ability of the cyclopeptibody to recognize the PD-L1 receptor on the cancer cells. Notably, MDA-MB-231 cells treated with the same amount of the cCLP003 (pCaaF)-Tras-Fc cyclopeptibody (0.09 mg/ml) showed a 4-fold higher MFI than those treated with cRK10-Tras-Fc, indicating that cCLP003 (pCaaF)-Tras-Fc has stronger binding affinity to the PD-L1 receptor on these cells (FIG. 17). Control experiments with the secondary anti-Fc antibody and with the human IgG1 Fc domain-only negligible staining over the untreated cells, indicating that the interaction of the cyclopeptibodies to the cancer cells is specific and dependent upon the presence of the PD-L1 receptor. Altogether, these experiments demonstrate the functionality and efficiency of the PD-L1-targeting cyclopeptibodies for detecting and labeling a biomarker receptor in cancer cells. Several applications for these compounds can be easily envisioned, including detection and/or staining of PD-L1 in vitro and in vivo (e.g., upon conjugation of the cyclopeptibody with a fluorescent dye, IR probe, or PET contrast agent), cell-targeted drug delivery (e.g., upon conjugation of the PD-L1-binding cyclopeptibody with a drug cargo), and use as therapeutic agents in cancer immunotherapy, among others.

### Experimental Details.

Flow Cytometry Analysis. Cell line MDA-MB-231 was grown in culture to reach confluency. When confluent, media was aspirated, cells were washed once with 1×DPBS (no calcium, no magnesium) (Gibco). Adherent cells were detached from the petri dish with 1 mL of accutase (Gibco) pre-warmed to 37° C., followed by incubated for 5 min at 37° C. Cells were spun down using centrifugation. Supernatant was removed and cells were washed three times with staining buffer (1×PBS with 0.5% BSA). Cells were resuspended in Eppendorf tubes in 100 μL PBS at a concentration of 107cells per mL. Cyclopeptibodies or Human anti-PD-L1 antibody (1 μg) (R&D Systems, MAB10355) in staining buffer were then added to the tubes with a concentration of 1.5 μM in 200 μL. Eppendorf tubes were then placed on the platform shaker for gently shaking for 1 hour at room temperature. After 1 hour, cells were spun down using centrifugation, then supernatant was removed. After washing three times, cells were resuspended in 200 μL of Fluorescein (FITC) AffiniPure Goat Anti-Human IgG with working concentration of 30 μg/mL (Jackson ImmunoResearch, 109-095-098), incubated in the dark for 30 min at room temperature. Cells were washed as described above, then resuspended in 400 μL staining buffer. Then cells were analyzed on a BD FACS Accuri C6⁺ using a filter of 533/30. The cells of interest were gated using Forward and Side scatter (FSC/SSC). 10,000 singlet events were collected for each specimen.

### 6.10 Example 10: Design and Generation of Integrin-Targeting Cyclopeptibodies

This example demonstrates the creation of functional cyclopeptibodies capable of binding to an integrin receptor (αᵥβ₃) as the target protein. This example further demonstrates the process of designing and generating an integrin-targeting cyclopeptibody and its application for labeling the integrin receptor (αᵥβ₃) expressed on the surface of a cancer cell.

Integrins are transmembrane receptors that are implicated in mediating cell-cell and cell-extracellular matrix (ECM) adhesion. Integrins are known to bind to peptides containing an RGD motif, which mediates the interaction with a large number of adhesive extracellular matrix, blood, and cell surface proteins. Nearly half of the over 20 known integrin receptor isoforms recognize the RGD motifs. Because of their role in regulating cell migration, growth, differentiation, and apoptosis, integrin receptors are over-expressed on cell surface of several types of human cancers. Accordingly, compounds directed against integrin receptors have been investigated for the therapeutic value for the treatment of cancer as well as other diseases such as thrombosis and osteoporosis.

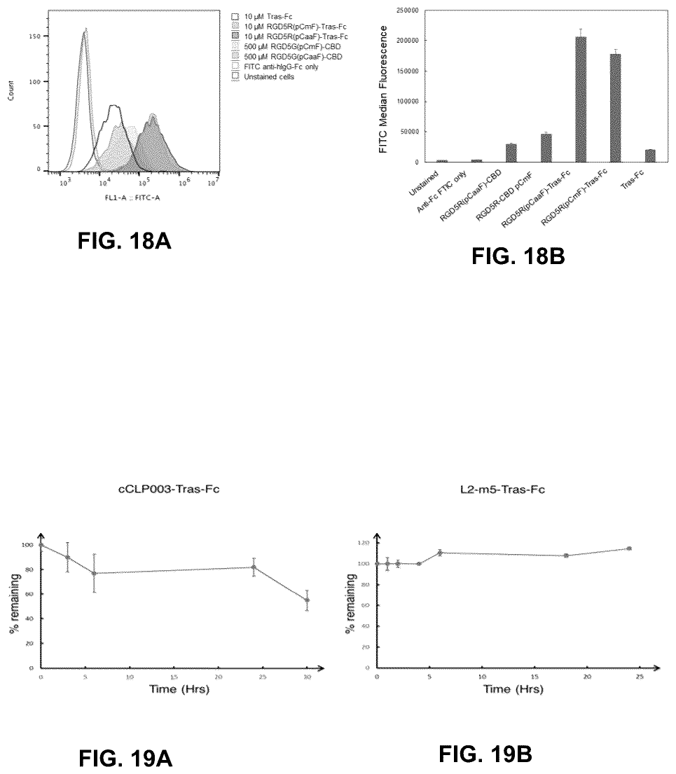

To generate an integrin-targeting cyclopeptibody, we started by identifying a genetically encodable cyclic peptide that is capable of binding to an integrin receptor, using the cancer-related integrin αᵥβ₃ as the target. Accordingly, we designed a panel of cyclic peptides whose sequence incorporate an RGD motif and which are cyclized through a O2beY/Cys, pCaaF/Cys, or pCmF/Cys pair via different connectivity (i.e., ncAA/Cys and Cys/ncAA) (Table 2 (SEQ ID Nos: 241-258) and FIG. 18A). This choice was motivated by knowledge of the effect of the type of ncAA and orientation of the ncAA/Cys on modulating the functional properties of these thioether-constrained peptides, as determined in our prior studies (Iannuzzelli & Fasan, Chem. Sci., 2020, 11, 6202). The cyclic peptides were produced recombinantly with a N-terminal FLAG tag and C-terminal chitin-binding domain (CBD) tag, proteolytically cleaved from the CBD tag, and purified using established methods using similar procedures as described in Example 8. The purified RGD-containing cyclic peptides were then tested in an in vitro binding assay with immobilized integrin αᵥβ₃, follow by detection of the bound peptide with an HRP-conjugated anti-FLAG antibody. Many of these peptides showed detectable activity for αᵥβ₃ binding with estimated affinities in the low micromolar range (K_{D}: 10-50 μM).

Based on these results, two cyclopeptides were genetically fused to the Fc domain of human IgG1 to give constructs RGD5R (pCaaF)-Tras-Fc and RGD5R (pCmF)-Tras-Fc (Table 1, Entries 15-16). After purification as described in Example 2, these cyclopeptibodies were used to treat breast cancer cells (MDA-MB-231), which are known to express high levels of integrin αᵥβ₃ on their surface. The cyclopeptibody treated cells were then analyzed by flow cytometry using a procedure similar to that described for the PD-L1-targeting cyclopeptibodies in Example 9. Importantly, these experiments demonstrated the ability and functionality of both cyclopeptibody to label the cells (FIG. 18B), whereas the Fc domain alone and the secondary anti-IgG1-Fc antibody show minimal or significantly reduced staining.

Overall, these results demonstrate the utility of the methods disclosed herein for the successful generation of functional integrin-targeting cyclopeptibodies. As shown here, these compounds can be applied for biomarker detection and imaging in cancer cells. As for the cyclopeptibody molecules described in the previous Examples, several other applications for these compounds can be easily envisioned, including detection and/or staining of the target protein in vitro and in vivo (e.g., upon conjugation of the cyclopeptibody with a fluorescent dye, IR probe, or PET contrast agent), cell-targeted drug delivery (e.g., upon conjugation of the cyclopeptibody with a drug cargo), and use as therapeutic agents.

### 6.11 Example 11: Stability of Cyclopeptibodies in Human Blood Serum

The proteolytic stability of cyclopeptibodies cCLP003-Tras-Fc and L2-m5-Tras-Fc were assessed by incubating these proteins in human blood serum, followed by measurement of the cyclopeptibody activity using an in vitro binding assay with plate-immobilized PD-L1 or Sh as described in Example 7 and 8, respectively. These experiments showed that the half-life of cCLP003-Tras-Fc in human blood serum is longer than 24 hrs. (FIG. 19A), while no loss in activity was observed for L2-m5-Tras-Fc over 24 h (FIG. 19B), indicating a half-life >30-50 hours. These results show that cyclopeptibodies show significant stability in human blood serum, which is desirable for the application of these compounds in in vivo studies and for therapeutic purposes.

### Experimental Details.

Serum stability Assay. The human male serum (Sigma-Aldrich) was clarified by centrifugation at 14 000 rpm for 15 min. 50% serum was prepared in phosphate-buffered saline (pH 7.4), then incubated at 37° C. Cyclopeptiodies were dissolved in 30 μL of 50% serum at a final concentration of 1 μM, incubated at 37° C. Serum samples with diluted cyclopeptibodies were prepared in the same way described above over a time course of 30 h for cCLP003-Tras-Fc-His and 24 h for L2-m5-Tras-Fc-His, followed by incubated at 37° C. Intact cyclopeptibodies in serum were quantified using the binding assay described above. In the assay, immobilized PD-L1 or Shh was incubated with 100 μL of serum samples diluted with PBS (1:10 dilution for cCLP003-Tras-Fc-His, 1:100 dilution for L2-m5-Tras-Fc-His). Intact cyclopeptibody in each sample was detected with HRP-conjugate mouse anti-FLAG polyclonal antibody. The absolute concentration of intact cyclopeptibody in each serum sample was quantified using a calibration curve, which was generated from a binding assay in which immobilized PD-L1 or Shh was incubated with cyclopeptibody in serial dilution (0-100 nM for cCLP003-Tras-Fc-His; 0-10 nM for L2-m5-Tras-Fc-His), followed by detected with HRP-conjugate mouse anti-FLAG polyclonal antibody, absorbance at 450 nm was measured as a function of cyclopeptibody concentration.

### 6.14 Example 12: Application of Cyclopeptibodies as Imaging Agents

Cyclopeptibodies can be applied for detection and imaging of a target protein in cells and other biological systems. To illustrate this aspect of the invention, breast cancer cells MDA-MB-231 cells were treated with PD-L1 targeting cRK10 (pCaaF)-Tras-Fc. MDA-MB-231 cells are known to express high levels of PD-L1 on the cell membrane. After treatment with cRK10 (pCaaF)-Tras-Fc, the cyclopeptibody bound to the surface PD-L1 was labeled with fluorescein-conjugated anti-human Fc antibody (‘anti-Fc-FITC’) and cells were imaged using confocal microscopy. These experiments showed that cRK10 (pCaaF)-Tras-Fc is able to efficiently recognize PD-L1 expressed on the surface of MDA-MB-231 cells, as determined by fluorescent imaging of the cells after treatment with the cyclopeptibody followed by staining with a fluorescein-conjugated anti-Fc antibody (‘anti-Fc-FITC’) (FIG. 17C). No background labeling was observed in control experiments in the absence of cyclopeptibodies. Overall, these experiments demonstrated the functionality of two PD-L1-targeting cyclopeptibodies for specific detection and labeling of PD-L1 expressed on cancer cells, supporting their potential value for biomarker detection and tumor imaging purposes.

### 6.12 Example 13: Cyclopeptibody Molecule with Variable Spacer Sequences

Spacer sequences can be introduced at various positions of the cyclopeptibody molecule to modulate its stability and/or protein binding properties. For example, variable spacer sequences can be introduced between the cyclic peptide and the Fc domain, or fragment thereof, in the cyclopeptibody molecule for the purpose of improving stability of the construct and/or enhance affinity toward the target protein.

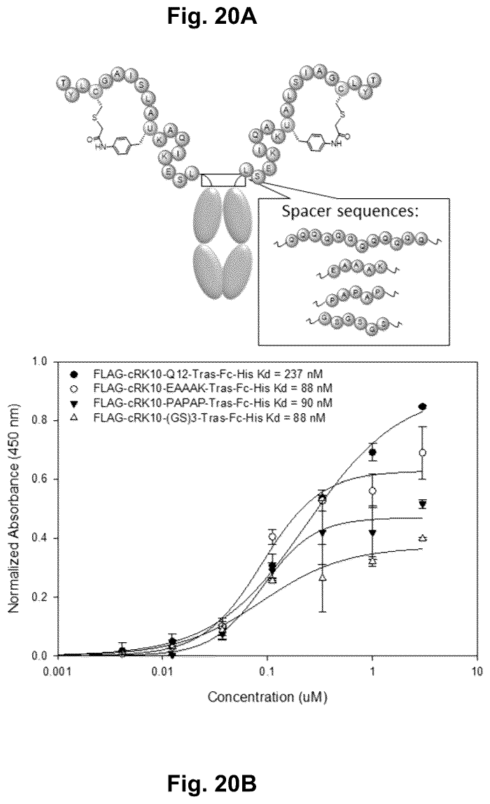

To illustrate this aspect of the invention, a series of PD-L1 targeting cyclopeptibodies were prepared by introducing variable spacer sequences between the cyclic peptide and Fc domain in the PD-L1-targeting cyclopeptibody cRK10-Tras-Fc (Table 1, Entries 31-34; FIG. 20). The spacer sequences included a more flexible (-GSGSGS-), more rigid (-PAPAP-; -EAAAK-) or longer linker (Q12) compared to the exa-glutamine (Q6) spacer sequence in cyclopeptibody cRK10-Tras-Fc. After expression in E. coli and purification via Ni-affinity chromatography, these cyclopeptibodies were determined to bind to PD-L1 with high binding affinity (K_{D}=90-240 nM; FIG. 20). Whereas the cyclopeptibodies with the shorter spacers were found to exhibit similar PD-L1 binding affinity (K_{D}=88-90 nM), the more rigid spacers (-PAPAP-; -EAAAK-) show improved proteolytic stability compared to the more flexible spacer (-GSGSGS-). On the other hand, cyclopeptibody cRK10-Tras-Fc shows a six-fold higher PD-L1 binding affinity (K_{D}=42 vs. 237 nM; FIGS. 15 and 20) compared to cyclopeptibody FLAG-cRK10-Q12-Tras-Fc-His, which contains the same cyclic peptide and Fc domain but separated by a longer polyglutamine spacer (Q12 vs. Q6). This example demonstrates the possibility of utilizing different spacer sequences within cyclopeptibody sequence for modulating its stability and/or protein binding properties.

### 6.13 Example 14: Polyvalent Cyclopeptibodies

Unlike antibodies, cyclopeptibodies can be designed to easily incorporate multiple copies of the same protein-binding cyclic peptide to the same chain of an immunoglobulin Fc domain. Upon spontaneous homodimerization of the immunoglobulin Fc domain, a cyclopeptibody containing multiple copies (i.e., >2) of the same protein-binding cyclic peptide is obtained. This polyvalency can results in enhanced affinity and/or specificity toward the protein targeted by the cyclic peptides, compared to a cyclopeptibody containing a single copy of the cyclic peptide, due to avidity effects.

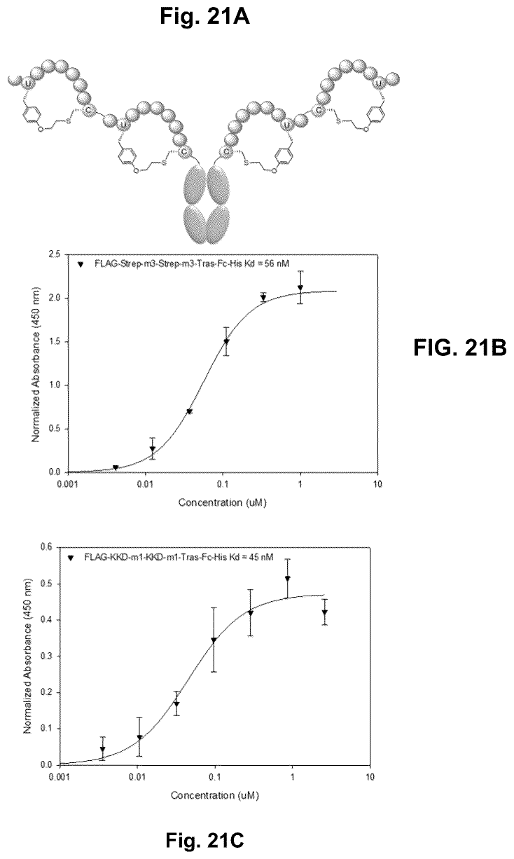

To illustrate this aspect of the invention, a polyvalent streptavidin-targeting cyclopeptibody was prepared by fusing two copies of the O2beY-linked Strep-m3 cyclic peptide to the N-terminus of human IgG₁ Fc containing a C-terminal poly-His tag (Table 1, Entry 35). The resulting cyclopeptibody (called ‘FLAG-Strep-m3-Strep-m3-Tras-Fc-His’) was expressed, purified, and characterized as described in Example 4. The cyclopeptibody FLAG-Strep-m3-Strep-m3-Tras-Fc-His was then characterized for its streptavidin binding affinity using a reported in vitro binding assay (Owens et al, ACS Cent Sci. (2020); 6(3):368-381). These experiments showed that cyclopeptibody FLAG-Strep-m3-Strep-m3-Tras-Fc-His is able to bind streptavidin with high affinity (K_{D}=56 nM) (FIG. 21).

As another example, a polyvalent Keap1-targeting cyclopeptibody was prepared by fusing two copies of the O2beY-linked KKD-m1 cyclic peptide to the N-terminus of human IgG₁ Fc containing a C-terminal poly-His tag (Table 1, Entry 36). The resulting cyclopeptibody (called ‘FLAG-KKD-m1-KKD-m1-Tras-Fc-His’) was expressed, purified, and characterized as described in Example 4. The cyclopeptibody FLAG-KKD-m1-KKD-m1-Tras-Fc-His was then characterized for Keap1 binding affinity using a reported in vitro binding assay (Owens et al, ACS Cent Sci. (2020); 6(3):368-381). These experiments showed that cyclopeptibody FLAG-KKD-m1-KKD-m1-Tras-Fc-His is able to bind Keap1 with very high affinity (K_{D}=45 nM) (FIG. 21).

### 6.14 Example 15: Bispecific Cyclopeptibodies

As described above, bispecific cyclopeptibodies can be prepared by fusing cyclic peptides with different target specificity to the same or different immunoglobulin Fc domain molecule. In some embodiments, bispecific cyclopeptibodies can be prepared by combining two chains of an immunoglobulin Fc domain molecule, each incorporating a cyclic peptide with different specificity toward a target protein of interest. This specific application benefits from the use of engineered chains of an immunoglobulin Fc domain molecule which contain mutations that favor heterodimerization of the Fc domain chains over homodimerization, e.g., through a so-called knob-into-hole strategy (Ma et al., Frontier Immunology (2021); 12:626616).

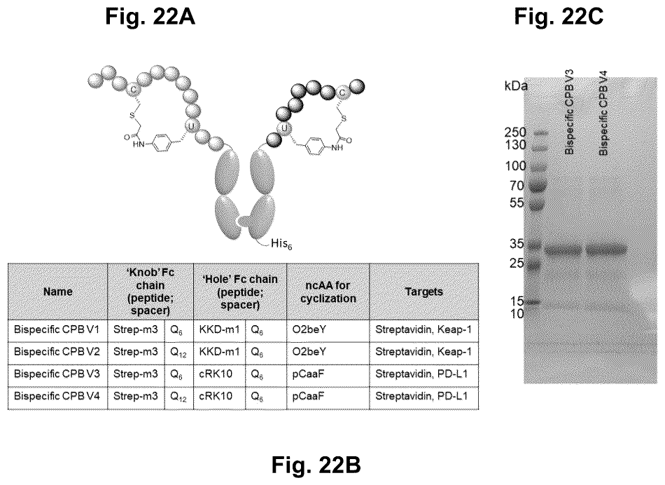

To illustrate this aspect of the invention, a bispecific streptavidin/PD-L1 targeting cyclopeptibody was prepared by fusing a PD-L1-targeting cyclic peptide (cRK10) cyclized via pCaaF to the N-terminus of human IgG₁ Fc domain containing the mutations Y349C/T366S/L368A/Y407V to form a ‘hole’ Fc chain. A second Fc-fusion construct was prepared by fusing a streptavidin-binding cyclic peptide (Strep-m3) cyclized by pCaaF to the N-terminus of human IgG₁ Fc domain containing the mutations S354C/T366W to form a ‘knob’ Fc chain. This construct was also designed to contain a poly-His tag to facilitate purification of the desired bispecific cyclopeptibody (Table 1, Entries 39-40; FIG. 22). The two cyclic peptide-Fc fusion constructs were co-expressed in E. coli using a pET_Duet vector and an amber stop suppression system for genetic incorporation of pCaaF. The bispecific cyclopeptibody was purified using Ni-affinity chromatography. SDS-PAGE and MALDI-TOF MS spectrometry confirmed the purity of the construct and the heterodimeric composition of the isolated construct (FIG. 22). In addition, the cyclopeptibody is able to bind both streptavidin and PD-L1 in the corresponding in vitro binding assays.

As another example, a bispecific streptavidin/Keap1 targeting cyclopeptibody was prepared by fusing a Keap1-targeting cyclic peptide (KKD-m1) cyclized via O2beY to human IgG₁ Fc domain containing the mutations Y349C/T366S/L368A/Y407V to form the ‘hole’ Fc chain. A second Fc-fusion construct was prepared by fusing a streptavidin-binding cyclic peptide (Strep-m3) cyclized by O2beY to his-tagged human IgG₁ Fc domain containing the mutations S354C/T366W to form the ‘knob’ Fc chain (FIG. 22; Table 1, Entries 37-38). The two cyclic peptide-Fc fusion constructs were co-expressed in E. coli using a pET_Duet vector and an amber stop suppression system for genetic incorporation of O2beY. The bispecific cyclopeptibody was purified using Ni-affinity chromatography and its purity and heterodimeric composition was confirmed by SDS-PAGE and MALDI-TOF MS spectrometry, respectively. In addition, the cyclopeptibody is able to bind both streptavidin and Keap1 in the corresponding in vitro binding assays. Altogether, these examples demonstrating the design and isolation of functional bispecific cyclopeptibodies according to the methods described herein.

### REFERENCES

Anderson, J. C., N. Wu, S. W. Santoro, V. Lakshman, D. S. King, and P. G. Schultz. 2004. ‘An expanded genetic code with a functional quadruplet codon’, Proc Natl Acad Sci USA, 101:7566-71.
Bessho, Y., D. R. Hodgson, and H. Suga. 2002. ‘A tRNA aminoacylation system for non-natural amino acids based on a programmable ribozyme’, Nat Biotechnol, 20:723-8.
Dedkova, L. M., N. E. Fahmi, S. Y. Golovine, and S. M. Hecht. 2003. ‘Enhanced D-amino acid incorporation into protein by modified ribosomes’, Journal of the American Chemical Society, 125:6616-17.
Dias, R. L. A., R. Fasan, K. Moehle, A. Renard, D. Obrecht, and J. A. Robinson. 2006. ‘Protein ligand design: From phage display to synthetic protein epitope mimetics in human antibody Fc-binding peptidomimetics’, J. Am. Chem. Soc., 128:2726-32.
Fairlie, D. P., J. D. A. Tyndall, R. C. Reid, A. K. Wong, G. Abbenante, M. J. Scanlon, D. R. March, D. A. Bergman, C. L. L. Chai, and B. A. Burkett. 2000. ‘Conformational selection of inhibitors and substrates by proteolytic enzymes: Implications for drug design and polypeptide processing’, J. Med. Chem., 43:1271-81.
Fekner, T., and M. K. Chan. 2011. ‘The pyrrolysine translational machinery as a genetic-code expansion tool’, Current Opinion in Chemical Biology, 15:387-91.
Frost, J. R., F. Vitali, N. T. Jacob, M. D. Brown, and R. Fasan. 2013. ‘Macrocyclization of Organo-Peptide Hybrids through a Dual Bio-orthogonal Ligation: Insights from Structure-Reactivity Studies’, Chembiochem, 14:147-60.
Hartman, M. C., K. Josephson, C. W. Lin, and J. W. Szostak. 2007. ‘An expanded set of amino acid analogs for the ribosomal translation of unnatural peptides’, PLOS One, 2: e972.
Hartman, M. C., K. Josephson, and J. W. Szostak. 2006. ‘Enzymatic aminoacylation of tRNA with unnatural amino acids’, Proc Natl Acad Sci USA, 103: 4356-61.
Henchey, L. K., J. R. Porter, I. Ghosh, and P. S. Arora. 2010. ‘High Specificity in Protein Recognition by Hydrogen-Bond-Surrogate alpha-Helices: Selective Inhibition of the p53/MDM2 Complex’, Chembiochem, 11:2104-07.
Josephson, K., M. C. Hartman, and J. W. Szostak. 2005. ‘Ribosomal synthesis of unnatural peptides’, J Am Chem Soc, 127:11727-35.
Kourouklis, D., H. Murakami, and H. Suga. 2005. ‘Programmable ribozymes for mischarging tRNA with nonnatural amino acids and their applications to translation’, Methods, 36:239-44.
Lang, K., and J. W. Chin. 2014. ‘Cellular incorporation of unnatural amino acids and bioorthogonal labeling of proteins’, Chem. Rev., 114:4764-806.
Liu, C. C., and P. G. Schultz. 2010. ‘Adding new chemistries to the genetic code’, Annu. Rev. Biochem., 79:413-44.
Murakami, H., A. Ohta, H. Ashigai, and H. Suga. 2006. ‘A highly flexible tRNA acylation method for non-natural polypeptide synthesis’, Nat Methods, 3:357-9.
Neumann, H., A. L. Slusarczyk, and J. W. Chin. 2010. ‘De novo generation of mutually orthogonal aminoacyl-tRNA synthetase/tRNA pairs’, J Am Chem Soc, 132:2142-4.
Neumann, H., K. Wang, L. Davis, M. Garcia-Alai, and J. W. Chin. 2010. ‘Encoding multiple unnatural amino acids via evolution of a quadruplet-decoding ribosome’, Nature, 464:441-4.
Owens, A. E., K. T. Grasso, C. A. Ziegler, and R. Fasan. 2017. ‘Two-Tier Screening Platform for Directed Evolution of Aminoacyl-tRNA Synthetases with Enhanced Stop Codon Suppression Efficiency’, Chembiochem, 18:1109-16.
Rezai, T., J. E. Bock, M. V. Zhou, C. Kalyanaraman, R. S. Lokey, and M. P. Jacobson. 2006. ‘Conformational flexibility, internal hydrogen bonding, and passive membrane permeability: Successful in silico prediction of the relative permeabilities of cyclic peptides’, Journal of the American Chemical Society, 128:14073-80.
Rezai, T., B. Yu, G. L. Millhauser, M. P. Jacobson, and R. S. Lokey. 2006. ‘Testing the conformational hypothesis of passive membrane permeability using synthetic cyclic peptide diastereomers’, Journal of the American Chemical Society, 128:2510-11.
Rodriguez, E. A., H. A. Lester, and D. A. Dougherty. 2006. ‘In vivo incorporation of multiple unnatural amino acids through nonsense and frameshift suppression’, Proc Natl Acad Sci USA, 103:8650-5.
Smith, J. M., F. Vitali, S. A. Archer, and R. Fasan. 2011. ‘Modular Assembly of Macrocyclic Organo-Peptide Hybrids Using Synthetic and Genetically Encoded Precursors’, Angew Chem Int Ed, 50:5075-80.
Tang, Y. Q., J. Yuan, G. Osapay, K. Osapay, D. Tran, C. J. Miller, A. J. Ouellette, and M. E. Selsted. 1999. ‘A cyclic antimicrobial peptide produced in primate leukocytes by the ligation of two truncated alpha-defensins’, Science, 286:498-502.
Theunissen, J. W., and F. J. de Sauvage. 2009. ‘Paracrine Hedgehog signaling in cancer’, Cancer Res., 69:6007-10.
Tian, H., C. A. Callahan, K. J. DuPree, W. C. Darbonne, C. P. Ahn, S. J. Scales, and F. J. de Sauvage. 2009. ‘Hedgehog signaling is restricted to the stromal compartment during pancreatic carcinogenesis’, Proc. Natl. Acad. Sci. USA, 106:4254-9.
Walensky, L. D., A. L. Kung, I. Escher, T. J. Malia, S. Barbuto, R. D. Wright, G. Wagner, G. L. Verdine, and S. J. Korsmeyer. 2004. ‘Activation of apoptosis in vivo by a hydrocarbon-stapled BH3 helix’, Science, 305:1466-70.
Wang, D., W. Liao, and P. S. Arora. 2005. ‘Enhanced metabolic stability and protein-binding properties of artificial alpha helices derived from a hydrogen-bond surrogate: application to Bcl-xL’, Angew Chem Int Ed Engl, 44:6525-9.
Wang, L., J. Xie, and P. G. Schultz. 2006. ‘Expanding the genetic code’, Annu Rev Biophys Biomol Struct, 35:225-49.
Wu, X., and P. G. Schultz. 2009. ‘Synthesis at the interface of chemistry and biology’, J Am Chem Soc, 131:12497-515.
Yauch, R. L., S. E. Gould, S. J. Scales, T. Tang, H. Tian, C. P. Ahn, D. Marshall, L. Fu, T. Januario, D. Kallop, M. Nannini-Pepe, K. Kotkow, J. C. Marsters, L. L. Rubin, and F. J. de Sauvage. 2008. ‘A paracrine requirement for hedgehog signalling in cancer’, Nature, 455:406-U61.
Young, D. D., T. S. Young, M. Jahnz, I. Ahmad, G. Spraggon, and P. G. Schultz. 2011. ‘An evolved aminoacyl-tRNA synthetase with atypical polysubstrate specificity’, Biochemistry, 50:1894-900.

TABLE 1
Sequences and activity of cyclopeptibodies described in the application.

| | | SEQ | | |
|---|---|---|---|---|
| Entry | Sequence | ID NO | Z | Activity |
| 1 | FLAG-(GS)₂-SCWWHPQGDZ-(Q)₆-(Fc)¹-H₆ | 201 | O2beY | Streptavidin binding |
| 2 | FLAG-(GS)₂-SCWWHPQGDZ-(Q)₆-(Fc)¹ | 202 | O2beY | Streptavidin binding |
| 3 | FLAG-(GS)₂-ZDSETGEC-(Q)₆-(Fc)¹-H₆ | 203 | O2beY | Keap1 |
| | | | | binding/inhibition |
| 4 | FLAG-(GS)₂-ZDSETGEC-(Q)₆-(Fc)¹ | 204 | O2beY | Keap1 |
| | | | | binding/inhibition |
| 5 | FLAG-(GS)₂-TLSWZEAMDMCTSS-(Q)₆-(Fc)¹-H₆ | 205 | O2beY | Hedgehog |
| | | | | binding/inhibition |
| 6 | FLAG-(GS)₂-TLSWZEAMDMCTSS-(Q)₆-(Fc)¹ | 206 | O2beY | Hedgehog |
| | | | | binding/inhibition |
| 7 | FLAG-(GS)₂-TLSWZEAMDMCTSS-(GS)₃-(Fc)¹ | 207 | O2beY | Hedgehog |
| | | | | binding/inhibition |
| 8 | FLAG-(GS)₂-TLSWZEAMDMCTSS-(Q)₆-IEGR-(Fc)² | 208 | O2beY | Hedgehog |
| | | | | binding/inhibition |
| 9 | FLAG-(GS)₂-TLSWZEAMDMCTSS-(Q)₆-IEGR-(Fc)² | 209 | pCaaF | Hedgehog |
| | | | | binding/inhibition |
| 10 | FLAG-(GS)₂-TLSWZEAMDMCTSS-(GS)₃-IEGR-(Fc)² | 210 | O2beY | Hedgehog |
| | | | | binding/inhibition |
| 11 | FLAG-(GS)₂-ZVYNRMKCG-(Q)₆-IEGR-(Fc)¹ | 211 | O2beY | Hedgehog |
| | | | | binding/inhibition |
| 12 | (Fc)¹-(Q)₆-GTLSWZEAMDMCTSS | 212 | O2beY | Hedgehog |
| | | | | binding/inhibition |
| 13 | FLAG-(GS)₂-WHFSZNWRWLPC-(Q)₆-(Fc)¹-H₆ | 213 | pCaaF | PD-L1 |
| | | | | binding/inhibition |
| 14 | FLAG-(GS)₂-TYLCGAISLAZKAQIKESL-(Q)₆-(Fc)¹-H₆ | 214 | pCaaF | PD-L1 |
| | | | | binding/inhibition |
| 15 | FLAG-(GS)₂-CRDRGDYZ-(Q)₆-IEGR-(Fc)¹-H₆ | 215 | pCaaF | Integrin receptor |
| | | | | binding |
| 16 | FLAG-(GS)₂-CRDRGDYZ-(Q)₆-IEGR-(Fc)¹-H₆ | 216 | pCmF | Integrin receptor |
| | | | | binding |
| 17 | FLAG-(GS)₂-ZLGRGDYC-(Q)₆-IEGR-(Fc)¹ | 217 | O2beY | Integrin receptor |
| | | | | binding |
| 18 | FLAG-(GS)₂-CLGRGDYZ-(Q)₆-IEGR-(Fc)¹ | 218 | O2beY | Integrin receptor |
| | | | | binding |
| 19 | FLAG-(GS)₂-ZHVRGDNC-(Q)₆-IEGR-(Fc)¹ | 219 | O2beY | Integrin receptor |
| | | | | binding |
| 20 | FLAG-(GS)₂-CHVRGDNZ-(Q)₆-IEGR-(Fc)¹ | 220 | O2beY | Integrin receptor |
| | | | | binding |
| 21 | FLAG-(GS)₂-ZHRGDHLC-(Q)₆-IEGR-(Fc)¹ | 221 | O2beY | Integrin receptor |
| | | | | binding |
| 22 | FLAG-(GS)₂-CHRGDHLZ-(Q)₆-IEGR-(Fc)¹ | 222 | O2beY | Integrin receptor |
| | | | | binding |
| 23 | FLAG-(GS)₂-ZLGRGDNC-(Q)₆-IEGR-(Fc⁾¹ | 223 | O2beY | Integrin receptor |
| | | | | binding |
| 24 | FLAG-(GS)₂-CLGRGDNZ-(Q)₆-IEGR-(Fc)¹ | 224 | O2beY | Integrin receptor |
| | | | | binding |
| 25 | FLAG-(GS)₂-ZRDRGDYC-(Q)₆-IEGR-(Fc)¹ | 225 | O2beY | Integrin receptor |
| | | | | binding |
| 26 | FLAG-(GS)₂-CRDRGDYZ-(Q)₆-IEGR-(Fc)¹ | 226 | O2beY | Integrin receptor |
| | | | | binding |
| 27 | FLAG-(GS)₂-ZGRGDGC-(Q)₆-IEGR-(Fc)¹ | 227 | 02beY | Integrin receptor |
| | | | | binding |
| 28 | FLAG-(GS)₂-CGRGDGZ-(Q)₆-IEGR-(Fc)¹ | 228 | O2beY | Integrin receptor |
| | | | | binding |
| 29 | M-TYLCGAISLAZKAQIKESL-(Q)₆-(Fc)¹-H₆ | 229 | pCaaF | PD-L1 |
| | | | | binding/inhibition |
| 30 | HA-(GS)₂-TYLCGAISLAZKAQIKESL-(Q)₆-(Fc)¹-H₆ | 230 | pCaaF | PD-L1 |
| | | | | binding/inhibition |
| 31 | FLAG-(GS)₂-TYLCGAISLAZKAQIKESL-(GS)₃-(Fc)¹- | 231 | pCaaF | PD-L1 |
| | H₆ | | | binding/inhibition |
| 32 | FLAG-(GS)₂-TYLCGAISLAZKAQIKESL-PAPAP-(Fc)¹- | 232 | pCaaF | PD-L1 |
| | H₆ | | | binding/inhibition |
| 33 | FLAG-(GS)₂-TYLCGAISLAZKAQIKESL-EAAAK- | 233 | pCaaF | PD-L1 |
| | (Fc)¹-H₆ | | | binding/inhibition |
| 34 | FLAG-(GS)₂-TYLCGAISLAZKAQIKESL-(Q)₁₂-(Fc)¹- | 234 | pCaaF | PD-L1 |
| | H₆ | | | binding/inhibition |
| 35 | FLAG-(GS)₂-SCWWHPQGDZ-(GS)₂-ENLYFQ-(GS)₂- | 235 | O2beY | Streptavidin binding |
| | SCWWHPQGDZIEGR-(GS)₂-(Fc)¹-H₆ | | | (polyvalent) |
| 36 | FLAG-(GS)₂-ZDSETGEC -(GS)₂-ENLYFQ-(GS)₂- | 236 | O2beY | Keap1 |
| | ZDSETGEC-IEGR-(GS)₂-(Fc)¹-H₆ | | | binding/inhibition |
| | | | | (polyvalent) |
| 37 | FLAG-(GS)₂-ZDSETGEC-(Q)₆-(Fc)² | 237 | O2beY | Keap1 |
| | FLAG-(GS)₂-SCWWHPQGDZ-(Q)₆-(Fc)³-H₆ | | | binding/inhibition; |
| | | | | Streptavidin binding |
| | | | | (bispecific) |
| 38 | FLAG-(GS)₂-ZDSETGEC-(Q)₃-IGER-(Q)₃-(Fc)² | 238 | O2beY | Keap1 |
| | FLAG-(GS)₂-SCWWHPQGDZ-(Q)₆-IGER-(Q)₆-(Fc)³-H₆ | | | binding/inhibition; |
| | | | | Streptavidin binding |
| | | | | (bispecific) |
| 39 | FLAG-(GS)₂-TYLCGAISLAZKAQIKESL-(Q)₆-(Fc)² | 239 | pCaaF | PD-L1 |
| | FLAG-(GS)₂-SCWWHPQGDZ-(Q)₆-(Fc)³-H₆ | | | binding/inhibition; |
| | | | | Streptavidin binding |
| | | | | (bispecific) |
| 40 | FLAG-(GS)₂-TYLCGAISLAZKAQIKESL-(Q)₆-(Fc)² | 240 | pCaaF | PD-L1 |
| | FLAG-(GS)₂-SCWWHPQGDZ-(Q)₆-IGER-(Q)₆-(Fc)³-H₆ | | | binding/inhibition; |
| | | | | Streptavidin binding |
| | | | | (bispecific) |

Where “(Fc)¹” is an Fc domain and “Z” is a non-canonical amino acid, as defined previously herein. The Cys residue involved in cyclization with the Z residue is highlighted in bold and underlined. FLAG corresponds to a FLAG tag (SEQ ID NO: 124). The cysteine residue involved in the thioether linkage with the Z amino acid is underlined. HA corresponds to a HA tag (YPYDVPDYA). “(Fc)²” and “(Fc)³” correspond to a human IgG₁ Fc containing Y349C/T366S/L368A/Y407V mutations to form the ‘hole’ and a human IgG1 Fc containing S354C/T366W mutations to form the ‘knob’, respectively.

TABLE 2
Sequences of RGD-containing cyclic peptides. CBD is chitin binding domain,
ncAA is the non-canonical amino acid, and FLAG corresponds to a FLAG tag.
The cysteine residue involved in the thioether linkage with the Y* amino
acid is underlined.

| Entry | Sequence | ncAA | Sequence |
|---|---|---|---|
| 1 | FLAG-(GS)₂-Y*HVRGDNC-CBD-His₆ | O2beY | SEQ ID NO: 241 |
| 2 | FLAG-(GS)₂-Y*HVRGDNC-CBD-His₆ | pCaaF | SEQ ID NO: 242 |
| 3 | FLAG-(GS)₂-Y*HVRGDNC-CBD-His₆ | pCmF | SEQ ID NO: 243 |
| 4 | FLAG-(GS)₂-CHVRGDNY*-CBD-His₆ | O2beY | SEQ ID NO: 244 |
| 5 | FLAG-(GS)₂-CHVRGDNY*-CBD-His₆ | pCaaF | SEQ ID NO: 245 |
| 6 | FLAG-(GS)₂-CHVRGDNY*-CBD-His₆ | pCmF | SEQ ID NO: 246 |
| 7 | FLAG-(GS)₂-Y*HRGDHLC-CBD-His₆ | O2beY | SEQ ID NO: 247 |
| 8 | FLAG-(GS)₂-Y*HRGDHLC-CBD-His₆ | pCaaF | SEQ ID NO: 248 |
| 9 | FLAG-(GS)₂-Y*HRGDHLC-CBD-His₆ | pCmF | SEQ ID NO: 249 |
| 10 | FLAG-(GS)₂-CHRGDHLY*-CBD-His₆ | O2beY | SEQ ID NO: 250 |
| 11 | FLAG-(GS)₂-CHRGDHLY*-CBD-His₆ | pCaaF | SEQ ID NO: 251 |
| 12 | FLAG-(GS)₂-CHRGDHLY*-CBD-His₆ | pCmF | SEQ ID NO: 252 |
| 13 | FLAG-(GS)₂-Y*RDRGDYC-CBD-His₆ | O2beY | SEQ ID NO: 253 |
| 14 | FLAG-(GS)₂-Y*RDRGDYC-CBD-His₆ | pCaaF | SEQ ID NO: 254 |
| 15 | FLAG-(GS)₂-Y*RDRGDYC-CBD-His₆ | pCmF | SEQ ID NO: 255 |
| 16 | FLAG-(GS)₂-CRDRGDYY*-CBD-His₆ | O2beY | SEQ ID NO: 256 |
| 17 | FLAG-(GS)₂-CRDRGDYY*-CBD-His₆ | pCaaF | SEQ ID NO: 257 |
| 18 | FLAG-(GS)₂-CRDRGDYY*-CBD-His6 | pCmF | SEQ ID NO: 258 |

It will be appreciated that variants of the above-disclosed and other features and functions, or alternatives thereof, may be combined into many other different systems or applications. Various presently unforeseen or unanticipated alternatives, modifications, variations, or improvements therein may be subsequently made by those skilled in the art which are also intended to be encompassed by the following claims.

While embodiments of the present disclosure have been particularly shown and described with reference to certain examples and features, it will be understood by one skilled in the art that various changes in detail may be effected therein without departing from the spirit and scope of the present disclosure as defined by claims that can be supported by the written description and drawings. Further, where exemplary embodiments are described with reference to a certain number of elements it will be understood that the exemplary embodiments can be practiced utilizing either less than or more than the certain number of elements.

All references cited herein are incorporated herein by reference in their entirety and for all purposes to the same extent as if each individual publication, patent or patent application was specifically and individually indicated to be incorporated by reference in its entirety for all purposes.

The citation of any publication is for its disclosure prior to the filing date and should not be construed as an admission that the present invention is not entitled to antedate such publication by virtue of prior invention.

1. An artificial polypeptide of structure:
(AA)ₘ-Z-(AA)ₙ-Cys-(AA)ₚ (I)
or
(AA)ₘ-Cys-(AA)ₙ-Z-(AA)ₚ (II)
or
(AA)ₘ-Cys-(AA)ₙ-Z2-(AA)ₒ-Cys-(AA)ₚ (V)
wherein:
i. (AA)ₘ is an N-terminal amino acid or peptide sequence,
ii. Z is a non-canonical amino acid carrying a side-chain functional group FG₁, FG₁ being a functional group selected from the group consisting of —(CH₂)ₙX, where X is F, Cl, Br, or I and n is an integer number from 1 to 10; —C(O)CH₂X, where X is F, Cl, Br, or I; —CH(R′)X, where X is F, Cl, Br, or I; —C(O)CH(R′)X, where X is F, Cl, Br, or I; —OCH₂CH₂X, where X is F, Cl, Br, or I; —C(O)CH═C═C(R′)(R″); —SO₂C(R′)═C(R′)(R″); —C(O)C(R′)═C(R′)(R″); —C(R′)═C(R′)C(O)OR′; —C(R′)═C(R′)C(O)N(R′)(R″); —C(R′)═C(R′)—CN; —C(R′)═C(R′)—NO₂; —C≡C—C(O)OR′; —C≡C—C(O)N(R′)(R″); unsubstituted or substituted oxirane; unsubstituted or substituted aziridine; 1,2-oxathiolane 2,2-dioxide; 4-fluoro-1,2-oxathiolane 2,2-dioxide; and 4,4-difluoro-1,2-oxathiolane 2,2-dioxide, where each R′ and R″ is independently H, an aliphatic, a substituted aliphatic, an aryl, or a substituted aryl group,
iii. Z2 is a non-canonical amino acid carrying two side-chain functional groups FG₁ and FG₂, wherein each of FG₁ and FG₂ is a functional group independently selected from the group consisting of —(CH₂)ₙX, where X is F, Cl, Br, or I and n is an integer number from 1 to 10; —C(O)CH₂X, where X is F, Cl, Br, or I; —CH(R′)X, where X is F, Cl, Br, or I; —C(O)CH(R′)X, where X is F, Cl, Br, or I; —OCH₂CH₂X, where X is F, Cl, Br, or I; —C(O)CH═C═C(R′)(R″); —SO₂C(R′)═C(R′)(R″); —C(O)C(R′)═C(R′)(R″); —C(R′)═C(R′)C(O)OR′; —C(R′)═C(R′)C(O)N(R′)(R″); —C(R′)═C(R′)—CN; —C(R′)═C(R′)—NO₂; —C≡C—C(O)OR′; —C≡C—C(O)N(R′)(R″); unsubstituted or substituted oxirane; unsubstituted or substituted aziridine; 1,2-oxathiolane 2,2-dioxide; 4-fluoro-1,2-oxathiolane 2,2-dioxide; and 4,4-difluoro-1,2-oxathiolane 2,2-dioxide, where each R′ and R″ is independently H, an aliphatic, a substituted aliphatic, an aryl, or a substituted aryl group,
iv. (AA)ₙ is a target peptide sequence,
v. (AA)ₒ is a second target peptide sequence,
vi. (AA)ₚ is a C-terminal amino acid or peptide sequence, and
vii. at least one of (AA)ₚ and (AA)ₘ comprises the Fc region of an immunoglobulin molecule or fragment thereof;
wherein the functional group FG₁, and whenever present, FG₂, react with the side-chain sulfhydryl group (—SH) of the cysteine (Cys) residue(s), thereby producing a thioether-linked cyclic peptide fused to the immunoglobulin Fc region or fragment thereof.

2. The polypeptide of claim 1 wherein Z is an amino acid of structure: 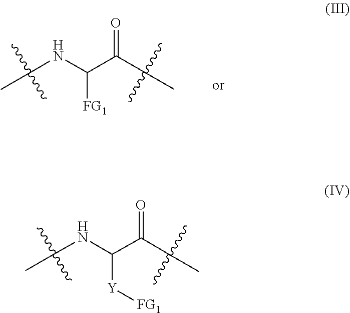
wherein FG₁ is a functional group selected from the group consisting of —(CH₂)ₙX, where X is F, Cl, Br, or and n is an integer number from 1 to 10; —C(O)CH₂X, where X is F, Cl, Br, or I; —CH(R′)X, where X is F, Cl, Br, or I; —C(O)CH(R′)X, where X is F, Cl, Br, or I; —OCH₂CH₂X, where X is F, Cl, Br, or I; —C(O)CH═C═C(R′)(R″); —SO₂C(R′)═C(R′)(R″); —C(O)C(R′)═C(R′)(R″); —C(R′)═C(R′)C(O)OR′; —C(R′)═C(R′)C(O)N(R′)(R″); —C(R′)═C(R′)—CN; —C(R′)═C(R′)—NO₂; —C≡C—C(O)OR′; —C≡C—C(O)N(R′)(R″); unsubstituted or substituted oxirane, unsubstituted or substituted aziridine; 1,2-oxathiolane 2,2-dioxide; 4-fluoro-1,2-oxathiolane 2,2-dioxide; and 4,4-difluoro-1,2-oxathiolane 2,2-dioxide; where each R′ and R″ is independently H, an aliphatic, a substituted aliphatic, an aryl, or a substituted aryl group; and
wherein Y is a linker group selected from the group consisting of aliphatic, aryl, substituted aliphatic, substituted aryl, heteroatom-containing aliphatic, heteroatom-containing aryl, substituted heteroatom-containing aliphatic, substituted heteroatom-containing aryl, alkoxy, and aryloxy groups.

3. The polypeptide of claim 2 wherein Z is an amino acid of structure (IV) and Y is a linker group selected from the group consisting of C₁-C₂₄ alkyl, C₁-C₂₄ substituted alkyl, C₁-C₂₄ substituted heteroatom-containing alkyl, C₁-C₂₄ substituted heteroatom-containing alkyl, C₂-C₂₄ alkenyl, C₂-C₂₄ substituted alkenyl, C₂-C₂₄ substituted heteroatom-containing alkenyl, C₂-C₂₄ substituted heteroatom-containing alkenyl, C₅-C₂₄ aryl, C₅-C₂₄ substituted aryl, C₅-C₂₄ substituted heteroatom-containing aryl, C₅-C₂₄ substituted heteroatom-containing aryl, C₁-C₂₄ alkoxy, and C₅-C₂₄ aryloxy groups.

4. The polypeptide of claim 3 wherein Y is a linker group selected from the group consisting of —CH₂—C₆H₄—, —CH₂—C₆H₄—O—, —CH₂—C₆H₄—NH—, —(CH₂)₄—, —(CH₂)₄NH—, —(CH₂)₄NHC(O)—, and —(CH₂)₄NHC(O)O—.

5. The polypeptide of claim 1 wherein the amino acid Z is selected from the group consisting of 4-(chloromethyl)-phenylalanine, 3-(chloromethyl)-phenylalanine, 4-(2-bromoethoxy)-phenylalanine, 3-(2-bromoethoxy)-phenylalanine, 4-(2-chloroethoxy)-phenylalanine, 4-(4-bromobutoxy)-phenylalanine, 4-(4-chlorobutoxy)-phenylalanine, 3-(4-bromobutoxy)-phenylalanine, 3-(4-bromobutoxy)-phenylalanine, 3-(2-chloroethoxy)-phenylalanine, 4-(1-bromoethyl)-phenylalanine, 3-(1-bromoethyl)-phenylalanine, 4-(aziridin-1-yl)-phenylalanine, 3-(aziridin-1-yl)-phenylalanine, 4-acrylamido-phenylalanine, 3-acrylamido-phenylalanine, 4-(2-fluoro-acetamido)-phenylalanine, 3-(2-fluoro-acetamido)-phenylalanine, 4-(2-chloro-acetamido)-phenylalanine, 3-(2-chloro-acetamido)-phenylalanine, 4-(2-bromo-acetamido)-phenylalanine, 3-(2-bromo-acetamido)-phenylalanine, 4-(acrylamido)-phenylalanine, 3-(acrylamido)-phenylalanine, 4-(vinylsulfonamido)-phenylalanine, 3-(vinylsulfonamido)-phenylalanine, 3-(2-fluoro-acetyl)-phenylalanine, 4-(2-fluoro-acetyl)-phenylalanine, N^{ε}-((2-bromoethoxy)carbonyl)-lysine, N^{ε}-((2-chloroethoxy)carbonyl)-lysine, N^{ε}-(buta-2,3-dienoyl)-lysine, N^{ε}-acryl-lysine, N^{ε}-crotonyl-lysine, N^{ε}-(2-fluoro-acetyl)-lysine, N^{ε}-(2-chloro-acetyl)-lysine, N^{ε}-(2-bromoacetyl)-lysine, and N^{ε}-vinylsulfonyl-lysine.

6. The polypeptide of claim 1 wherein Z2 is an amino acid of structure: 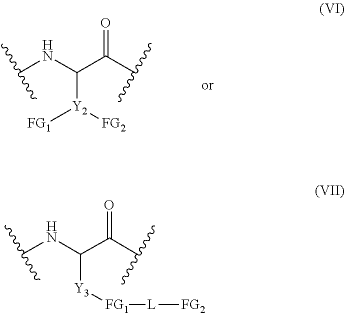
wherein each of FG₁ and FG₂ is a functional group independently selected from the group consisting of —(CH₂)ₙX, where X is F, Cl, Br, or I and n is an integer number from 1 to 10; —C(O)CH₂X, where X is F, Cl, Br, or I; —CH(R′)X, where X is F, Cl, Br, or I; —C(O)CH(R′)X, where X is F, Cl, Br, or I; —OCH₂CH₂X, where X is F, Cl, Br, or I; —C(O)CH═C═C(R′)(R″); —SO₂C(R′)═C(R′)(R″); —C(R′)═C(R′)C(O)OR′; —C(R′)═C(R′)C(O)N(R′)(R″); —C(R′)═C(R′)—CN; —C(R′)═C(R′)—NO₂, —C≡C—C(O)OR′; —C≡C—C(O)N(R′)(R″); unsubstituted or substituted oxirane; unsubstituted or substituted aziridine; 1,2-oxathiolane 2,2-dioxide; 4-fluoro-1,2-oxathiolane 2,2-dioxide; and 4,4-difluoro-1,2-oxathiolane 2,2-dioxide, where each R′ and R″ is independently H, an aliphatic, a substituted aliphatic, an aryl, or a substituted aryl group; and
wherein Y₂, Y₃, and L are linker groups independently selected from the group consisting of aliphatic, aryl, substituted aliphatic, substituted aryl, heteroatom-containing aliphatic, heteroatom-containing aryl, substituted heteroatom-containing aliphatic, substituted heteroatom-containing aryl, alkoxy, and aryloxy groups.

7. The polypeptide of claim 6 wherein Z2 is an amino acid of structure (VI) and Y₂ is a linker group selected from the group consisting of C₁-C₂₄ alkyl, C₁-C₂₄ substituted alkyl, C₁-C₂₄ substituted heteroatom-containing alkyl, C₁-C₂₄ substituted heteroatom-containing alkyl, C₂-C₂₄ alkenyl, C₂-C₂₄ substituted alkenyl, C₂-C₂₄ substituted heteroatom-containing alkenyl, C₂-C₂₄ substituted heteroatom-containing alkenyl, C₅-C₂₄ aryl, C₅-C₂₄ substituted aryl, C₅-C₂₄ substituted heteroatom-containing aryl, C₅-C₂₄ substituted heteroatom-containing aryl, C₁-C₂₄ alkoxy, and C₅-C₂₄ aryloxy groups.

8. The polypeptide of claim 7 wherein Y₂ is a linker group selected from the group consisting of —CH₂—C₆H₄—, —CH₂—C₆H₄—O—, —CH₂—C₆H₄—NH—, —CH₂—C₆H₄—OCH₂, —(CH₂)₄NH—, —(CH₂)₄NHC(O)—, —(CH₂)₄NHC(O)O—, —(CH₂)₄NHC(O)OCH₂—,

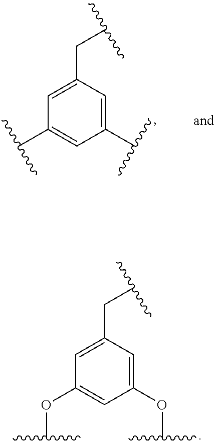 [SMILES: *Cc1cc(*)cc(*)c1 ; *Cc1cc(O*)cc(O*)c1]

9. The polypeptide of claim 1 wherein the amino acid Z2 is selected from the group consisting of 3,5-bis(chloromethyl)-phenylalanine, 3,5-bis(2-bromoethoxy)-phenylalanine, 3,5-bis(2-chloroethoxy)-phenylalanine, 3,5-bis(4-bromobutoxy)-phenylalanine, 3,5-bis(4-chlorobutoxy)-phenylalanine, 3,5-bis(1-bromoethyl)-phenylalanine, 3,5-bis(4-acrylamido)-phenylalanine, 3,5-bis(2-chloro-acetamido)-phenylalanine, 3,5-bis(2-bromo-acetamido)-phenylalanine, 3,5-bis(vinylsulfonamido)-phenylalanine, 3,5-bis(aziridin-1-yl)-phenylalanine, 3,5-bis-acrylamido-phenylalanine, 3,5-bis(2-fluoro-acetamido)-phenylalanine, 3,5-bis(2-fluoro-acetyl)-phenylalanine, 4-((1,3-dibromopropan-2-yl)oxy)-phenylalanine, 4-((1,3-dichloropropan-2-yl)oxy)-phenylalanine, N^{ε}-(((1,3-dibromopropan-2-yl)oxy)carbonyl)-lysine, N^{ε}-(((1,3-dichloropropan-2-yl)oxy)carbonyl)-lysine, 4-(2,3-dibromopropoxy)-phenylalanine, 3-(2,3-dibromopropoxy)-phenylalanine, 4-(2,3-dichloropropoxy)-phenylalanine, 3-(2,3-dichloropropoxy)-phenylalanine, N^{ε}-((2,3-dibromopropoxy)carbonyl)-lysine, N^{ε}-((2,3-dichloropropoxy)carbonyl)-lysine, N^{ε}-bis-(acryl)-lysine, N^{ε}-bis-(crotonyl)-lysine, N^{ε}-bis-(2-fluoro-acetyl)-lysine, N^{ε}-bis-(2-chloro-acetyl)-lysine, N^{ε}-bis-(2-bromoacetyl)-lysine, N^{ε}-bis-(vinylsulfonyl)-lysine, 4-(2,2-dichloro-acetamido)-phenylalanine, 4-(2,2-difluoro-acetamido)-phenylalanine, 3-(2,2-dichloro-acetamido)-phenylalanine, 3-(2,2-difluoro-acetamido)-phenylalanine, 4-(2,2-dichloroacetyl)-phenylalanine, 4-(2,2-difluoroacetyl)-phenylalanine, 3-(2,2-dichloroacetyl)-phenylalanine, and 3-(2,2-difluoroacetyl)-phenylalanine.

10. The polypeptide of claim 1, wherein the codon encoding for Z or Z2 is an amber stop codon TAG, an ochre stop codon TAA, an opal stop codon TGA, or a four base codon.

11. The polypeptide of claim 1, wherein the expression system used to produce it comprises an aminoacyl-tRNA synthetase polypeptide or an engineered variant thereof that is at least 80% identical to any of the polypeptides of SEQ ID NO: 31 to 76; and a transfer RNA molecule encoded by a polynucleotide that is at least 80% identical to any of the polynucleotides of SEQ ID NO: 101 to 120.

12. The polypeptide of claim 11, wherein the expression system used to produce it comprises an aminoacyl-tRNA synthetase polypeptide or an engineered variant thereof that is at least 90% identical to any of the polypeptides of SEQ ID NO: 31 to 76.

13. The polypeptide of claim 11, wherein the expression system used to produce it comprises a transfer RNA molecule encoded by a polynucleotide that is at least 90% identical to any of the polynucleotides of SEQ ID NO: 101 to 120.

14. The polypeptide of claim 11, wherein the expression system used to produce it comprises an aminoacyl-tRNA synthetase polypeptide or an engineered variant thereof that is at least 95% identical to any of the polypeptides of SEQ ID NO: 31 to 76.

15. The polypeptide of claim 11, wherein the expression system used to produce it comprises a transfer RNA molecule encoded by a polynucleotide that is at least 95% identical to any of the polynucleotides of SEQ ID NO: 101 to 120.

16. (canceled)

17. The polypeptide of claim 1, wherein the Fc domain is derived from a human or mouse IgG, IgM, IgA, or IgE.

18. (canceled)

19. The polypeptide of claim 17, wherein the Fc domain comprises a polypeptide that is at least 90% identical to SEQ ID NO: 1, 2, 3, 4, 5, 6, 7, 8, or 9.

20. (canceled)

21. The polypeptide of claim 19, wherein the Fc domain is an engineered variant of human IgG1 of SEQ ID NO: 1 which comprises an amino acid substitution at a position selected from the group consisting of position: X351, X366, X368, X382, X395, X409, X428, and X434 in the corresponding full-length IgG1.

22. (canceled)

23. (canceled)

24. The polypeptide of claim 1 [23], wherein the cyclic peptide is able to bind to Programmed Death-Ligand 1, a Hedgehog protein, or an integrin protein.

25. (canceled)

26. The polypeptide of claim 24, wherein the polypeptide is at least 90% identical to SEQ ID NO: 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 239 or 240.

27. (canceled)

28. The polypeptide of claim 1 [23], wherein the cyclic peptide is able to bind to Kelch-like ECH-associated protein 1 (Keap1) or streptavidin.

29. (canceled)

30. The polypeptide of claim 28, wherein the polypeptide is at least 90% identical to SEQ ID NO: 201, 202, 203, 204, 235, 236, 237, 238, 239 or 240.

31. (canceled)

32. The polypeptide of claim 1 wherein the cyclic peptide is SEQ ID NO: 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, or 240.

33. A pharmaceutical composition for the prevention or treatment of cancer comprising the cyclic peptide according to claim 1 as an active ingredient.

34. A tumor imaging and/or detection agent comprising the cyclic peptide according to claim 1 as an active ingredient.

35. (canceled)

36. (canceled)

37. (canceled)

38. (canceled)

39. The polypeptide of claim 1, wherein multiple copies of the same cyclic peptide are fused to the Fc region of an immunoglobulin molecule or fragment thereof, resulting in a polyvalent cyclopeptibody.

40. The polypeptide of claim 1, wherein two or more cyclic peptides with different binding specificity are fused to the Fc region of an immunoglobulin molecule or fragment thereof, resulting in a polyspecific cyclopeptibody.

41. The polypeptide of claim 40, wherein the cyclopeptibody molecule is a heterodimeric bispecific cyclopeptibody.

42. The polypeptide of claim 39 wherein the cyclopeptibody is able to bind to Kelch-like ECH-associated protein 1 (Keap1) and streptavidin.

43. The polypeptide of claim 39 wherein the cyclopeptibody is able to bind to streptavidin and PD-L1.

44. (canceled)

45. The polypeptide of claim 42 wherein the polypeptide is at least 90% identical to SEQ ID NO: 237 or 238.

46. (canceled)

47. (canceled)

48. The polypeptide of claim 43 wherein the polypeptide is at least 90% identical to SEQ ID NO: 239 or 240.

49. (canceled)
